SEQUENCE LISTING (93 sequences)

SEQ ID NO: 1 - protein, 31 aa, Artificial Sequence
  A synthetic inclusion body fusion partner.
AEEEEILLEVSLVFKVKEFAPDAPLFTGPAY

SEQ ID NO: 2 - protein, 14 aa, Artificial Sequence
  A synthetic T7 tag sequence.
MASMTGGQQMGRGS

SEQ ID NO: 3 - DNA, 93 bp, Artificial Sequence
  A synthetic nucleotide sequence of an inclusion body fusion partner.
gctgaagaagaagaaattttattagaagtttctttagtttttaaagttaaagaatttgctcctgatgctcctttatttactggtcctgcttat

SEQ ID NO: 4 - protein, 30 aa, Unknown
  GLP-1(7-36).
HAEGTFTSDVSSYLEGQAAKEFIAWLVKGR

SEQ ID NO: 5 - protein, 31 aa, Unknown
  GLP-1(7-37)
HAEGTFTSDVSSYLEGQAAKEFIAWLVKGRG

SEQ ID NO: 6 - protein, 30 aa, Artificial Sequence
  A synthetic modified preselected polypeptide.
HAEGTFTSDVSSYLEGQAAREFIAWLVKGR

SEQ ID NO: 7 - protein, 31 aa, Artificial Sequence
  A synthetic modified preselected polypeptide.
HAEGTFTSDVSSYLEGQAAREFIAWLVKGRG

SEQ ID NO: 8 - protein, 34 aa, Unknown
  GLP-2(1-34).
HADGSFSDGMNTILDNLAARDFINWLIQTKITDR

SEQ ID NO: 9 - protein, 33 aa, Unknown
  GLP-2(1-33).
HADGSFSDGMNTILDNLAARDFINWLIQTKITD

SEQ ID NO: 10 - protein, 33 aa, Artificial Sequence
  A synthetic modified preselected polypeptide.
HGDGSFSDGMNTILDNLAARDFINWLIQTKITD

SEQ ID NO: 11 - protein, 34 aa, Artificial Sequence
  A synthetic modified preselected polypeptide.
HGDGSFSDGMNTILDNLAARDFINWLIQTKITDR

SEQ ID NO: 12 - protein, 44 aa, Unknown
  GRF(1-44).
YADAIFTNSYRKVLGQLSARKLLQDIMSRQQGESNQERGARARL

SEQ ID NO: 13 - protein, 34 aa, Unknown
  PTH(1-34).
SVSEIQLMHNLGKHLNSMERVEWLRKKLQDVHNF

SEQ ID NO: 14 - protein, 37 aa, Unknown
  PTH(1-37).
SVSEIQLMHNLGKHLNSMERVEWLRKKLQDVHNFVAL

SEQ ID NO: 15 - protein, 84 aa, Unknown
  PTH(1-84).
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 16 - protein, 12 aa, Unknown
  Amyloid P Component (27-38).
EKPLQNFTLCFR

SEQ ID NO: 17 - protein, 17 aa, Unknown
  (Tyr0)-Fibrinopeptide A.
YADSGEGDFLAEGGGVR

SEQ ID NO: 18 - protein, 10 aa, Unknown
  Urechistachykinin II.
AAGMGFFGAR

SEQ ID NO: 19 - protein, 17 aa, Unknown
  Amyloid  B-Protein (12-28)
VHHQKLVFFAEDVGSNK

SEQ ID NO: 20 - protein, 14 aa, Unknown
  Amyloid  B-Protein (22-35).
EDVGSNKGAIIGLM

SEQ ID NO: 21 - protein, 29 aa, Camelus
YGFMTSEKSQTPLVTLFKNAIIKNAHKGQ

SEQ ID NO: 22 - protein, 25 aa, Sus scrofa
VEYPVEHPDKFLKFGMTPSKGVLFY

SEQ ID NO: 23 - protein, 21 aa, Mus musculus
CSCNSWLDKECVYFCHLDIIW

SEQ ID NO: 24 - DNA, 90 bp, Unknown
  GLP-1(7-36).
catgctgagggtaccttcacctccgacgtttcctcctacctggaaggtcaggctgctaaagaattcatcgcttggctggttaaaggtcgt

SEQ ID NO: 25 - DNA, 93 bp, Unknown
  GLP-1(7-37).
catgctgagggtaccttcacctccgacgtttcctcctacctggaaggtcaggctgctaaagaattcatcgcttggctggttaaaggtcgtggt

SEQ ID NO: 26 - DNA, 90 bp, Artificial Sequence
  A synthetic modified preselected polypeptide.
catgctgagggtaccttcacctccgacgtttcctcctacctggaaggtcaggctgctcgtgaattcatcgcttggctggttaaaggtcgt

SEQ ID NO: 27 - DNA, 93 bp, Artificial Sequence
  A synthetic modified preselected polypeptide.
catgctgagggtaccttcacctccgacgtttcctcctacctggaaggtcaggctgctcgtgaattcatcgcttggctggttaaaggtcgtggt

SEQ ID NO: 28 - DNA, 102 bp, Unknown
  GLP-2(1-34).
catgctgatggttctttctctgatgagatgaacaccattcttgataatcttgccgcccgtgactttatcaactggttgattcagaccaaaatcactgaccgt

SEQ ID NO: 29 - DNA, 99 bp, Unknown
  GLP-2(1-33).
catgctgatggttctttctctgatgagatgaacaccattcttgataatcttgccgcccgtgactttatcaactggttgattcagaccaaaatcactgac

SEQ ID NO: 30 - DNA, 99 bp, Artificial Sequence
  A synthetic modified preselected polypeptide.
catggtgatggttctttctctgatgagatgaacaccattcttgataatcttgccgcccgtgactttatcaactggttgattcagaccaaaatcactgac

SEQ ID NO: 31 - DNA, 102 bp, Artificial Sequence
  A synthetic modified preselected polypeptide.
catggtgatggttctttctctgatgagatgaacaccattcttgataatcttgccgcccgtgactttatcaactggttgattcagaccaaaatcactgaccgt

SEQ ID NO: 32 - DNA, 132 bp, Unknown
  GRF(1-44).
tacgctgacgctatcttcaccaactcttaccgtaaagttctgggtcagctgtctgctcgtaaactgctgcaggacatcatgtcccgtcagcagggtgaatctaaccaggaacgtggtgctcgtgctcgtctg

SEQ ID NO: 33 - DNA, 102 bp, Unknown
  PTH(1-34).
tctgtttctgaaatccagctgatgcacaacctgggtaaacacctgaactctatggaacgtgttgaatggctgcgtaaaaaactgcaggacgttcacaacttc

SEQ ID NO: 34 - DNA, 111 bp, Unknown
  PTH(1-37).
tctgtttctgaaatccagctgatgcacaacctgggtaaacacctgaactctatggaacgtgttgaatggctgcgtaaaaaactgcaggacgttcacaacttcgttgctctg

SEQ ID NO: 35 - DNA, 252 bp, Unknown
  PTH(1-84).
tctgtttctgaaatccagctgatgcacaacctgggtaaacacctgaactctatggaacgtgttgaatggctgcgtaaaaaactgcaggacgttcacaacttcgttgctctgggtgctccgctggctccgcgtgacgctggttcccagcgtccgcgtaaaaaagaagacaacgttctggttgaatcccacgaaaaatccctgggtgaagctgacaaagctgacgttaacgttctgaccaaagctaaatcccag

SEQ ID NO: 36 - DNA, 36 bp, Unknown
  Amyloid P Component (27-38).
gaaaaaccgctgcagaacttcaccctgtgcttccgt

SEQ ID NO: 37 - DNA, 51 bp, Unknown
  (Tyr0)-Fibrinopeptide A.
tacgctgattccggtgaaggtgatttcctggctgaaggtggtggtgtccgt

SEQ ID NO: 38 - DNA, 30 bp, Unknown
  Urechistachykinin II.
gctgctggtatgggtttcttcggtgcgcgt

SEQ ID NO: 39 - DNA, 51 bp, Unknown
  Amyloid  B-Protein (12-28).
gtccatcatcagaaactggtcttcttcgctgaagatgtcggttccaacaaa

SEQ ID NO: 40 - DNA, 42 bp, Unknown
  Amyloid  B-Protein (22-35).
gaagatgtcggttccaacaaaggtgctattattggtctgatg

SEQ ID NO: 41 - DNA, 93 bp, Camelus
tacggtggtttcatgacctccgaaaaatcccagaccccgctggtcaccctgttcaaaaacgctattattaaaaacgctcataaaaaaggtcag

SEQ ID NO: 42 - DNA, 75 bp, Sus scrofa
gtccagtacccggtcgaacatccggataaattcctgaaattcggtatgaccccgtccaaaggtgtcctgttctac

SEQ ID NO: 43 - DNA, 63 bp, Mus musculus
tgctcctgcaactcctggctggataaagaatgcgtctacttctgccatctggatattatttgg

SEQ ID NO: 44 - protein, 8 aa, Artificial Sequence
  A synthetic cleavable peptide linker.
AFLGPGDR

SEQ ID NO: 45 - protein, 4 aa, Artificial Sequence
  A synthetic cleavable peptide linker.
VDDR

SEQ ID NO: 46 - protein, 4 aa, Artificial Sequence
  A synthetic cleavable peptide linker.
GSDR

SEQ ID NO: 47 - protein, 4 aa, Artificial Sequence
  A synthetic cleavable peptide linker.
ITDR

SEQ ID NO: 48 - protein, 4 aa, Artificial Sequence
  A synthetic cleavable peptide linker.
PGDR

SEQ ID NO: 49 - DNA, 24 bp, Artificial Sequence
  A synthetic cleavable peptide linker.
gctttcctggggccgggtgatcgt

SEQ ID NO: 50 - DNA, 12 bp, Artificial Sequence
  A synthetic cleavable peptide linker.
gtcgacgatcgt

SEQ ID NO: 51 - DNA, 12 bp, Artificial Sequence
  A synthetic cleavable peptide linker.
ggatctgaccgt

SEQ ID NO: 52 - DNA, 12 bp, Artificial Sequence
  A synthetic cleavable peptide linker.
atcactgaccgt

SEQ ID NO: 53 - DNA, 12 bp, Artificial Sequence
  A synthetic cleavable peptide linker.
ccgggtgaccgt

SEQ ID NO: 54 - DNA, 34 bp, Artificial Sequence
  A synthetic primer.
tgcatttctagaattgtgaattgttatccgctca

SEQ ID NO: 55 - DNA, 24 bp, Artificial Sequence
  A synthetic primer.
tcaaagatcttatcgactgcacgg

SEQ ID NO: 56 - DNA, 261 bp, Artificial Sequence
  A synthetic PCR amplification product.
tcaaagatcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaattcacaattctagaaatgca

SEQ ID NO: 57 - DNA, 33 bp, Artificial Sequence
  A synthetic primer.
cctgacgtcccggatgaatgtcagctactgggc

SEQ ID NO: 58 - DNA, 33 bp, Artificial Sequence
  A synthetic primer.
ggctgcgcaaaggagaaaataccgcatcaggaa

SEQ ID NO: 59 - protein, 8 aa, Artificial Sequence
  A synthetic linker.
VDDDDKCH

SEQ ID NO: 60 - DNA, 54 bp, Artificial Sequence
  A synthetic primer.
agaggatccgctgaagaggaggaaattctccttgaagtttccctggtcttcaaa

SEQ ID NO: 61 - DNA, 54 bp, Artificial Sequence
  A synthetic primer.
cagaggtgcgtctggtgcgaattcctttactttgaagaccagggaaacttcaag

SEQ ID NO: 62 - DNA, 54 bp, Artificial Sequence
  A synthetic primer.
gatgtcgacatacgccggaccagtgaacagaggtgcgtctggtgcgaattcctt

SEQ ID NO: 63 - DNA, 111 bp, Artificial Sequence
  A synthetic PCR product.
agaggatccgctgaagaggaggaaattctccttgaagtttccctggtcttcaaagtaaaggaattcgcaccagacgcacctctgttcactggtccggcgtatgtcgacatc

SEQ ID NO: 64 - DNA, 180 bp, Artificial Sequence
  A synthetic PCR product.
gctatggtcgacgacgacgacaaatgccactacgctgacgctatcttcaccaactcttaccgtaaagttctgggtcagctgtctgctcgtaaactgctgcaggacatcatgtcccgtcagcagggtgaatctaaccaggaacgtggtgctcgtgctcgtctgtgccactaactcgagccg

SEQ ID NO: 65 - DNA, 48 bp, Artificial Sequence
  A synthetic primer.
gctatggtcgactgcccaggtatgcatgctgaaggtaccttcacctcc

SEQ ID NO: 66 - DNA, 48 bp, Artificial Sequence
  A synthetic primer.
gttctcgagttaacccggatggcaacgacctttaaccagccaagcgat

SEQ ID NO: 67 - DNA, 48 bp, Artificial Sequence
  A synthetic primer.
tatgtcgacggtggtggtcgtcatgctgaaggtaccttcacctccgac

SEQ ID NO: 68 - DNA, 45 bp, Artificial Sequence
  A synthetic primer.
taactcgagttaagcgaaagcacgacctttaaccagccaagcgat

SEQ ID NO: 69 - DNA, 190 bp, Artificial Sequence
  A synthetic PCR product.
gttcgaagatctaattcccggggatcaggcctcgcttataaatatggtattgatgtacttgccggtgtgattggctcagattacagaggagagttgaaagcaatccttgacaatactacagaacgtgactataatatcaaaaaagtcgacgaggaattgtgagcggataacaattcacaattctagaaat

SEQ ID NO: 70 - DNA, 54 bp, Artificial Sequence
  A synthetic primer.
atggctagcatgactggtggacagcaaatgggtaaaggatccgctgaagaggag

SEQ ID NO: 71 - DNA, 45 bp, Artificial Sequence
  A synthetic primer.
tatcactcgagattagtcgacatacgccggaccagtgaacagagg

SEQ ID NO: 72 - DNA, 45 bp, Artificial Sequence
  A synthetic primer.
atggctagccatatgcatgctgaaggtaccttcacctccgacgtt

SEQ ID NO: 73 - DNA, 54 bp, Artificial Sequence
  A synthetic primer.
catgctagccatacctggaccaccaccagcgaaagcacgacctttaaccagcca

SEQ ID NO: 74 - DNA, 129 bp, Artificial Sequence
  A synthetic PCR product.
atggtcgacgatcgtcatggtgatggttctttctctgatgagatgaacaccattcttgataatcttgccgcccgtgactttatcaactggttgattcagaccaaaatcactgactaataactcgaggaa

SEQ ID NO: 75 - DNA, 276 bp, Artificial Sequence
  A synthetic PCR product.
ataccacatatgtctgtttctgaaatccagctgatgcacaacctgggtaaacacctgaactctatggaacgtgttgaatggctgcgtaaaaaactgcaggacgttcacaacttcgttgctctgggtgctccgctggctccgcgtgacgctggttcccagcgtccgcgtaaaaaagaagacaacgttctggttgaatcccacgaaaaatccctgggtgaagctgacaaagctgacgttaacgttctgaccaaagctaaatcccagtaactcgagtat

SEQ ID NO: 76 - DNA, 291 bp, Artificial Sequence
  A synthetic PCR product.
tatggattcgacgacgacaaatgccactctgtttctgaaatccagctgatgcacaacctgggtaaacacctgaactctatggaacgtgttgaatggctgcgtaaaaaactgcaggacgttcacaacttcgttgctctgggtgctccgctggctccgcgtgacgctggttcccagcgtccgcgtaaaaaagaagacaacgttctggttgaatcccacgaaaaatccctgggtgaagctgacaaagctgacgttaacgttctgaccaaagctaaatcccagtaactcgagtat

SEQ ID NO: 77 - DNA, 291 bp, Artificial Sequence
  A synthetic PCR product.
tatgtcgacgacgacgacaaatgccactctgtttctgaaatccagctgatgcacaacctgggtaaacacctgaactctatggaacgtgttgaatggctgcgtaaaaaactgcaggacgttcacaacttcgttgctctgggtgctccgctggctccgcgtgacgctggttcccagcgtccgcgtaaaaaagaagacaacgttctggttgaatcccacgaaaaatccctgggtgaagctgacaaagctgacgttaacgttctgaccaaagctaaatcccagtaactcgagtat

SEQ ID NO: 78 - protein, 99 aa, Artificial Sequence
  A synthetic pBN121-T7tagPh-CH-GRF(1-44)CH.
MASMTGGQQMGRGSAEEEEILLEVSLVFKVKEFAPDAPLFTGPAYVDDDDKCHYADAIFTNSYRKVLGQLSARKLLQDIMSRQQGESNQERGARARLCH

SEQ ID NO: 79 - DNA, 306 bp, Artificial Sequence
  A synthetic pBN121-T7tagPh-CH-GRF(1-44)CH.
atggctagcatgactggtggacagcaaatgggtcgcggatccgctgaagaggaggaaattctccttgaagtttccctggtcttcaaagtaaaggaattcgcaccagacgcacctctgttcactggtccggcgtatgtcgacgacgacgacaaatgccattacgctgacgctatcttcaccaactcttaccgtaaagttctgggtcagctgtctgctcgtaaactgctgcaggacatcatgtcccgtcagcagggtgaatctaaccaggaacgtggtgctcgtgctcgtctgtgccactaactcgag

SEQ ID NO: 80 - protein, 85 aa, Artificial Sequence
  A synthetic pBN121-T7tagPh-CPGM-GLP-1(7-36) CHPG.
MASMTGGQQMGRGSAEEEEILLEVSLVFKVKEFAPDAPLFTGPAYVDCPGMHAEGTFTSDVSSYLEGQAAKEFIAWLVKGRCHPG

SEQ ID NO: 81 - DNA, 265 bp, Artificial Sequence
  A synthetic BN121-T7tagPh-CPGM-GLP-1(7-36) CHPG.
atggctagcatgactggtggacagcaaatgggtcgcggatccgctgaagaggaggaaattctccttgaagtttccctggtcttcaaagtaaaggaattcgcaccagacgcacctctgttcactggtccggcgtatgtcgactgcccaggtatgcatgctgaaggtaccttcacctccgacgtttcctcctacctggaaggtcaggctgctaaagaattcatcgcttggctggttaaaggtcgttgccatccgggttaactcgagg

SEQ ID NO: 82 - protein, 84 aa, Artificial Sequence
  A synthetic pBN121-T7tagPh-GGGR-GLP-1(7-36)AFA.
MASMTGGQQMGRGSAEEEEILLEVSLVFKVKEFAPDAPLFTGPAYVDGGGRHAEGTFTSDVSSYLEGQAAKEFIAWLVKGRAFA

SEQ ID NO: 83 - DNA, 261 bp, Artificial Sequence
  A synthetic N121-T7tagPh-GGGR-GLP-1(7-36)AFA.
atggctagcatgactggtggacagcaaatgggtcgcggatccgctgaagaggaggaaattctccttgaagtttccctggtcttcaaagtaaaggaattcgcaccagacgcacctctgttcactggtccggcgtatgtcgacggtggtggtcgtcatgctgaaggtaccttcacctccgacgtttcctcctacctggaaggtcaggctgctaaagaattcatcgcttggctggttaaaggtcgtgctttcgcttaactcgag

SEQ ID NO: 84 - protein, 86 aa, Artificial Sequence
  A synthetic pBN122-M-GLP-1(7-36) AFAFGGGPG-T7tagPh.
MHAEGTFTSDVSSYLEGQAAKEFIAWLVKGRAFAGGGPGMASMTGGQQMGKGSAEEEEILLEVSLVFKVKEFAPDAPLFTGPAYVD

SEQ ID NO: 85 - DNA, 267 bp, Artificial Sequence
  A synthetic pBN122-M-GLP-1(7-36) AFAFGGGPG-T7tagPh.
atgcatgctgaaggtaccttcacctccgacgtttcctcctacctggaaggtcaggctgctaaagaattcatcgcttggctggttaaaggtcgtgctttcgctggtggtggtccaggtatggctagcatgactggtggacagcaaatgggtaaaggatccgctgaagaggaggaaattctccttgaagtttccctggtcttcaaagtaaaggaattcgcaccagacgcacctctgttcactggtccggcgtatgtcgactaactcgag

SEQ ID NO: 86 - protein, 82 aa, Artificial Sequence
  A synthetic pBN121-T7tagPh-VDDR-GLP-2(1-33)A2G.
MASMTGGQQMGRGSAEEEEILLEVSLVFKVKEFAPDAPLFTGPAYVDDRHGDGSFSDEMNTILDNLAARDFINWLIQTKITD

SEQ ID NO: 87 - DNA, 258 bp, Artificial Sequence
  A synthetic pBN121-T7tagPh-VDDR-GLP-2(1-33)A2G.
atggctagcatgactggtggacagcaaatgggtcgcggatccgctgaagaggaggaaattctccttgaagtttccctggtcttcaaagtaaaggaattcgcaccagacgcacctctgttcactggtccggcgtatgtcgacgatcgtcatggtgatggttctttctctgatgagatgaacaccattcttgataatcttgccgcccgtgactttatcaactggttgattcagaccaaaatcactgactaataactcgag

SEQ ID NO: 88 - protein, 85 aa, Artificial Sequence
  A synthetic pBN121-M-PTH(1-84).
MSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 89 - DNA, 264 bp, Artificial Sequence
  A synthetic pBN121-M-PTH(1-84).
atgtctgtttctgaaatccagctgatgcacaacctgggtaaacacctgaactctatggaacgtgttgaatggctgcgtaaaaaactgcaggacgttcacaacttcgttgctctgggtgctccgctggctccgcgtgacgctggttcccagcgtccgcgtaaaaaagaagacaacgttctggttgaatcccacgaaaaatccctgggtgaagctgacaaagctgacgttaacgttctgaccaaagctaaatcccagtaactcgag

SEQ ID NO: 90 - protein, 104 aa, Artificial Sequence
  A synthetic pBN121-T7tag-CH-PTH(1-84).
MASMTGGQQMGRGSDDDKCHSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 91 - DNA, 321 bp, Artificial Sequence
  A synthetic pBN121-T7tag-CH-PTH(1-84).
atggctagcatgactggtggacagcaaatgggtcgcggatccgacgacgacaaatgccactctgtttctgaaatccagctgatgcacaacctgggtaaacacctgaactctatggaacgtgttgaatggctgcgtaaaaaactgcaggacgttcacaacttcgttgctctgggtgctccgctggctccgcgtgacgctggttcccagcgtccgcgtaaaaaagaagacaacgttctggttgaatcccacgaaaaatccctgggtgaagctgacaaagctgacgttaacgttctgaccaaagctaaatcccagtaactcgag

SEQ ID NO: 92 - protein, 137 aa, Artificial Sequence
  A synthetic pBN121-T7tagPh-CH-PTH(1-84).
MASMTGGQQMGRGSAEEEEILLEVSLVFKVKEFAPDAPLFTGPAYVDDDDKCHSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 93 - DNA, 420 bp, Artificial Sequence
  A synthetic pBN121-T7tagPh-CH-PTH(1-84).
atggctagcatgactggtggacagcaaatgggtcgcggatccgctgaagaggaggaaattctccttgaagtttccctggtcttcaaagtaaaggaattcgcaccagacgcacctctgttcactggtccggcgtatgtcgacgacgacgacaaatgccactctgtttctgaaatccagctgatgcacaacctgggtaaacacctgaactctatggaacgtgttgaatggctgcgtaaaaaactgcaggacgttcacaacttcgttgctctgggtgctccgctggctccgcgtgacgctggttcccagcgtccgcgtaaaaaagaagacaacgttctggttgaatcccacgaaaaatccctgggtgaagctgacaaagctgacgttaacgttctgaccaaagctaaatcccagtaactcgag

(19) United States
(12) Patent Application Publication
(10) Pub. No.: US 2005/0239172 A1
(43) Pub. Date: Oct. 27, 2005

(54) Methods and DNA constructs for high yield production of polypeptides

(76) Inventors: Fred W. Wagner, Walton, NE (US); Peng Luan, Fishers, IN (US); Yuannan Xia, Lincoln, NE (US); Barton Holmquist, Eagle, NE (US)

Correspondence Address: Schwegman, Lundberg, Woessner & Kluth, P.A., P.O. Box 2938, Minneapolis, MN, 55402, US

(21) Appl. No.: 10/997,700

(22) Filed: Nov. 24, 2004

Related U.S. Application Data
(63) Continuation of application No. PCT/US03/16645, filed on May 23, 2003.
(60) Provisional application No. 60/383,212, filed on May 24, 2002.

(51) Int. Cl. C12P 21/04; C07H 21/04; C07K 14/475

(52) U.S. Cl. 435/69.7; 435/320.1; 435/471; 435/325; 435/252.3; 530/350; 536/23.5

(57) ABSTRACT

The invention provides an inclusion body fusion partner to increase peptide and polypeptide production in a cell.

### FIELD OF THE INVENTION

The present invention relates generally to the field of protein expression. More specifically, it relates to methods and DNA constructs for the expression of polypeptides and proteins.

### BACKGROUND OF THE INVENTION

Polypeptides are useful for the treatment of disease in humans and animals. Examples of such polypeptides include insulin for the treatment of diabetes, interferon for treating viral infections, interleukins for modulating the immune system, erythropoietin for stimulating red blood cell formation, and growth factors that act to mediate both prenatal and postnatal growth.

Many bioactive polypeptides can be produced through use of chemical synthesis methods. However, such production methods are often times inefficient and labor intensive which leads to increased cost and lessened availability of therapeutically useful polypeptides. An alternative to chemical synthesis is provided by recombinant technology which allows the high yield production of bioactive polypeptides in microbes. Such production permits a greater number of people to be treated at a lowered cost.

While great strides have been made in recombinant technology, expression of proteins and peptides in cells can be problematic. This can be due to low expression levels or through destruction of the expressed polypeptide by proteolytic enzymes contained within the cells. This is especially problematic when short proteins and peptides are being expressed.

These problems have been addressed in the past by producing fusion proteins that contain the desired polypeptide fused to a carrier polypeptide. Expression of a desired polypeptide as a fusion protein in a cell will often times protect the desired polypeptide from destructive enzymes and allow the fusion protein to be purified in high yields. The fusion protein is then treated to cleave the desired polypeptide from the carrier polypeptide and the desired polypeptide is isolated. Many carrier polypeptides have been used according to this protocol. Examples of such carrier polypeptides include β-galactosidase, glutathione-S-transferase, the N-terminus of L-ribulokinase, bacteriophage T4 gp55 protein, and bacterial ketosterioid isomerase protein. While this protocol offers many advantages, it suffers from decreased production efficiency due to the large size of the carrier protein. Thus, the desired polypeptide may make up a small percentage of the total mass of the purified fusion protein resulting in decreased yields of the desired polypeptide.

Another method to produce a desired polypeptide through recombinant technology involves producing a fusion protein that contains the desired polypeptide fused to an additional polypeptide sequence. In this case, the additional polypeptide sequence causes the fusion protein to form an insoluble mass in a cell called an inclusion body. These inclusion bodies are then isolated from the cell and the fusion protein is purified. The fusion protein is then treated to cleave the additional polypeptide sequence from the fusion protein and the desired polypeptide is isolated. This method has provided high level of expression of desired polypeptides. An advantage of such a method is that the additional polypeptide sequence will often times be smaller than the desired polypeptide and will therefore constitute a smaller percentage of the fusion protein produced leading to increased production efficiency. A disadvantage of such systems is that they produce inclusion bodies that are very difficult to solubilize in order to isolate a polypeptide of interest.

Accordingly, a need exists for additional polypeptide sequences that may be used to produce desired polypeptides through formation of inclusion bodies. A need also exists for additional polypeptide sequences that may be used to produce inclusion bodies having characteristics that allow them to be more easily manipulated during the production and purification of desired polypeptides.

### SUMMARY OF THE INVENTION

The invention provides an expression cassette for the expression of a tandem polypeptide that forms an inclusion body. The invention also provides an expression cassette for the expression of a tandem polypeptide that forms an inclusion body having isolation enhancement. Also provided by the invention is an RNA produced by transcription of an expression cassette of the invention. The invention also provides a protein produced by translation of an RNA produced by transcription of an expression cassette of the invention. Also provided by the invention is a nucleic acid construct containing a vector an expression cassette of the invention. The invention also provides a cell containing an expression cassette or a nucleic acid construct of the invention. Also provided by the invention is a tandem polypeptide containing an inclusion body fusion partner operably linked to a preselected polypeptide. The invention also provides a method to select an inclusion body fusion partner that confers isolation enhancement to an inclusion body.

An expression cassette can encode a tandem polypeptide that includes a preselected polypeptide that is operably linked to an inclusion body fusion partner. An expression cassette can encode a tandem polypeptide that includes a preselected polypeptide that is operably linked to an inclusion body fusion partner and a cleavable peptide linker. An expression cassette can also encode a tandem polypeptide that includes a preselected polypeptide that is operably linked to an inclusion body fusion partner and a fusion tag. An expression cassette can also encode a tandem polypeptide that includes a preselected polypeptide that is operably linked to an inclusion body fusion partner, a cleavable linker peptide, and a fusion tag. An expression cassette can encode a tandem polypeptide having a preselected polypeptide, an inclusion body fusion partner, a cleavable peptide linker, and a fusion tag operably linked in any order that will cause the tandem polypeptide to form an inclusion body.

Preferably, an expression cassette encodes a preselected polypeptide that is a bioactive polypeptide. More preferably an expression cassette encodes a preselected polypeptide that is useful to treat a disease in a human or animal. Even more preferably an expression cassette encodes a preselected polypeptide that is glucagon-like peptide-1 (GLP-1), glucagon-like peptide-2 (GLP-2), parathyroid hormone (PTH), or a growth hormone releasing factor (GRF). Preferably an expression cassette encodes a preselected polypeptide that is a protease. More preferably an expression cassette encodes a preselected polypeptide that is clostripain. An expression cassette can encode more than one copy of a preselected polypeptide. Preferably an expression cassette encodes twenty copies of a preselected polypeptide. More preferably an expression cassette encodes ten copies of a preselected polypeptide. Even more preferably an expression cassette encodes five copies of a preselected polypeptide. Still even more preferably an expression cassette encodes two copies of a preselected polypeptide. Most preferably an expression cassette encodes one copy of a preselected polypeptide.

Preferably an expression cassette encodes an inclusion body fusion partner having an amino acid sequence that is a variant of SEQ ID NO: 1. More preferably an expression cassette encodes an inclusion body fusion partner having SEQ ID NO: 1. Preferably an expression cassette encodes an inclusion body fusion partner that confers isolation enhancement to the inclusion body formed from the tandem polypeptide. More preferably an expression cassette encodes an inclusion body fusion partner that confers protease resistance, controllable solubility, purification stability, or self-adhesion to an inclusion body formed from a tandem polypeptide. An expression cassette can encode an inclusion body fusion partner that can be operably linked to a preselected polypeptide at the amino-terminus of the preselected polypeptide, the carboxyl-terminus of the preselected polypeptide, or the amino-terminus and the carboxyl-terminus of the preselected polypeptide. Preferably an expression cassette encodes an inclusion body fusion partner that is independently operably linked to each of the amino-terminus and the carboxyl-terminus of a preselected polypeptide. More preferably an expression cassette encodes an inclusion body fusion partner that is operably linked to the carboxyl-terminus of a preselected polypeptide. Even more preferably an expression cassette encodes an inclusion body fusion partner that is operably linked to the amino-terminus of a preselected polypeptide. An expression cassette can encode one or more inclusion body fusion partners that can be operably linked to the amino-terminus, the carboxyl-terminus or the amino-terminus and the carboxyl-terminus of a preselected polypeptide. Preferably an expression cassette encodes twenty inclusion body fusion partners that are operably linked to the preselected polypeptide. More preferably an expression cassette encodes ten inclusion body fusion partners that are linked to the preselected polypeptide. Even more preferably an expression cassette encodes five inclusion body fusion partners that are linked to the preselected polypeptide. Still even more preferably an expression cassette encodes two inclusion body fusion partners that are linked to the preselected polypeptide. Most preferably an expression cassette encodes one inclusion body fusion partner that is linked to the preselected polypeptide.

Preferably an expression cassette encodes a fusion tag that increases the ease with which an operably linked tandem polypeptide can be isolated. More preferably an expression cassette encodes a fusion tag that is a poly-histidine tag. More preferably an expression cassette encodes a fusion tag that is an epitope tag. Even more preferably an expression cassette encodes a fusion tag that is a substrate binding tag. Still even more preferably an expression cassette encodes a fusion tag that is glutathione-S-transferase or arabinose binding protein. An expression cassette can encode a fusion tag that is a ligand for a cellular receptor. Preferably an expression cassette encodes a fusion tag that is a ligand for an insulin receptor.

An expression cassette of the invention can encode one or more cleavable peptide linkers that are operably linked to an inclusion body fusion partner and a preselected polypeptide. An expression cassette of the invention can also encode one or more cleavable peptide linkers that are operably linked to an inclusion body fusion partner, a preselected polypeptide and a fusion tag. Preferably an expression cassette encodes a tandem polypeptide having twenty cleavable peptide linkers. More preferably an expression cassette encodes a tandem polypeptide having ten cleavable peptide linkers. Even more preferably an expression cassette encodes a tandem polypeptide having five cleavable peptide linkers. Most preferably an expression cassette encodes a tandem polypeptide having a cleavable peptide linker independently positioned, between an inclusion body fusion partner and a preselected polypeptide, between an inclusion body fusion partner and a fusion tag, between two preselected polypeptides, or between a preselected polypeptide and a fusion tag.

An expression cassette can encode a cleavable peptide linker that may be cleaved with a chemical agent. Preferably an expression cassette encodes a cleavable peptide linker that is cleavable with cyanogen bromide. More preferably an expression cassette encodes a cleavable peptide linker that is cleavable with palladium. An expression cassette can encode a cleavable peptide linker that may be cleaved with a protease. Preferably an expression cassette encodes a cleavable peptide linker that is cleavable with a tissue specific protease. More preferably an expression cassette encodes a cleavable peptide linker that is cleavable with a serine protease, an aspartic protease, a cysteine protease, or a metalloprotease. Most preferably an expression cassette encodes a cleavable peptide linker that is cleavable with clostripain.

An expression cassette of the invention includes a promoter. Preferably the promoter is a constituitive promoter. More preferably the promoter is a regulatable promoter. Most preferably the promoter is an inducible promoter.

An expression cassette of the invention may include one or more suppressible stop codons. Preferably a suppressible stop codon is an amber or an ochre stop codon.

An expression cassette of the invention may encode a fusion tag. An expression cassette can encode a fusion tag that may be a ligand binding domain. Preferably an expression cassette encodes a fusion tag that is a metal binding domain. More preferably an expression cassette encodes a fusion tag that is a sugar binding domain. Even more preferably an expression cassette encodes a fusion tag that is a peptide binding domain. Most preferably an expression cassette encodes a fusion tag that is an amino acid binding domain. An expression cassette can encode a fusion tag that may be an antibody epitope. Preferably an expression cassette encodes a fusion tag that is recognized by an anti-maltose binding protein antibody. More preferably an expression cassette encodes a fusion tag that is recognized by an anti-T7 gene 10 bacteriophage antibody. An expression cassette can encode a fusion tag that may be a fluorescent protein. Preferably an expression cassette encodes a fusion tag that is a green fluorescent protein, a yellow fluorescent protein, a red fluorescent protein or a cayenne fluorescent protein.

The invention provides a nucleic acid construct containing a vector and an expression cassette of the invention. Preferably the vector is a plasmid, phagemid, cosmid, F-factor, virus, bacteriophage, yeast artificial chromosome, or bacterial artificial chromosome. Preferably the nucleic acid construct is RNA. More preferably the nucleic acid construct is DNA.

The invention provides a cell containing a nucleic acid construct of the invention. Preferably the cell is a eulcaryotic cell. More preferably the eukaryotic cell is a mammalian cell. Even more preferably the eukaryotic cell is a yeast cell. Most preferably the eukaryotic cell is an insect cell. More preferably the cell is a prokaryotic cell. Even more preferably the prokaryotic cell is a bacterium. Still even more preferably the prokaryotic cell is an Escherichia coli. Most preferably the prokaryotic cell is Escherichia coli BL21.

The invention provides a tandem polypeptide that includes a preselected polypeptide that is operably linked to an inclusion body fusion partner. The invention also provides a tandem polypeptide that includes a preselected polypeptide that is operably linked to an inclusion body fusion partner and a cleavable peptide linker. The invention also provides a tandem polypeptide that includes a preselected polypeptide that is operably linked to an inclusion body fusion partner, and a fusion tag. The invention also provides a tandem polypeptide that includes a preselected polypeptide that is operably linked to an inclusion body fusion partner, a cleavable linker peptide, and a fusion tag. The invention also provides a tandem polypeptide that includes a preselected polypeptide that is operably linked to an inclusion body fusion partner, and independently operably linked to one or more cleavable peptide linkers, or to one or more fusion tags in any order that will cause a tandem polypeptide to form an inclusion body.

The invention also provides a method to select an inclusion body fusion partner that confers isolation enhancement to an inclusion body. Preferably the isolation enhancement is altered isoelectric point. More preferably the isolation enhancement is protease resistance. Even more preferably the isolation enhancement is increased solubility. Still even more preferably the isolation enhancement is self-adhesion. Most preferably the isolation enhancement is purification stability.

Definitions

Abbreviations: IPTG: isopropylthio-β-D-galactoside; PCR: polymerase chain reaction; mRNA: messenger ribonucleic acid; DNA: deoxyribonucleic acid; RNA: ribonucleic acid; β-gal: β-galactosidase; GST: glutathione-S-transferase; CAT: chloramphenicol acetyl transferase; SPA: staphylococcal protein A; SPG: streptococcal protein G; MBP: maltose binding protein; SBD: starch binding protein; CBD_{CenA}: cellulose-binding domain of endoglucanaase A; CBD_{Cex}: cellulose binding domain of exoglucanase Cex; FLAG: hydrophilic 8-amino acid peptide; TrpE: tryptophan synthase; GLP-1: glucagon-like peptide-1; GLP-2: glucagone-like peptide-2; PTH: parathyroid hormone; GRF: growth hormone releasing factor.

The term “Altered isoelectric point” refers to changing the amino acid composition of an inclusion body fission partner to effect a change in the isoelectric point of a tandem polypeptide that includes the inclusion body fusion partner operably linked to a preselected polypeptide.

An “Amino acid analog” includes amino acids that are in the D rather than L form, as well as other well known amino acid analogs, e.g., N-alkyl amino acids, lactic acid, and the like. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, N-methyl-alanine, para-benzoyl-phenylalanine, phenylglycine, propargylglycine, sarcosine, N-acetylserine, N-formylmethionine, 3-methylhistidine, 5-hydroxylysine, norleucine, norvaline, orthonitrophenylglycine and other similar amino acids.

The terms, “cells,” “cell cultures”, “Recombinant host cells”, “host cells”, and other such terms denote, for example, microorganisms, insect cells, and mammalian cells, that can be, or have been, used as recipients for nucleic acid constructs or expression cassettes, and include the progeny of the original cell which has been transformed. It is understood that the progeny of a single parental cell may not necessarily be completely identical in morphology or in genomic or total DNA complement as the original parent, due to natural, accidental, or deliberate mutation. Many cells are available from ATCC and commercial sources. Many mammalian cell lines are known in the art and include, but are not limited to, Chinese hamster ovary (CHO) cells, HeLa cells, baby hamster kidney (BHK) cells, monkey kidney cells (COS), and human hepatocellular carcinoma cells (e.g., Hep G2). Many prokaryotic cells are known in the art and include, but are not limited to, Escherichia coli and. Salmonella typhimurium. Sambrook and Russell, Molecular Cloning: A Laboratory Manual, 3rd edition (Jan. 15, 2001) Cold Spring Harbor Laboratory Press, ISBN: 0879695765. Many insect cells are known in the art and include, but are not limited to, silkworm cells and mosquito cells. (Franke and Hruby, J. Gen. Virol., 66:2761 (1985); Marumoto et al., J. Gen. Virol., 68:2599 (1987)).

A “Cleavable peptide linker” refers to a peptide sequence having a cleavage recognition sequence. A cleavable peptide linker can be cleaved by an enzymatic or a chemical cleavage agent. Numerous peptide sequences are known that are cleaved by enzymes or chemicals. Harlow and Lane, Antibodies: A Laboratory Manual, Cold Spring Harbor Laboratory Press, Cold Spring Harbor, N.Y. (1988); Walsh, Proteins Biochemistry and Biotechnology, John Wiley & Sons, LTD., West Sussex, England (2002).

A “Cleavage agent” is a chemical or enzyme that recognizes a cleavage site in a polypeptide and causes the polypeptide to be split into two polypeptides through breakage of a bond within the polypeptide. Examples of cleavage agents include, but are not limited to, chemicals and proteases.

A “Coding sequence” is a nucleic acid sequence that is translated into a polypeptide, such as a preselected polypeptide, usually via mRNA. The boundaries of the coding sequence are determined by a translation start codon at the 5′-terminus and a translation stop codon at the 3′-terminus of an mRNA. A coding sequence can include, but is not limited to, cDNA, and recombinant nucleic acid sequences.

A “Conservative amino acid” refers to an amino acid that is functionally similar to a second amino acid. Such amino acids may be substituted for each other in a polypeptide with a minimal disturbance to the structure or function of the polypeptide according to well known techniques. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q).

“Constitutive promoter” refers to a promoter that is able to express a gene or open reading frame without additional regulation. Such constitutive promoters provide constant expression of operatively linked genes or open reading frames under nearly all conditions.

A “Fusion tag” is an amino acid segment that can be operably linked to a tandem polypeptide that contains an inclusion body fusion partner operably linked to a preselected amino acid sequence. A fusion tag may exhibit numerous properties. For example, the fusion tag may selectively bind to purification media that contains a binding partner for the fusion tag and allow the operably linked tandem polypeptide to be easily purified. Such fusion tags may include, but are not limited to, glutathione-S-transferase, polyhistidine, maltose binding protein, avidin, biotin, or streptavidin. In another example, a fusion tag may be a ligand for a cellular receptor, such as an insulin receptor. This interaction will allow a tandem polypeptide that is operably linked to the fusion tag to be specifically targeted to a specific cell type based on the receptor expressed by the cell. In another example, the fusion tag may be a polypeptide that serves to label the operably linked tandem polypeptide. Examples of such fusion tags include, but are not limited to, green fluorescent protein, red fluorescent protein, yellow fluorescent protein, cayenne fluorescent protein.

The term “Gene” is used broadly to refer to any segment of nucleic acid that encodes a preselected polypeptide. Thus, a gene may include a coding sequence for a preselected polypeptide and/or the regulatory sequences required for expression. A gene can be obtained from a variety of sources, including being cloned from a source of interest or by being synthesized from known or predicted sequence information. A gene of the invention may also be optimized for expression in a given organism. For example, a codon usage table may be used to optimize a gene for expression in Escherichia coli. Harlow and Lane, Antibodies: A Laboratory Manual, Cold Spring Harbor Laboratory Press, Cold Spring Harbor, N.Y. (1988).

An “Inclusion body” is an amorphous deposit in the cytoplasm of a cell; an aggregated protein appropriate to the cell but damaged, improperly folded or liganded, or a similarly inappropriately processed foreign protein, such as a viral coat protein or recombinant DNA product.

An “Inclusion body fusion partner” is an amino acid sequence having SEQ ID NO: 1, or a variant thereof, that causes a tandem polypeptide containing a preselected polypeptide and an inclusion body fusion partner to form an inclusion body when expressed within a cell. The inclusion body fusion partners of the invention can be altered to confer isolation enhancement onto an inclusion body that contains the altered inclusion body fusion partner.

“Inducible promoter” refers to those regulated promoters that can be turned on by an external stimulus (e.g. a chemical, nutritional stress, or heat). For example, the lac promoter can be induced through use of IPTG (isopropylthio-β-D-galactoside). In another example, the bacteriophage lambda P_{L} promoter can be regulated by the temperature-sensitive repressor, cIts857 which represses P_{L} transcription at low temperatures but not at high temperatures. Thus, temperature shift may be used to induce transcription from the P_{L} promoter. Sambrook and Russell, Molecular Cloning: A Laboratory Manual, 3rd edition (Jan. 15, 2001) Cold Spring Harbor Laboratory Press, ISBN: 0879695765.

The term “Isolation enhancement” refers to the alteration of characteristics of an inclusion body that aids in purification of tandem polypeptides that compose the inclusion body. For example, alteration of an inclusion body fusion partner to increase the solubility of an inclusion body formed from tandem polypeptides that include the altered inclusion body fusion partner would be isolation enhancement. In another example, alteration of an inclusion body fusion partner to control the solubility of an inclusion body at a select pH would be isolation enhancement.

An “open reading frame” (ORF) is a region of a nucleic acid sequence which encodes a polypeptide, such as a preselected polypeptide; this region may represent a portion of a coding sequence or a total coding sequence. “Operably-linked” refers to the association of nucleic acid sequences or amino acid sequences on a single nucleic acid fragment or a single amino acid sequence so that the function of one is affected by the other. For example, a regulatory DNA sequence is said to be “operably linked to” or “associated with” a DNA sequence that codes for an RNA if the two sequences are situated such that the regulatory DNA sequence affects expression of the coding DNA sequence (i.e., that the coding sequence or functional RNA is under the transcriptional control of the promoter). In an example related to amino acid sequences, an inclusion body fusion partner is said to be operably linked to a preselected amino acid sequence when the inclusion body fusion partner causes a tandem polypeptide to form an inclusion body. In another example, a signal sequence is said to be operably linked to a preselected amino acid when the signal sequence directs the tandem polypeptide to a specific location in a cell.

An “Operator” is a site on DNA at which a repressor protein binds to prevent transcription from initiating at the adjacent promoter. Many operators and repressors are known and may be exemplified by the lac operator and the lac repressor. Lewin, Genes VII, Oxford University Press, New York, N.Y. (2000).

The term “polypeptide” refers to a polymer of amino acids, thus, peptides, oligopeptides, and proteins are included within the definition of polypeptide. This term optionally includes post expression modifications of the polypeptide, for example, glycosylations, acetylations, phosphorylations and the like. Included within the definition are, for example, polypeptides containing one or more analogues of an amino acid or labeled amino acids. Examples of rabiolabeled amino acids include, but are not limited to, S³⁵-methionine, S³⁵-cysteine, H³-alanine, and the like. The invention may also be used to produce deuterated polypeptides by growing cells that express the polypeptide in deuterium. Such deuterated polypeptides are particularly useful during NMR studies. “Promoter” refers to a nucleotide sequence, usually upstream (5′) to its coding sequence, which controls the expression of the coding sequence by providing the recognition site for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or environmental conditions.

The term “Purification stability” refers to the isolation characteristics of an inclusion body formed from a tandem polypeptide having an inclusion body fusion partner operably linked to a preselected polypeptide. High purification stability indicates that an inclusion body is able to be isolated from a cell in which it was produced. Low purification stability indicates that the inclusion body is unstable during purification due to dissociation of the tandem polypeptides forming the inclusion body.

“Purified” and “isolated” mean, when referring to a polypeptide or nucleic acid sequence, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type present (but water, buffers, and other small molecules, especially molecules having a molecular weight of less than 1000, can be present).

“Regulated promoter” refers to a promoter that directs gene expression in a controlled manner rather than in a constitutive manner. Regulated promoters include inducible promoters and repressable promoters. Such promoters may include natural and synthetic sequences as well as sequences that may be a combination of synthetic and natural sequences. Different promoters may direct the expression of a gene in response to different environmental conditions. Typical regulated promoters useful in the invention include, but are not limited to, promoters used to regulate metabolism (e.g. an IPTG-inducible lac promoter) heat-shock promoters (e.g. an SOS promoter), and bacteriophage promoters (e.g. a T7 promoter).

A “Ribosome binding site” is a DNA sequence that encodes a site on an mRNA at which the small and large subunits of a ribosome associate to form an intact ribosome and initiate translation of the mRNA. Ribosome binding site consensus sequences include AGGA or GAGG and are usually located some 8 to 13 nucleotides upstream (5′) of the initiator AUG codon on the mRNA. Many ribosome binding sites are known in the art. (Shine et al., Nature, 254: 34 (1975); Steitz et al., “Genetic signals and nucleotide sequences in messenger RNA”, in: Biological Regulation and Development: Gene Expression (ed. R. F. Goldberger) (1979)).

The term “Self-adhesion” refers to the association between individual tandem polypeptides, having an inclusion body fusion partner operably linked to a preselected polypeptide sequence, to form an inclusion body. Self-adhesion affects the purification stability of an inclusion body formed from a tandem polypeptide. Self-adhesion that is too great produces inclusion bodies having tandem polypeptides that are so tightly associated with each other that it is difficult to separate individual tandem polypeptides from an isolated inclusion body. Self-adhesion that is too low produces inclusion bodies that are unstable during isolation due to dissociation of the tandem polypeptides that form the inclusion body. Self-adhesion can be regulated by altering the amino acid sequence of an inclusion body fusion partner.

A “Signal sequence” is a region in a protein or polypeptide responsible for directing an operably linked polypeptide to a cellular location, compartment, or secretion from the cell as designated by the signal sequence. For example, signal sequences direct operably linked polypeptides to the inner membrane, periplasmic space, and outer membrane in bacteria. The nucleic acid and amino acid sequences of such signal sequences are well known in the art and have been reported. Watson, Molecular Biology of the Gene, 4th edition, Benjamin/Cummings Publishing Company, Inc., Menlo Park, Calif. (1987); Masui et al., in: Experimental Manipulation of Gene Expression, (1983); Ghrayeb et al., EMBO J., 3: 2437 (1984); Oka et al., Proc. Natl. Acad. Sci. USA, 82: 7212 (1985); Palva et al., Proc. Natl. Acad. Sci. USA, 79: 5582 (1982); U.S. Pat. No. 4,336,336).

Signal sequences, preferably for use in insect cells, can be derived from genes for secreted insect or baculovirus proteins, such as the baculovirus polyhedrin gene (Carbonell et al., Gene 73: 409 (1988)). Alternatively, since the signals for mammalian cell posttranslational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, signal sequences of non-insect origin, such as those derived from genes encoding human α-interferon (Maeda et al., Nature, 315:592 (1985)), human gastrin-releasing peptide (Lebacq-Verheyden et al., Mol. Cell. Biol., 8: 3129 (1988)), human IL-2 (Smith et al., Proc. Natl. Acad. Sci. USA, 82: 8404 (1985)), mouse IL-3 (Miyajima et al., Gene 58: 273 (1987)) and human glucocerebrosidase (Martin et al., DNA, 7: 99 (1988)), can also be used to provide for secretion in insects.

Suitable yeast signal sequences can be derived from genes for secreted yeast proteins, such as the yeast invertase gene (EPO Publ. No. 012 873; JPO Publ. No. 62,096,086) and the A-factor gene (U.S. Pat. No. 4,588,684). Alternatively, sequences of non-yeast origin, such as from interferon, exist that also provide for secretion in yeast (EPO Publ. No. 060 057).

The term “Solubility” refers to the amount of a substance that can be dissolved in, a unit volume of solvent For example, solubility as used herein refers to the ability of a tandem polypeptide to be resuspended in a volume of solvent, such as a biological buffer.

A “Suppressible stop codon” is a codon that serves as a stop codon to translation of an RNA that contains the suppressible stop codon when the RNA is translated in a cell that is not a suppressing cell. However, when the RNA is translated in a cell that is a suppressing cell, the suppressing cell will produce a transfer RNA that recognizes the suppressible stop codon and provides for insertion of an amino acid into the growing polypeptide chain. This action allows translation of the RNA to continue past the suppressible stop codon. Suppressible stop codons are sometimes referred to as nonsense mutations. Suppressible stop codons are well known in the art and include such examples as amber mutations (UAG) and ochre mutations (UAA). Numerous suppressing cells exist which insert an amino acid into a growing polypeptide chain at a position corresponding to a suppressible stop codon. Examples of suppressors, the codon recognized, and the inserted amino acid include: supD, amber, serine; supE, amber, glutamine; supF, amber, tyrosine; supB, amber and ochre, glutamine; and supC, amber and ochre, tyrosine. Other suppressors are known in the art. Additionally, numerous cells are known in the art that are suppressing cells. Examples of such cells include, but are not limited to, the bacterial strains: 71/18 (supE); BB4 (supF58 and supE44); BNN102 (supE44); C600 (supE44); and CSH18 (supE). Those of skill in the art realize that many suppressing cells are known and are obtainable from ATCC or other commercial sources. A suppressible stop codon can be used to insert a specific amino acid into a polypeptide chain at a specific location. Such insertion can be used to create a specific amino acid sequence in a polypeptide that serves as a cleavage site for a cleavage agent. Through selection of an appropriate suppressible stop codon and translation of an RNA containing the suppressible stop codon in an appropriate cell, one skilled in the art can control what cleavage agent can cleave a polypeptide chain at a given position.

A “Tandem polypeptide” as defined herein is a protein having an inclusion body fusion partner operably linked to a preselected polypeptide that may optionally include additional amino acids. A tandem polypeptide is further defined as forming an inclusion body when expressed in a cell.

A “Tissue specific protease” refers to a proteolytic enzyme that is expressed in specific cells at a higher level than in other cells of a different type. Prostate specific antigen is an example of a tissue specific protease.

A “Transcription terminator sequence” is a signal within DNA that functions to stop RNA synthesis at a specific point along the DNA template. A transcription terminator may be either rho factor dependent or independent. An example of a transcription terminator sequence is the T7 terminator. Transcription terminators are known in the art and may be isolated from commercially available vectors according to recombinant methods known in the art. (Sambrook and Russell, Molecular Cloning: A Laboratory Manual, 3rd edition (Jan. 15, 2001) Cold Spring Harbor Laboratory Press, ISBN: 0879695765; Stratagene, La Jolla, Calif.).

“Transformation” refers to the insertion of an exogenous nucleic acid sequence into a host cell, irrespective of the method used for the insertion. For example, direct uptake, transduction, f-mating or electroporation may be used to introduce a nucleic acid sequence into a host cell. The exogenous nucleic acid sequence may be maintained as a non-integrated vector, for example, a plasmid, or alternatively, may be integrated into the host genome.

A “Translation initiation sequence” refers to a DNA sequence that codes for a sequence in a transcribed mRNA that is optimized for high level translation initiation. Numerous translation initiation sequences are known in the art. These sequences are sometimes referred to as leader sequences. A translation initiation sequence may include an optimized ribosome binding site. In the present invention, bacterial translational start sequences are preferred. Such translation initiation sequences are well known in the art and may be obtained from bacteriophage T7, bacteriophage φ10, and the gene encoding ompT. Those of skill in the art can readily obtain and clone translation initiation sequences from a variety of commercially available plasmids, such as the pET (plasmid for expression of T7 RNA polymerase) series of plasmids. (Stratagene, La Jolla, Calif.).

A “variant” polypeptide is a polypeptide derived from the native polypeptide by deletion or addition of one or more amino acids to the N-terminal and/or C-terminal end of the native polypeptide; deletion or addition of one or more amino acids at one or more sites in the native protein; or substitution of one or more amino acids at one or more sites in the native protein. Such substitutions or insertions are preferably conservative amino acid substitutions. Methods for such manipulations are generally known in the art. (Kunkel, Proc. Natl. Acad. Sci. USA, 82:488, (1985); Kunkel et al., Methods in Enzymol., 154:367 (1987); U.S. Pat. No. 4,873,192; Walker and Gaastra, eds. (1983) Techniques in Molecular Biology (MacMillan Publishing Company, New York)) and the references cited therein. Also, kits are commercially available for mutating DNA. (Quick change Kit, Stratagene, La Jolla, Calif.). Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.).

A “Vector” includes, but is not limited to, any plasmid, cosmid, bacteriophage, yeast artificial chromosome, bacterial artificial chromosome, f-factor, phagemid or virus in double or single stranded linear or circular form which may or may not be self transmissible or mobilizable, and which can transform a prokaryotic or eukaryotic host either by integration into the cellular genome or exist extrachromosomally (e.g. autonomous replicating plasmid with an origin of replication).

Specifically included are shuttle vectors by which are DNA vehicles capable, naturally or by design, of replication in two different host organisms (e.g. bacterial, mammalian, yeast or insect cells).

### BRIEF DESCRIPTION OF THE DRAWINGS

FIG. 1 is the pBN121 plasmid map. Ori=the origin of replication from pMB1; Kan^{R}=kanamycin resistance gene; Tac=tac promoter; LacIq=lac repressor gene; GST=terminator.

FIG. 2 shows a hydrophobicity plot of an inclusion body fusion partner (SEQ ID NO:1).

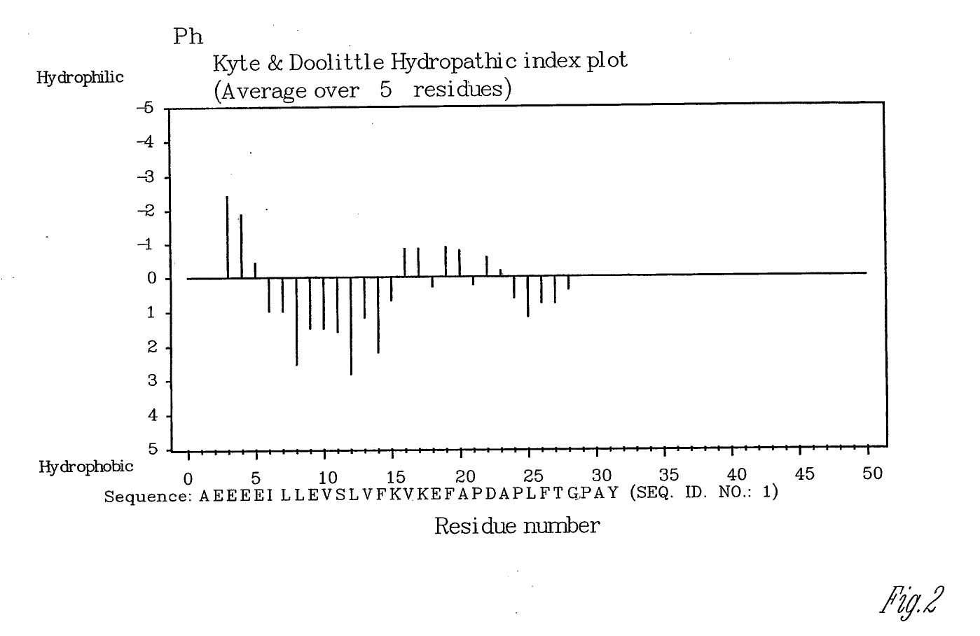

FIG. 3 shows the amino acid and nucleic acid sequences (SEQ ID NOs 78 and 79, respectively) for the expression cassette of pBN121-T7tagPh-CH-GRF(1-44)CH.

FIG. 4 shows the amino acid and nucleic acid sequences (SEQ ID NOs 80 and 81, respectively)for the expression cassette of pBN121-T7tagPh-CPGM-GLP-1(7-36)CHPG.

FIG. 5 shows the amino acid and nucleic acid sequences (SEQ ID NOs 82 and 83, respectively)for the expression cassette of pBN121-T7tagPh-GGGR-GLP-1(7-36)AFA.

FIG. 6 is the pBN122-M-GLP-1(7-36)AFAFGGGPG-T7tagPh plasmid map. Ori=the origin of replication from pMB1; Kan^{R}=kanamycin resistance gene; Tac=tac promoter; LacIq=lac repressor gene; GST=terminator.

FIG. 7 shows the amino acid and nucleic acid sequences (SEQ ID NOs 84 and 85, respectively)for the expression cassette of pBN122-M-GLP-1(7-36)AFAFGGGPG-T7tagPh.

FIG. 8 shows the amino acid and nucleic acid sequences (SEQ ID NOs 86 and 87, respectively)for the expression cassette of pBN121-T7tagPh-VDDR-GLP-2(1-33)A2G.

FIG. 9 is the SDS-PAGE analysis of lysates obtained from cells that contain a nucleic acid construct of the invention. Cells were lysed by sonication in 300 μl 10 mM Tris, 1 mM EDTA (pH 8) buffer and centrifuged for 5 minutes to separate the supernatants and inclusion bodies. The inclusion bodies were resuspended in 300 μl water and mixed with 2× sample buffer. After heating at 85° C. for 10 minutes, 20 μl of each sample was applied to the gel. Lane 1: Invitrogen Multi Mark. Lanes 2 and 3: inclusion bodies from induced HMS174 cells containing pBN121-T7tagPh-CPGM-GLP-1(7-36)CHPG. Lanes 4 and 5: inclusion bodies form induced BL21 cells containing pBN121-T7tagPh-CPGM-GLP-1(7-36)CHPG. Lanes 6 and 7: inclusion bodies from induced HMS174 cells with pBN121-T7tagPh-CH-GRF(1-44)CH. Lanes 8 and 9: inclusion bodies from induced BL21 cells containing pBN121-T7tagPh-CH-GRF(1-44)CH.

FIG. 10 shows the amino acid, and nucleic acid sequences (SEQ ID NOs 88 and 89, respectively) for the expression cassette of pBN121-M-PTH(1-84).

FIG. 11 shows the amino acid and nucleic acid sequences (SEQ ID NOs 90 and 91, respectively) for the expression cassette pBN121-T7tag-CH-PTH (1-84).

FIG. 12 shows the amino acid and nucleic acid sequences (SEQ ID NOs 92 and 93, respectively) for the expression cassette of pBN121-T7tagPh-CH-PTH (1-84).

FIG. 13 illustrates an SDS-PAGE analysis. Lane 1: lysate from induced BL21 cells containing pBN121-M-PTH(1-84). Lane 2: lysate from induced BL21 cells containing pBN121-T7tag-CH-PTH(1-84). Lane 3: lysate from induced BL21 cells containing pBN121-T7tagPh-CH-PTH(1-84).

### DETAILED DESCRIPTION OF THE INVENTION

The invention provides methods and materials that allow a preselected polypeptide to be efficiently expressed in a cell. A preselected polypeptide is inserted into an expression cassette provided by the invention. The expression cassette causes a preselected polypeptide to be operably linked to an inclusion body fusion partner to form a tandem polypeptide. The tandem polypeptide will form an inclusion body in the cell in which the tandem polypeptide is expressed.

A significant advantage of producing polypeptides by recombinant DNA techniques rather than by isolating and purifying a polypeptide from a natural source is that equivalent quantities of the protein can be produced by using less starting material than would be required for isolating the polypeptide from a natural source. Furthermore, inclusion body formation allows a tandem polypeptide to be more readily purified and is thought to protect the tandem polypeptide against unwanted degredation within the cell. Producing the polypeptide through use of recombinant techniques also permits the protein to be isolated in the absence of some molecules normally present in native cells. For example, polypeptide compositions free of human polypeptide contaminants can be produced because the only human polypeptide produced by the recombinant non-human host is the recombinant polypeptide at issue. Furthermore, potential viral agents from natural sources and viral components pathogenic to humans are also avoided.

I. Expression Cassette

The invention provides an expression cassette capable of directing the expression of a tandem polypeptide which includes a preselected polypeptide that is operably linked to an inclusion body fusion partner. The invention also provides an expression cassette capable of directing the expression of a tandem polypeptide which includes a preselected polypeptide that is operably linked to an inclusion body fusion partner and a cleavable peptide linker. The invention also provides an expression cassette capable of directing the expression of a tandem polypeptide which includes a preselected polypeptide that is operably linked to an inclusion body fusion partner and a fusion tag. The invention also provides an expression cassette capable of directing the expression of a tandem polypeptide which includes a preselected polypeptide that is operably linked to an inclusion body fusion partner, a cleavable linker peptide, and a fusion tag. The invention also provides an expression cassette capable of directing the expression of a tandem polypeptide which includes a preselected polypeptide that is operably linked to an inclusion body fusion partner, and independently operably linked to one or more cleavable peptide linkers, or to one or more fusion tags in any order that will cause a tandem polypeptide to form an inclusion body.

Promoters

The expression cassette of the invention includes a promoter. Any promoter able to direct transcription of the expression cassette may be used. Accordingly, many promoters may be included within the expression cassette of the invention. Some useful promoters include, constitutive promoters, inducible promoters, regulated promoters, cell specific promoters, viral promoters, and synthetic promoters. A promoter is a nucleotide sequence which controls expression of an operably linked nucleic acid sequence by providing a recognition site for RNA polymerase, and possibly other factors, required for proper transcription. A promoter includes a minimal promoter, consisting only of all basal elements needed for transctiption initiation, such as a TATA-box and/or other sequences that serve to specify the site of transcription initiation. A promoter may be obtained from a variety of different sources. For example, a promoter may be derived entirely from a native gene, be composed of different elements derived from different promoters found in nature, or be composed of nucleic acid sequences that are entirely synthetic. A promoter may be derived from many different types of organisms and tailored for use within a given cell.

Promoters for Use in Bacterial Cells

For expression of a tandem polypeptide in a bacterium, an expression cassette having a bacterial promoter will be used. A bacterial promoter is any DNA sequence capable of binding bacterial RNA polymerase and initiating the downstream (3″) transcription of a coding sequence into mRNA. A promoter will have a transcription initiation region which is usually placed proximal to the 5′ end of the coding sequence. This transcription initiation region usually includes an RNA polymerase binding site and a transcription initiation site. A second domain called an operator may be present and overlap an adjacent RNA polymerase binding site at which RNA synthesis begins. The operator permits negatively regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in E. coli (Raibaud et al., Ann. Rev. Genet., 18:173 (1984)). Regulated expression may therefore be positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) (Chang et al., Nature, 198:1056 (1977)), and maltose. Additional examples include promoter sequences derived from biosynthetic enzymes such as tryptophan (trp) (Goeddel et al., N.A.R., 8: 4057 (1980); Yelverton et al., N.A.R., 9: 731 (1981); U.S. Pat. No. 4,738,921; and EPO Publ. Nos. 036 776 and 121 775). The β-lactamase (bla) promoter system (Weissmann, “The cloning of interferon and other mistakes”, in: Interferon 3 (ed. I. Gresser), 1981), and bacteriophage lambda P_{L} (Shimatake et al., Nature, 292:128 (1981)) and T5 (U.S. Pat. No. 4,689,406) promoter systems also provide useful promoter sequences. A preferred promoter is the Chlorella virus promoter (U.S. Pat. No.6,316,224).

Synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter (U.S. Pat. No. 4,551,433). For example, the tac promoter is a hybrid trp-lac promoter comprised of both trp promoter and lac operon sequences that is regulated by the lac repressor (Amann et al., Gene 25:167 (1983); de Boer et al., Proc. Natl. Acad. Sci. USA, 80: 21 (1983)). Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system (Studier et al., J. Mol. Biol., 189: 113 (1986); Tabor et al., Proc. Natl. Acad. Sci. USA, 82:1074 (1985)). In addition, a hybrid promoter can also be comprised of a bacteriophage promoter and an E. coli operator region (EPO Publ. No. 267 851).

Promoters for Use in Insect Cells

An expression cassette having a baculovirus promoter can be used for expression of a tandem polypeptide in an insect cell. A baculovirus promoter is any DNA sequence capable of binding a baculovirus RNA polymerase and initiating transcription of a coding sequence into mRNA. A promoter will have a transcription initiation region which is usually placed proximal to the 5′ end of the coding sequence. This transcription initiation region usually includes an RNA polymerase binding site and a transcription initiation site. A second domain called an enhancer may be present and is usually distal to the structural gene. A baculovirus promoter may be a regulated promoter or a constitutive promoter. Useful promoter sequences may be obtained from structural genes that are transcribed at times late in a viral infection cycle. Examples include sequences derived from the gene encoding the baculoviral polyhedron protein (Friesen et al., “The Regulation of Baculovirus Gene Expression”, in: The Molecular Biology of Baculoviruses (ed. Walter Doerfler), 1986; and EPO Publ. Nos. 127 839 and 155 476) and the gene encoding the baculoviral p10 protein (Vlak et al., J. Gen. Virol. 69: 765 (1988)).

Promoters for Use in Yeast Cells

Promoters that are functional in yeast are known to those of ordinary skill in the art. In addition to an RNA polymerase binding site and a transcription initiation site, a yeast promoter may also have a second region called an upstream activator sequence. The upstream activator sequence permits regulated expression that may be induced. Constitutive expression occurs in the absence of an upstream activator sequence. Regulated expression may be either positive or negative, thereby either enhancing or reducing transcription.

Promoters for use in yeast may be obtained from yeast genes that encode enzymes active in metabolic pathways. Examples of such genes include alcohol dehydrogenase (ADH) (EPO Publ. No. 284 044), enolase, glucokinase, glucose-6-phosphate isomerase, glyceraldehyde-3-phosphatedehydrogenase (GAP or GAPDH), hexokinase, phosphofructokinase, 3-phosphoglyceratemutase, and pyruvate kinase (PyK). (EPO Publ. No. 329 203). The yeast PHOS gene, encoding acid phosphatase, also provides useful promoter sequences. (Myanohara et al., Proc. Nati. Acad. Sci. USA. 80: 1 (1983)).

Synthetic promoters which do not occur in nature may also be used for expression in yeast. For example, upstream activator sequences from one yeast promoter may be joined with the transcription activation region of another yeast promoter, creating a synthetic hybrid promoter. Examples of such hybrid promoters include the ADH regulatory sequence linked to the GAP transcription activation region (U.S. Pat. Nos. 4,876,197 and 4,880,734). Other examples of hybrid promoters include promoters which consist of the regulatory sequences of either the ADH2, GAL4, GAL10, or PHO5 genes, combined with the transcriptional activation region of a glycolytic enzyme gene such as GAP or PyK (EPO Publ. No. 164 556). Furthermore, a yeast promoter can include naturally occurring promoters of non-yeast origin that have the ability to bind yeast RNA polymerase and initiate transcription. Examples of such promoters are known in the art. (Cohen et al., Proc. Natl. Acad. Sci. USA, 77: 1078 (1980); Henikoff et al., Nature 283:835 (1981); Hollenberg et al., Curr. Topics Microbiol. Immunol., 96: 119 (1981); Hollenberg et al., “The Expression of Bacterial Antibiotic Resistance Genes in the Yeast Saccharomyces cerevisiae”, in: Plasmids of Medical, Environmental and Commercial Importance (eds. K. N. Timmis and A. Puhler), 1979; Mercerau-Puigalon et al., Gene, 11:163 (1980); Panthier et al., Curr. Genet. 2:109 (1980)).

Promoters for Use in Mammalian Cells

Many mammalian promoters are known in the art that may be used in conjunction with the expression cassette of the invention. Mammalian promoters often have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter may also contain an upstream promoter element, usually located within 100 to 200 bp upstream of the TATA box. An upstream promoter element determines the rate at which transcription is initiated and can act in either orientation (Sambrook et al., “Expression of Cloned Genes in Mammalian Cells”, in: Molecular Cloning: A Laboratory Manual, 2nd ed., 1989).

Mammalian viral genes are often highly expressed and have a broad host range; therefore sequences encoding mammalian viral genes often provide useful promoter sequences. Examples include the SV40 early promoter, mouse mammary tumour virus LTR promoter, adenovirus major late promoter (Ad MLP), and herpes simplex virus promoter. In addition, sequences derived from non-viral genes, such as the murine metallothioneih gene, also provide useful promoter sequences. Expression may be either constitutive or regulated.

A mammalian promoter may also be associated with an enhancer. The presence of an enhancer will usually increase transcription from an associated promoter. An enhancer, is a regulatory DNA sequence that can stimulate transcription up to 1000-fold when linked to homologous or heterologous promoters, with synthesis beginning at the normal RNA start site. Enhancers are active when they are placed upstream or downstream from the transcription initiation site, in either normal or flipped orientation, or at a distance of more than 1000 nucleotides from the promoter. (Maniatis et al., Science, 236:1237 (1987); Alberts et al., Molecular Biology of the Cell, 2nd ed., 1989)). Enhancer elements derived from viruses are often times useful, because they usually have a broad host range. Examples include the SV40 early gene enhancer (Dijkema et al., EMBO J., 4:761 (1985) and the enhancer/promoters derived from the long terminal repeat (LTR) of the Rous Sarcoma Virus (Gorman et al., Proc. Natl. Acad. Sci. USA, 79:6777 (1982b)) and from human cytomegalovirus (Boshart et al., Cell, 41: 521 (1985)). Additionally, some enhancers are regulatable and become active only in the presence of an inducer, such as a hormone or metal ion (Sassone-Corsi and Borelli, Trends Genet., 2:215 (1986); Maniatis et al., Science, 236:1237 (1987)).

It is understood that many promoters and associated regulatory elements may be used within the expression cassette of the invention to transcribe an encoded tandem polypeptide. The promoters described above are provided merely as examples and are not to be considered as a complete list of promoters that are included within the scope of the invention.

Translation Initiation Sequence

The expression cassette of the invention may contain a nucleic acid sequence for increasing the translation efficiency of an mRNA encoding a tandem polypeptide of the invention. Such increased translation serves to increase production of the tandem polypeptide. The presence of an efficient ribosome binding site is useful for gene expression in prokaryotes. The bacterial mRNA a conserved stretch of six nucleotides, the Shine-Dalgamo sequence, is usually found upstream of the initiating AUG codon. (Shine et al., Nature, 254: 34 (1975)). This sequence is thought to promote ribosome binding to the mRNA by base pairing between the ribosome binding site and the 3′ end of Escherichia coli 16S rRNA. (Steitz et al., “Genetic signals and nucleotide sequences in messenger RNA”, in: Biological Regulation and Development: Gene Expression (ed. R. F. Goldberger), 1979)). Such a ribosome binding site, or operable derivatives thereof, are included within the expression cassette of the invention.

A translation initiation sequence can be derived from any expressed Escherichia coli gene and can be used within an expression cassette of the invention. Preferably the gene is a highly expressed gene. A translation initiation sequence can be obtained via standard recombinant methods, synthetic techniques, purification techniques, or combinations thereof, which are all well known. (Ausubel et al., Current Protocols in Molecular Biology Green Publishing Associates and Wiley Interscience, NY. (1989); Beaucage and Caruthers, Tetra. Letts., 22:1859 (1981); VanDevanter et al., Nucleic Acids Res., 12:6159 (1984). Alternatively, translational start sequences can be obtained from numerous commercial vendors. (Operon Technologies; Life Technologies Inc, Gaithersburg, Md.). In a preferred embodiment, the T7 leader sequence is used. The T7tag leader sequence is derived from the highly expressed T7 Gene 10 cistron. Other examples of translation initiation sequences include, but are not limited to, the maltose-binding protein (Mal E gene) start sequence (Guan et al., Gene 67:21 (1997)) present in the pMalc2 expression vector (New England Biolabs, Beverly, Mass.) and the translation initiation sequence for the following genes: thioredoxin gene (Novagen, Madison, Wis.), Glutathione-S-transferase gene (Pharmacia, Piscataway, N.J.), β-galactosidase gene, chloramphenicol acetyltransferase gene and E. coli Trp E gene (Ausubel et al., 1989, Current Protocols in Molecular Biology, Chapter 16, Green Publishing Associates and Wiley Interscience, NY).

Eucaryotic mRNA does not contain a Shine-Dalgarno sequence. Instead, the selection of the translational start codon is usually determined by its proximity to the cap at the 5′ end of an mRNA. The nucleotides immediately surrounding the start codon in eucaryotic mRNA influence the efficiency of translation. Accordingly, one skilled in the art can determine what nucleic acid sequences will increase translation of a tandem polypeptide encoded by the expression cassette of the invention. Such nucleic acid sequences are within the scope of the invention.

Cleavable Peptide Linker

A cleavable peptide linker is an amino acid sequence that can be recognized by a cleavage agent and cleaved. Many amino acid sequences are known that are recognized and cleaved. Examples of cleavage agents and their recognition sites include, but are not limited to, chymotrypsin cleaves after phenylalanine, threonine, or tyrosine; thrombin cleaves after arginine, trypsin cleaves after lysine or arginine, and cyanogen bromide cleaves after methionine. Those of skill in the art realize that many amino acid sequences exist that may be used as a cleavable peptide linker within the scope of the invention. The expression cassette of the invention may encode a tandem polypeptide containing an inclusion body fusion partner operably linked to a preselected polypeptide and a cleavable peptide linker. Thus, an expression cassette of the invention can be designed to encode a tandem polypeptide containing a cleavable peptide linker that can be cleaved by a specific agent. In addition, the expression cassette of the invention may be designed to encode a tandem polypeptide containing multiple cleavable peptide linkers. These cleavable peptide linkers may be cleaved by the same cleavage agent or by different cleavage agents. The cleavable peptide linkers may also be positioned at different positions within the tandem polypeptide. Such a tandem polypeptide may be treated with select cleavage agents at different times to produce different cleavage products of the tandem polypeptide.

Furthermore, an expression cassette of the invention maybe designed to express a tandem polypeptide containing a tissue specific protease that will promote cleavage of the tandem polypeptide in a tissue specific manner. For example, prostate specific antigen is a serine protease expressed in cells lining prostatic ducts. Prostate specific antigen exhibits a preference for cleavage at the amino acid sequence serine-serine-(tyrosine/phenylalanine)-tyrosine↓serine-(glycine/serine). (Coombs et al., Chem. Biol., 5:475 (1998)). Accordingly, a tandem polypeptide can be designed that is specifically cleaved in prostate tissue. Thus, the expression cassette of the invention may be used to express a tandem polypeptide that is a prodrug. Such a prodrug can be activated at a specific tissue in the body of a patient in need thereof. Such a tandem polypeptide offers the advantage that the prodrug is only activated at the site of action and potentially toxic effects on other tissues can be avoided. Those of skill in the art will recognize that the expression cassette of the invention can be used to express many different tandem polypeptides that contain a cleavable peptide linker that is tissue specific.

Inclusion Body Fusion Partner

The expression cassette of the present invention encodes an inclusion body fusion partner that is operably linked to a preselected polypeptide. It has been surprisingly found that the amino acid sequence of an inclusion body fusion partner can be altered to produce inclusion bodies that exhibit useful characteristics. These useful characteristics provide isolation enhancement to inclusion bodies that are formed from tandem polypeptides that include an inclusion body fusion partner of the invention. Isolation enhancement allows a tandem polypeptide containing an inclusion body fusion partner that is fused to a preselected polypeptide to be isolated and purified more readily than the preselected polypeptide in the absence of the inclusion body fusion partner. For example, the inclusion body fusion partner may be altered to produce inclusion bodies that are more or less soluble under a certain set of conditions. Those of skill in the art realize that solubility is dependent on a number of variables that include, but are not limited to, pH, temperature, salt concentration, and protein concentration. Thus, an inclusion body fusion partner of the invention may be altered to produce an inclusion body having desired solubility under differing conditions.

In another example, an inclusion body fusion partner of the invention may be altered to produce inclusion bodies that contain tandem polypeptides having greater or lesser self-association. Self-association refers to the strength of the interaction between two or more tandem polypeptides that form an inclusion body and that contain an inclusion body fusion partner of the invention. Such self-association may be determined though use of a variety of known methods used to measure protein-protein interactions. Such methods are known in the art and have been described. Freifelder, Physical Biochemistry: Applications to Biochemistry and Molecular Biology, W.H. Freeman and Co., 2nd edition, New York, N.Y. (1982). Self-adhesion can be used to produce inclusion bodies that exhibit varying stability to purification. For example, greater self-adhesion may be desirable to stabilize inclusion bodies against dissociation in instances where harsh conditions are used to isolate the inclusion bodies from a cell. Such conditions may be encountered if inclusion bodies are being isolated from cells having thick cell walls. However, where mild conditions are used to isolate the inclusion bodies, less self-adhesion may be desirable as it may allow the tandem polypeptides composing the inclusion body to be more readily solubilized or processed. Accordingly, an inclusion body fusion partner of the invention may be altered to provide a desired level of self-adhesion for a given set of conditions.

Such an inclusion body fusion partner may be linked to the amino-terminus, the carboxyl-terminus, or both termini of a preselected polypeptide to form a tandem polypeptide. An inclusion body fusion partner is of an adequate size to cause an operably linked preselected polypeptide to form an inclusion body. It is preferred that the inclusion body fusion partner is 100 or less amino acids, more preferably 50 or less amino acids, and most preferably 31 or less amino acids in length.

In one example, the inclusion body fusion partner has an amino acid sequence corresponding to: AEEEEILLEVSLVFKVKEFAPDAPLFTGPAY (SEQ ID NO: 1). This amino acid sequence corresponds to a carboxyl-terminal portion of the Hyphantria cunea nucleopolyhedrovirus polyhedrin gene which has been surprisingly found to be alterable in order to produce tandem polypeptides which form inclusion bodies that exhibit isolation enhancement. The inclusion body fusion partner can also have an amino acid sequence that is a variant of SEQ ID NO: 1 and which causes inclusion body formation by an operably linked preselected polypeptide. An inclusion body fusion partner can also have an amino acid sequence corresponding to SEQ ID NO: 1, or a variant thereof, in addition to other amino acids which cause inclusion body formation by an operably linked preselected polypeptide. An example of prefered additional amino acids to which the inclusion body fusion partner can be linked is the T7 tag sequence: MASMTGGQQMGRGS (SEQ ID NO: 2).

An inclusion body fusion partner of the invention can be identified by operably linking an inclusion body fusion partner to a preselected polypeptide and determining if the tandem polypeptide produced forms an inclusion body within a cell. Recombinant methods which may be used to construct such variant inclusion body fusion partners are well known in the art and have been reported. Sambrook and Russell, Molecular Cloning: A Laboratory Manual, 3rd edition (Jan. 15, 2001) Cold Spring Harbor Laboratory Press, ISBN: 0879695765.

An inclusion body fusion partner variant also can be identified by comparing its sequence homology to SEQ ID NO: 1. A protein fragment possessing 75% or more amino acid sequence homology, especially 85-95%, to SEQ ID NO: 1 is considered a variant and is encompassed by the present invention.

Mathematical algorithms, for example the Smith-Waterman algorithm, can also be used to determine sequence homology. (Smith & Waterman, J. Mol. Biol., 147:195 (1981); Pearson, Genomics, 11:635 (1991)). Although any sequence algorithm can be used to identify a variant, the present invention defines a variant with reference to the Smith-Waterman algorithm, where SEQ ID NO: 1 is used as the reference sequence to define the percentage of homology of peptide homologues over its length. The choice of parameter values for matches, mismatches, and inserts or deletions is arbitrary, although some parameter values have been found to yield more biologically realistic results than others. One preferred set of parameter values for the Smith-Waterman algorithm is set forth in the “maximum similarity segments” approach, which uses values of 1 for a matched residue and −⅓ for a mismatched residue (a residue being either a single nucleotide or single amino acid) (Waterman, Bulletin of Mathematical Biology 46:473 (1984)). Insertions and deletions x, are weighted as xₖ=1+k/3, where k is the number of residues in a given insert or deletion. Preferred variant inclusion body fusion partners are those having greater than 75% amino acid sequence homology to SEQ ID NO: 1 using the Smith-Waterman algorithm. More preferred variants have greater than 90% amino acid sequence homology. Even more preferred variants have greater than 95% amino acid sequence homology, and most preferred variants have at least 98% amino acid sequence homology.

Open Reading Frames

Numerous nucleic acid sequences can be inserted into an expression cassette or a nucleic acid construct of the invention and used to produce many different preselected polypeptides. Such preselected polypeptides include those that are soluble or insoluble within the cell in which they are expressed. One skilled in the art can determine if a nucleic acid sequence can be expressed using the expression cassette of the invention by inserting the nucleic acid sequence into an expression cassette and determining if a corresponding polypeptide is produced when the nucleic acid construct is inserted into an appropriate cell.

More than one copy of an open reading frame can be inserted into an expression cassette of the invention. Preferably, a cleavable peptide linker is inserted between open reading frames if more than one is inserted into an expression cassette of the invention. Such a construct allows the tandem polypeptide to be cleaved by a cleavage agent to produce individual preselected polypeptides from the polyprotein expressed from an expression cassette containing more than one open reading frame.

An expression cassette or nucleic acid construct of the invention is thought to be particularly advantageous for producing preselected polypeptides that are degraded within a cell in which they are expressed. Short polypeptides are examples of such preselected polypeptides. The present expression cassettes and nucleic acid constructs are also thought to be advantageous for producing preselected polypeptides that are difficult to purify from cells. For example, operably linking an inclusion body fusion partner to a preselected polypeptide that would normally associate tightly with a cell wall or membrane may allow the protein to be more easily purified from an inclusion body.

Preferred open reading frames encode glucagon-like peptide-1 (GLP-1), glucagon-like peptide-2 (GLP-2), parathyroid hormone (PTH), and growth hormone releasing factor (GRF). Other preferred open reading frames include those that encode glucagon-like peptides, analogs of glucagon-like peptide-1, analogs of glucagon-like peptide-2, GLP (7-36), and analogs of growth hormone releasing factor. Such analogs may be identified by their ability to bind to their respective receptors. For example, an analog of glucagon-like peptide-1 will detectably bind to glucagon-like protein-1 receptor.

One skilled in the art realizes that many open reading frames may be used within an expression cassette or nucleic acid construct of the invention. Examples of suitable open reading frames and their corresponding polypeptides include, but are not limited to, those listed in Tables II and III.

Suppressable Stop Codon

The expression cassette of the invention may also include a suppressible stop codon. A suppressible stop codon is sometimes refered to as a nonsense mutation. A suppressible stop codon serves as a signal to end translation of an RNA at the location of the suppressible stop codon in the absence of a suppressor. However, in the presence of a suppressor, translation will continue through the suppressible stop codon until another stop codon signals the end of translation of the RNA. Suppressible stop codons and suppressors are known in the art. Sambrook and Russell, Molecular Cloning: A Laboratory Manual, 3rd edition (Jan. 15, 2001) Cold Spring Harbor Laboratory Press, ISBN: 0879695765. Such codons are exemplified by ochre (UAA) and amber (UAG) codons. Suppressible stop codons can be suppressed in cells that encode a tRNA that recognizes the codon and facilitates insertion of an amino acid into the polypeptide chain being translated from the RNA containing the codon. Different cells contain different tRNAs that facilitate insertion of different amino acids into the polypeptide chain at the suppressible stop codon. For example, an amber codon can be suppressed by supD, supE, supF, supB and supC bacterial strains which insert serine, glutamine, tyrosine, glutamine, and, tyrosine respectively into a polypeptide. An ochre codon can be suppressed by supB and supC bacterial strains which insert glutamine and tyrosine respectively into a polypeptide chain. Additional suppressible codons and suppressors maybe used within the expression cassette of the invention.

Use of a suppressible stop codon in the expression cassette of the invention allows for the production of polypeptides that have a different amino acid inserted at the position coded for by the suppressible stop codon without altering the expression cassette. The use of a suppressible stop codon also allows tandem polypeptides of differing molecular weights to be expressed from the same expression cassette. For example, an expression cassette designed to contain an amber mutation can be expressed in a non-suppressing strain to produce a tandem polypeptide that terminates at the amber codon. The same expression cassette can be expressed in a supE Escherichia coli to produce a tandem polypeptide having a glutamine inserted into the fusion polypeptide at the amber mutation. This tandem polypeptide may also include an addition amino acid sequence, such as a fusion tag that is terminated with a second stop codon. An expression cassette of the invention that contains a suppressible stop codon provides for the production of numerous variations of a tandem polypeptide that can be expressed from the same expression cassette. Such tandem polypeptide variations will depend on the combination of the suppressible stop codon used within the expression cassette and the cell in which the expression cassette is inserted.

One or more cleavage agent recognition sites may be introduced into a tandem polypeptide expressed from an expression cassette of the invention through use of an appropriate suppressible stop codon and suppressing cell. For example, a tandem polypeptide can be designed to contain a chymotrypsin cleavage site through use of an expression cassette that encodes the tandem polypeptide and has an amber codon in a supF or supC bacterium such that a tyrosine is inserted into the fusion polypeptide. In another example, a Neisseria type 2 IgA protease recognition site can be created through use of an amber containing expression cassette in a supD cell. In yet another example, a recognition site for Plum pox potyvirus Nia protease, Poliovirus 2Apro protease, or Nia Protease (tobacco etch virus) can be created through appropriate use of an expression cassette containing an amber or ochre codon in a supF or a supC cell. Accordingly, an expression cassette of the invention may contain more than one supressible codon to express a tandem polypeptide that can contain more than one engineered cleavage agent recognition site.

Furthermore, an expression cassette of the invention may be used to express a tandem polypeptide having a preselected amino acid inserted at any position along the polypeptide chain that corresponds to a suppressible stop codon. Briefly, an aminoacyl-tRNA synthetase may be introduced into a cell which specifically acylates a suppressor tRNA with a predetermined amino acid. An expression expression cassette containing a suppressible stop codon which may be suppressed by the acylated-tRNA can be expressed in the cell. This will cause a tandem polypeptide to be produced that has the predetermined amino acid inserted into the tandem polypeptide at a position corresponding to the suppressible stop codon. Such a system allows for the design and production of a tandem polypeptide having one or more cleavage agent recognition sites. This in turn allows for the production of tandem polypeptides that can be cleaved by tissue specific proteases. Methods to facilitate the insertion of a specific amino acid into polypeptide chain are known in the art and have been reported. Kowal et al., Proc. Natl. Acad. Sci. (USA) 98:2268 (2001).

An expression cassette of the invention may also be used to produce tandem polypeptides having an amino acid analog inserted at any amino acid position. Briefly, a tRNA that is able to suppress a suppressible stop codon is aminoacylated with a desired amino acid analog in vitro according to methods known in the art. The aminoacylated suppressor tRNA can then be imported into a cell containing an expression cassette of the invention. The imported tRNA then facilitates incorporation of the amino acid analog at a position of the tandem polypeptide expressed from the expression cassette at a position corresponding to that of the suppressible stop codon. Such methods are thought to be particularly useful in mammalian cells, such as COS1 cells. Kohrer et al., Proc. Natl. Acad. Sci. USA, 98:14310 (2001).

Fusion Tag

An expression cassette of the invention can optionally express a tandem polypeptide containing a fusion tag. A fusion tag is an amino acid seqeunce that confers a useful property to the tandem polypeptide. In one example, a fusion tag may be a ligand binding domain that can be used to purify the tandem polypeptide by applying a tandem polypeptide containing the fusion tag to separation media containing the ligand. Such a combination is exemplified by application of a tandem polypeptide containing a glutathione-S-transferase domain to a chromatographic column containing glutathione-linked separation media. In another example, a tandem polypeptide containing a polyhistidine fusion tag may be applied to a nickel column for purification of the tandem polypeptide. In yet another example, a fusion tag can be a ligand. Such a tandem polypeptide can include glutathione as a fusion tag and be applied to a chromatographic column containing glutathione-S-transferase-linked separation media. In still another example, the fusion tag may be an antibody epitope. Such a combination is exemplified by a tandem polypeptide containing maltose binding protein as a fusion tag. Such a tandem polypeptide can be applied to separation media containing an anti-maltose binding protein. Such systems are known in the art and are commercially available. (New England Biolabs, Beverly, Mass.; Stratagene, La Jolla, Calif.). Those of skill in the art realize that numerous fusion tags may incorporated into the expression cassette of the invention.

Termination Sequences

Termination Sequences for Use in Bacteria

Usually, transcription termination sequences recognized by bacteria are regulatory regions located 3′ to the translation stop codon, and thus together with the promoter flank the coding sequence. These sequences direct the transcription of an mRNA which can be translated into the polypeptide encoded by the DNA. Transcription termination sequences frequently include DNA sequences of about 50 nucleotides capable of forming stem loop structures that aid in terminating transcription. Examples include transcription termination sequences derived from genes with strong promoters, such as the trp gene in E. coli as well as other biosynthetic genes.

Termination Sequences for Use in Mammalian Cells

Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation (Birnstiel et al., Cell, 41:349 (1985); Proudfoot and Whitelaw, “Termination and 3′ end processing of eukaryotic RNA”, in: Transcription and Splicing (eds. B. D. Hames and D. M. Glover) 1988; Proudfoot, Trends Biochem. Sci., 14:105 (1989)). These sequences direct the transcription of an mRNA which can be translated into the polypeptide encoded by the DNA. Examples of transcription terminator/polyadenylation signals include those derived from SV40 (Sambrook et al., “Expression of cloned genes in cultured mammalian cells”, in: Molecular Cloning: A Laboratory Manual, 1989).

Termination Sequences for Use in Yeast and Insect Cells

Transcription termination sequences recognized by yeast are regulatory regions that are usually located 3′ to the translation stop codon. Examples of transcription terminator sequences that may be used as termination sequences in yeast and insect expression systems are well known. (Lopez-Ferber et al., Methods Mol. Biol. 39:25 (1995); King and Possee, The baculovirus expression system. A laboratory guide. Chapman and Hall, London, England (1992); Gregor and Proudfoot, EMBO J., 17:4771 (1998); O'Reilly et al., Baculovirus expression vectors: a laboratory manual. W.H. Freeman & Company, New York, N.Y. (1992); Richardson, Crit. Rev. Biochem. Mol. Biol. 28:1 (1993); Zhao et al., Microbiol. Mol. Biol. Rev., 63:405 (1999)).

II. Nucleic Acid Constructs and Expression Cassettes

Nucleic acid constructs and expression cassettes can be created through use of recombinant methods that are well known. (Sambrook and Russell, Molecular Cloning: A Laboratory Manual, 3rd edition (Jan. 15, 2001) Cold Spring Harbor Laboratory Press, ISBN: 0879695765; Ausubel et al., Current Protocols in Molecular Biology, Green Publishing Associates and Wiley Interscience, NY (1989)). Generally, recombinant methods involve preparation of a desired DNA fragment and ligation of that DNA fragment into a preselected position in another DNA vector, such as a plasmid.

In a typical example, a desired DNA fragment is first obtained by digesting a DNA that contains the desired DNA fragment with one or more restriction enzymes that cut on both sides of the desired DNA fragment. The restriction enzymes may leave a “blunt” end or a “sticky” end. A “blunt” end means that the end of a DNA fragment does not contain a region of single-stranded DNA. A DNA fragment having a “sticky” end means that the end of the DNA fragment has a region of single-stranded DNA. The sticky end may have a 5′ or a 3′ overhang. Numerous restriction enzymes are commercially available and conditions for their use are also well known. (USB, Cleveland, OH; New England Biolabs, Beverly, Mass.). The digested DNA fragments may be extracted according to known methods, such as phenol/chloroform extraction, to produce DNA fragments free from restriction enzymes. The restriction enzymes may also be inactivated with heat or other suitable means. Alternatively, a desired DNA fragment may be isolated away from additional nucleic acid sequences and restriction enzymes through use of electrophoresis, such as agarose gel or polyacrylamide gel electrophoresis. Generally, agarose gel electrophoresis is used to isolate large nucleic acid fragments while polyacrylamide gel electrophoresis is used to isolate small nucleic acid fragments. Such methods are used routinely to isolate DNA fragments. The electrophoresed DNA fragment can then be extracted from the gel following electrophoresis through use of many known methods, such as electoelution, column chromatography, or binding to glass beads. Many kits containing materials and methods for extraction and isolation of DNA fragments are commercially available. (Qiagen, Venlo, Netherlands; Qbiogene, Carlsbad, Calif.).

The DNA segment into which the fragment is going to be inserted is then digested with one or more restriction enzymes. Preferably, the DNA segment is digested with the same restriction enzymes used to produce the desired DNA fragment. This will allow for directional insertion of the DNA fragment into the DNA segment based on the orientation of the complimentary ends. For example, if a DNA fragment is produced that has an EcoRI site on its 5′ end and a BamHI site at the 3′ end, it may be directionally inserted into a DNA segment that has been digested with EcoRI and BamHI based on the complementarity of the ends of the respective DNAs. Alternatively, blunt ended cloning may be used if no convenient restriction sites exist that allow for directional cloning. For example, the restricton enzyme BsaAI leaves DNA ends that do not have a 5′ or 3′ overhang. Blunt ended cloning may be used to insert a DNA fragment into a DNA segment that was also digested with an enzyme that produces a blunt end. Additionally, DNA fragments and segments may be digested with a restriction enzyme that produces an overhang and then treated with an appropriate enzyme to produce a blunt end. Such enzymes include polymerases and exonucleases. Those of skill in the art know how to use such methods alone or in combination to selectively produce DNA fragments and segments that may be selectively combined.

A DNA fragment and a DNA segment can be combined though conducting a ligation reaction. Ligation links two pieces of DNA through formation of a phosphodiester bond between the two pieces of DNA. Generally, ligation of two or more pieces of DNA occurs through the action of the enzyme ligase when the pieces of DNA are incubated with ligase under appropriate conditions. Ligase and methods and conditions for its use are well known in the art and are commercially available.

The ligation reaction or a portion thereof is then used to transform cells to amplify the recombinant DNA formed, such as a plasmid having an insert. Methods for introducing DNA into cells are well known and are disclosed herein.

Those of skill in the art recognize that many techniques for producing recombinant nucleic acids can be used to produce an expression cassette or nucleic acid construct of the invention. These techniques may be used to isolate individual components of an expression cassette of the invention from existing DNA constructs and insert the components into another piece of DNA to construct an expression cassette. Such techniques can also be used to isolate an expression cassette of the invention and insert it into a desired vector to create a nucleic acid construct of the invention. Additionally, open reading frames may be obtained from genes that are available or are obtained from nature. Methods to isolate and clone genes from nature are known. For example, a desired open reading frame may be obtained through creation of a cDNA library from cells that express a desired polypeptide. The open reading frame may then be inserted into an expression cassette of the invention to allow for production of the encoded preselected polypeptide.

Vectors

Vectors that may be used include, but are not limited to, those able to be replicated in prokaryotes and eukaryotes. For example, vectors may be used that are replicated in bacteria, yeast, insect cells, and mammalian cells. Vectors may be exemplified by plasmids, phagemids, bacteriophages, viruses, cosmids, and F-factors. The invention includes any vector into which the expression cassette of the invention may be inserted and replicated in vitro or in vivo. Specific vectors may be used for specific cells types. Additionally, shuttle vectors may be used for cloning and replication in more than one cell type. Such shuttle vectors are known in the art. The nucleic acid constructs may be carried extrachromosomally within a host cell or may be integrated into a host cell chromosome. Numerous examples of vectors are known in the art and are commercially available. (Sambrook and Russell, Molecular Cloning: A Laboratory Manual, 3rd edition (Jan. 15, 2001) Cold Spring Harbor Laboratory Press, ISBN: 0879695765; New England Biolab, Beverly, Mass.; Stratagene, La Jolla, Calif.; Promega, Madision, Wis.; ATCC, Rockville, Md.; CLONTECH, Palo Alto, Calif.; Invitrogen, Carlabad, Calif.; Origene, Rockville, Md.; Sigma, St. Louis, Mo.; Pharmacia, Peapack, N.J.; USB, Cleveland, Ohio). These vectors also provide many promoters and other regulatory elements that those of skill in the art may include within the nucleic acid constructs of the invention through use of known recombinant techniques.

Vectors for Use in Prokayrotes

A nucleic acid construct for use in a prokaryote host, such as a bacteria, will preferably include a replication system allowing it to be maintained in the host for expression or for cloning and amplification. In addition, a nucleic acid construct may be present in the cell in either high or low copy number. Generally, about 5 to about 200, and usually about 10 to about 150 copies of a high copy number nucleic acid construct will be present within a host cell. A host containing a high copy number plasmid will preferably contain at least about 10, and more preferably at least about 20 plasmids. Generally, about 1 to 10, and usually about 1 to 4 copies of a low copy number nucleic acid construct will be present in a host cell. The copy number of a nucleic acid construct may be controlled by selection of different origins of replication according to methods known in the art. Sambrook and Russell, Molecular Cloning: A Laboratory Manual, 3rd edition (Jan. 15, 2001) Cold Spring Harbor Laboratory Press, ISBN: 0879695765.

A nucleic acid construct containing an expression cassette can be integrated into the genome of a bacterial host cell through use of an integrating vector. Integrating vectors usually contain at least one sequence that is homologous to the bacterial chromosome which allows the vector to integrate. Integrations are thought to result from recombinations between homologous DNA in the vector and the bacterial chromosome. For example, integrating vectors constructed with DNA from various Bacillus strains integrate into the Bacillus chromosome (EPO Publ. No. 127 328). Integrating vectors may also contain bacteriophage or transposon sequences.

Extrachromosomal and integrating nucleic acid constructs may contain selectable markers to allow for the selection of bacterial strains that have been transformed. Selectable markers can be expressed in the bacterial host and may include genes which render bacteria resistant to drugs such as ampicillin, chloramphenicol, erythromycin, kanamycin (neomycin), and tetracycline (Davies et al., Ann. Rev. Microbiol., 32: 469 (1978)). Selectable markers may also include biosynthetic genes, such as those in the histidine, tryptophan, and leucine biosynthetic pathways.

Numerous vectors, either extra-chromosomal or integrating vectors, have been developed for transformation into many bacteria. For example, vectors have been developed for the following bacteria: B. subtilis (Palva et al., Proc. Natl. Acad. Sci. USA, 79: 5582 (1982); EPO Publ. Nos. 036 259 and 063 953; PCT Publ. No. WO 84/04541), E. coli (Shimatake et al., Nature, 292:128 (1981); Amann et al., Gene, 40:183 (1985); Studier et al., J. Mol. Biol., 189:113 (1986); EPO Publ. Nos. 036 776, 136 829 and 136 907)), Streptococcus cremoris (Powell et al., Appl. Environ. Microbiol. 54: 655 (1988)); Streptococcus lividans (Powell et al., Appl. Environ. Microbiol., 54:655 (1988)), and Streptomyces lividans (U.S. Pat. No. 4,745,056). Numerous vectors are also commercially available (New England Biolabs, Beverly, Mass.; Stratagene, La Jolla, Calif.).

Vectors for Use in Yeast

Many vectors may be used to construct a nucleic acid construct that contains an expression cassette of the invention and that provides for the expression of a tandem polypeptide in yeast. Such vectors include, but are not limited to, plasmids and yeast artificial chromosomes. Preferably the vector has two replication systems, thus allowing it to be maintained, for example, in yeast for expression and in a prokaryotic host for cloning and amplification. Examples of such yeast-bacteria shuttle vectors include YEp24 (Botstein, et al., Gene, 8:17 (1979)), pC1/1 (Brake et al., Proc. Natl. Acad. Sci. USA, 81:4642 (1984)), and YRp17 (Stinchcomb et al., J. Mol. Biol., 158:157 (1982)). A vector may be maintained within a host cell in either high or low copy number. For example, a high copy number plasmid will generally have a copy number ranging from about 5 to about 200, and usually about 10 to about 150. A host containing a high copy number plasmid will preferably have at least about 10, and more preferably at least about 20. Either a high or low copy number vector may be selected, depending upon the effect of the vector and the tandem polypeptide on the host. (Brake et al., Proc. Natl. Acad. Sci. USA, 81:4642 3(1984)).

A nucleic acid construct may also be integrated into the yeast genome with an integrating vector. Integrating vectors usually contain at least one sequence homologous to a yeast chromosome that allows the vector to integrate, and preferably contain two homologous sequences flanking an expression cassette of the invention. Integrations appear to result from recombinations between homologous DNA in the vector and the yeast chromosome. (Orr-Weaver et al., Methods in Enzymol., 101:228 (1983)). An integrating vector may be directed to a specific locus in yeast by selecting the appropriate homologous sequence for inclusion in the vector. One or more nucleic acid constructs may integrate, which may affect the level of recombinant protein produced. (Rine et al., Proc. Natl. Acad. Sci. USA, 80:6750 (1983)). The chromosomal sequences included in the vector can occur either as a single segment in the vector, which results in the integration of the entire vector, or two segments homologous to adjacent segments in the chromosome and flanking an expression cassette included in the vector, which can result in the stable integration of only the expression cassette.

Extrachromosomal and integrating nucleic acid constructs may contain selectable markers that allow for selection of yeast strains that have been transformed. Selectable markers may include, but are not limited to, biosynthetic genes that can be expressed in the yeast host, such as ADE2, HIS4, LEU2, TRP1, and ALG7, and the G418 resistance gene, which confer resistance in yeast cells to tunicamycin and G418, respectively. In addition, a selectable marker may also provide yeast with the ability to grow in the presence of toxic compounds, such as metal. For example, the presence of CUP 1 allows yeast to grow in the presence of copper ions. (Butt et al., Microbiol. Rev., 51:351 (1987)).

Many vectors have been developed for transformation into many yeasts. For example, vectors have been developed for the following yeasts: Candida albicans (Kurtz et al., Mol. Cell. Biol., 6:142 (1986)), Candida maltose (Kunze et al., J. Basic Microbiol., 25:141 (1985)), Hansenula polymorpha (Gleeson et al., J. Gen. Microbiol., 132:3459 (1986); Roggenkamp et al., Mol. Gen. Genet., 202:302 (1986), Kluyveromyces fragilis (Das et al., J. Bacteriol., 158: 1165 (1984)), Kluyveromyces lactis (De Louvencourt et al., J. Bacteriol., 154:737 (1983); van den Berg et al., Bio/Technology, 8:135 (1990)), Pichia guillerimondii (Kunze et al., J. Basic Microbiol., 25:141 (1985)), Pichia pastoris (Cregg et al., Mol. Cell. Biol., 5: 3376, 1985; U.S. Pat. Nos. 4,837,148 and 4,929,555), Saccharomyces cerevisiae (Hinnen et al., Proc. Natl. Acad. Sci. USA, 75:1929 (1978); Ito et al., J. Bacteriol. 153:163 (1983)), Schizosaccharomyces pombe (Beach and Nurse, Nature, 300:706 (1981)), and Yarrowia lipolytica (Davidow et al., Curr. Genet., 10:39 (1985); Gaillardin et al., Curr. Genet., 10:49 (1985)).

Vectors for Use in Insect Cells

Baculovirus vectors have been developed for infection into several insect cells and may be used to produce nucleic acid constructs that contain an expression cassette of the invention. For example, recombinant baculoviruses have been developed for Aedes aegypti, Autographa californica, Bombyx mori, Drosophila melanogaster, Spodoptera frugiperda, and Trichoplusia ni (PCT Pub. No. WO 89/046699; Carbonell et al., J. Virol. 56:153 (1985); Wright, Nature, 321: 718 (1986); Smith et al., Mol. Cell. Biol., 3: 2156 (1983); and see generally, Fraser et al., In Vitro Cell. Dev. Biol., 25:225 (1989)). Such a baculovirus vector may be used to introduce an expression cassette into an insect and provide for the expression of a tandem polypeptide within the insect cell.

Methods to form a nucleic acid construct having an expression cassette of the invention inserted into a baculovirus vector are well known in the art.

Briefly, an expression cassette of the invention is inserted into a transfer vector, usually a bacterial plasmid which contains a fragment of the baculovirus genome, through use of common recombinant methods. The plasmid may also contain a polyhedrin polyadenylation signal (Miller et al., Ann. Rev. Microbiol., 42:177 (1988)) and a prokaryotic selection marker, such as ampicillin resistance, and an origin of replication for selection and propagation in Esecherichia coli. A convenient transfer vector for introducing foreign genes into AcNPV is pAc373. Many other vectors, known to those of skill in the art, have been designed. Such a vector is pVL985 (Luckow and Summers, Virology, 17:31 (1989)).

A wild-type baculoviral genome and the transfer vector having an expression cassette insert are transfected into an insect host cell where the vector and the wild-type viral genome recombine. Methods for introducing an expression cassette into a desired site in a baculovirus virus are known in the art. (Summers and Smith, Texas Agricultural Experiment Station Bulletin No. 1555, 1987. Smith et al., Mol. Cell. Biol., 3:2156 (1983); and Luckow and Summers, Virology 17:31 (1989)). For example, the insertion can be into a gene such as the polyhedrin gene, by homologous double crossover recombination; insertion can also be into a restriction enzyme site engineered into the desired baculovirus gene (Miller et al., Bioessays 4:91 (1989)). The expression cassette, when cloned in place of the polyhedrin gene in the nucleic acid construct, will be flanked both 5′ and 3′ by polyhedrin-specific sequences. An advantage of inserting an expression cassette into the polyhedrin gene is that occlusion bodies resulting from expression of the wild-type polyhedrin gene may be eliminated. This may decrease contamination of tandem polypeptides produced through expression and formation of occulsion bodies in insect cells by wild-type proteins that would otherwise form occlusion bodies in an insect cell having a functional copy of the polyhedrin gene.

The packaged recombinant virus is expressed and recombinant plaques are identified and purified. Materials and methods for baculovirus and insect cell expression systems are commercially available in kit form. (Invitrogen, San Diego, Calif., USA (“MaxBac” kit)). These techniques are generally known to those skilled in the art and fully described in Summers and Smith, Texas Agricultural Experiment Station Bulletin No. 1555, 1987.

Plasmid-based expression systems have also been developed the may be used to introduce an expression cassette of the invention into an insect cell and produce a tandem polypeptide. (McCarroll and King, Curr. Opin. Biotechnol., 8:590 (1997)). These plasmids offer an alternative to the production of a recombinant virus for the production of tandem polypeptides.

Vectors for Use in Mammalian Cells

An expression cassette of the invention may be inserted into many mammalian vectors that are known in the art and are commercially available. (CLONTECH, Carlsbad, Calif.; Promega, Madision, Wis.; Invitrogen, Carlsbad, Calif.). Such vectors may contain additional elements such as enhancers and introns having functional splice donor and acceptor sites. Nucleic acid constructs may be maintained extrachromosomally or may integrate in the the chromosomal DNA of a host cell. Mammalian vectors include those derived from animal viruses, which require trans-acting factors to replicate. For example, vectors containing the replication systems of papovaviruses, such as SV40 (Gluzman, Cell, 23:175 (1981)) or polyomaviruses, replicate to extremely high copy number in the presence of the appropriate viral T antigen. Additional examples of mammalian vectors include those derived from bovine papillomavirus and Epstein-Barr virus. Additionally, the vector may have two replication systems, thus allowing it to be maintained, for example, in mammalian cells for expression and in a prokaryotic host for cloning and amplification. Examples of such mammalian-bacteria shuttle vectors include pMT2 (Kaufman et al., Mol. Cell. Biol., 9:946 (1989)) and pHEBO (Shimizu et al., Mol. Cell. Biol., 6:1074 (1986)).

III. Cells Containing an Expression Cassette or a Nucleic Acid Construct

The invention provides cells that contain an expression cassette of the invention or a nucleic acid construct of the invention. Such cells may be used for expression of a preselected polypeptide. Such cells may also be used for the amplification of nucleic acid constructs. Many cells are suitable for amplifying nucleic acid constructs and for expressing preselected polypeptides. These cells may be prokaryotic or eukaryotic cells.

In a preferred embodiment, bacteria are used as host cells. Examples of bacteria include, but are not limited to, Gram-negative and Gram-positive organisms. Escherichia coli is a preferred organism for expression of preselected polypeptides and amplification of nucleic acid constructs. Many publically available E. coli strains include K-strains such as MM294 (ATCC 31, 466); X1776 (ATCC 31, 537); KS 772 (ATCC 53, 635); JM109; MC1061; HMS 174; and the B-strain BL21. Recombination minus strains may be used for nucleic acid construct amplification to avoid recombination events. Such recombination events may remove concatamers of open reading frames as well as cause inactivation of an expression cassette. Furthermore, bacterial strains that do not express a select protease may also be useful for expression of preselected polypeptides to reduce proteolytic processing of expressed polypeptides. Such a strain is exemplified by Y1090hsdR which is deficient in the Ion protease.

Eukaryotic cells may also be used to produce a preselected polypeptide and for amplifying a nucleic acid construct. Eukaryotic cells are useful for producing a preselected polypeptide when additional cellular processing is desired. For example, a preselected polypeptide may be expressed in a eukaryotic cell when glycosylation of the polypeptide is desired. Examples of eulcaryotic cell lines that may be used include, but are not limited to: AS52, H187, mouse L cells, NIH-3T3, HeLa, Jurkat, CHO-K1, COS-7, BHK-21, A-431, HEK293, L6, CV-1, HepG2, HC1, MDCK, silkworm cells, mosquito cells, and yeast.

Methods for introducing exogenous DNA into bacteria are well-known in the art, and usually include either the transformation of bacteria treated with CaCl₂ or other agents, such as divalent cations and DMSO. DNA can also be introduced into bacterial cells by electroporation, use of a bacteriophage, or ballistic transformation. Transformation procedures usually vary with the bacterial species to be transformed (Masson et al., FEMS Microbiol. Lett., 60:273 (1989); Palva et al., Proc. Natl. Acad. Sci. USA, 79:5582 (1982); EPO Publ. Nos. 036 259 and 063 953; PCT Publ. No. WO 84/04541 [Bacillus], Miller et al., Proc. Natl. Acad. Sci. USA 8:856 (1988); Wang et al., J. Bacteriol., 172:949 (1990) [Campylobacter], Cohen et al., Proc. Natl. Acad. Sci. USA 69:2110 (1973); Dower et al., Nuc. Acids Res., 16:6127 (1988); Kushner, “An improved method for transformation of Escherichia coli with ColE1-derived plasmids”, in: Genetic Engineering: Proceedings of the International Symposium on Genetic Engineering (eds. H. W. Boyer and S. Nicosia), 1978; Mandel et al., J. Mol. Biol., 53:159 (1970); Taketo, Biochim. Biophys. Acta, 949:318 (1988) [Escherichia], Chassy et al., FEMS Microbiol. Lett., 44:173 (1987) [Lactobacillus], Fiedler et al., Anal. Biochem, 170:38 (1988) [Pseudomonas], Augustin et al., FEMS Microbiol. Lett., 66:203 (1990) [Staphylococcus], Barany et al., J. Bacteriol. 144:698 (1980); Harlander, “Transformation of Streptococcus lactis by electroporation”, in: Streptococcal Genetics (ed. J. Ferretti and R. Curtiss III), 1987; Perry et al., Infec. Imnun., 32:1295 (1981); Powell et al., Appl. Environ. Microbil., 54:655 (1988); Somkuti et al., Proc. 4th Eur. Cong. Biotechnology, 1:412 (1987) [Streptococcus].

Methods for introducing exogenous DNA into yeast hosts are well-known in the art, and usually include either the transformation of spheroplasts or of intact yeast cells treated with alkali cations. Transformation procedures usually vary with the yeast species to be transformed (Kurtz et al., Mol. Cell. Biol., 6:142 (1986); Kunze et al., J. Basic Microbiol., 25:141 (1985) [Candida], Gleeson et al., J. Gen. Microbiol. 132:3459 (1986); Roggencamp et al., Mol. Gen. Genet., 202:302 (1986) [Hansenula], Das et al., J. Bacteriol., 158:1165 (1984); De Louvencourt et al., J. Bacteriol., 754:737 (1983); Van den Berg et al., Bio/Technology, 8:135 (1990) [Kluyveromyces], Cregg et al., Mol. Cell. Biol., 5:3376 (1985); Kunze et al., J. Basic Microbiol., 25:141 (1985); U.S. Pat. Nos. 4,837,148 and 4,929,555 [Pichia], Hinnen et al., Proc. Natl. Acad. Sci. USA, 75:1929 (1978); Ito et al., J. Bacteriol., 153:163 (1983) [Saccharomyces], Beach and Nurse, Nature, 300:706 (1981) [Schizosaccharomyces], and Davidow et al., Curr. Genet., 10:39 (1985); Gaillardin et al., Curr. Genet., 10:49 (1985) [Yarrowia]).

Exogenous DNA is conveniently introduced into insect cells through use of recombinant viruses, such as the baculoviruses described herein.

Methods for introduction of heterologous polynucleotides into mammalian cells are known in the art and include lipid-mediated transfection, dextran-mediated transfection, calcium phosphate precipitation, polybrene-mediated transfection, protoplast fusion, electroporation, encapsulation of -the polynucleotide(s) in liposomes, biollistics, and direct microinjection of the DNA into nuclei. The choice of method depends on the cell being transformed as certain transformation methods are more efficient with one type of cell than another. (Felgner et al., Proc. Natl. Acad. Sci., 84:7413 (1987); Felgner et al., J. Biol. Chem., 269:2550 (1994); Graham and van der Eb, Virology, 52:456 (1973); Vaheri and Pagano, Virology, 27:434 (1965); Neuman et al., EMBO J., 1:841 (1982); Zimmerman, Biochem. Biophys. Acta., 694:227 (1982); Sanford et al., Methods Enzymol., 217:483 (1993); Kawai and Nishizawa, Mol. Cell. Biol., 4:1172 (1984); Chaney et al., Somat. Cell Mol. Genet., 12:237 (1986); Aubin et al., Methods Mol. Biol., 62:319 (1997)). In addition, many commercial kits and reagents for transfection of eukaryotic are available.

Following transformation or transfection of a nucleic acid into a cell, the cell may be selected for through use of a selectable marker. A selectable marker is generally encoded on the nucleic acid being introduced into the recipient cell. However, co-transfection of selectable marker can also be used during introduction of nucleic acid into a host cell. Selectable markers that can be expressed in the recipient host cell may include, but are not limited to, genes which render the recipient host cell resistant to drugs such as actinomycin C₁, actinomycin D, amphotericin, ampicillin, bleomycin, carbenicillin, chloramphenicol, geneticin, gentamycin, hygromycin B, kanamycin monosulfate, methotrexate, mitomycin C, neomycin B sulfate, novobiocin sodium salt, penicillin G sodium salt, puromycin dihydrochloride, rifampicin, streptomycin sulfate, tetracycline hydrochloride, and erythromycin. (Davies et al., Ann. Rev. Microbiol., 32: 469 (1978)). Selectable markers may also include biosynthetic genes, such as those in the histidine, tryptophan, and leucine biosynthetic pathways. Upon transfection or tranformation of a host cell, the cell is placed into contact with an appropriate selection marker.

For example, if a bacterium is transformed with a nucleic acid construct that encodes resistance to ampicillin, the transformed bacterium may be placed on an agar plate containing ampicillin. Thereafter, cells into which the nucleic acid construct was not introduced would be prohibited from growing to produce a colony while colonies would be formed by those bacteria that were successfully transformed. An analogous system may be used to select for other types of cells, including both prokaryotic and eukaryotic cells.

IV. Tandem Polypeptides

The invention provides numerous tandem polypeptides that include a preselected polypeptide operably linked to an inclusion body fusion partner that causes the tandem polypeptide to form inclusion bodies having useful isolation enhancement characteristics. In one embodiment, tandem polypeptides can include an inclusion body fusion partner that is operably linked to a preselected polypeptide. The inclusion body fusion partner may be linked to the amino-terminus or the carboxyl-terminus of the preselected polypeptide. In another embodiment, a tandem polypeptide can have an inclusion body fusion partner operably linked to both the amino-terminus and the carboxyl-terminus of a preselected polypeptide. A tandem polypeptide may also include multiple copies of an inclusion body fusion partner. In other embodiments, a tandem polypeptide can have additional amino acid sequences in addition to an inclusion body fusion partner and a preselected polypeptide. For example, a tandem polypeptide may contain one or more cleavable peptide linkers, fusion tags, and preselected polypeptides. Cleavable peptide linkers can be operably linked between an inclusion body fusion partner and a preselected polypeptide, between a preselected polypeptide and a fusion tag, between multiple copies of a preselected polypeptide, or any combination thereof. Also cleavable peptide linkers that are cleaved by different cleavage agents can be operably linked within a single tandem polypeptide. In additional embodiments, a tandem polypeptide can include one or more fusion tags.

The tandem polypeptide can have numerous preselected polypeptides operably linked to an inclusion body fusion partner. Preferably the preselected polypeptide is a bioactive polypeptide. Examples of such polypeptides are GLP-1, GLP-2, PTH, and GRF.

V. Method to Produce a Tandem Polypeptide

Methods to produce a tandem polypeptide are provided by the invention.

The methods involve using an expression cassette of the invention to produce a tandem polypeptide. A tandem polypeptide can be produced in vitro through use of an in vitro transcription and translation system, such as a rabbit reticulocyte lysate system. Preferably a tandem polypeptide is expressed within a cell into which an expression cassette encoding the tandem polypeptide has been introduced.

Generally, cells having an expression cassette integrated into their genome or which carry an expression cassette extrachromosomally are grown to high density and then induced. Following induction, the cells are harvested and the tandem polypeptide is isolated. Such a system is preferred when an expression cassette includes a repressed promoter. This type of system is useful when a tandem polypeptide contains a preselected polypeptide that is toxic to the cell. Examples of such preselected polypeptides include proteases and other polypeptides that interfere with cellular growth. The cells can be induced by many art recognized methods that include, but are not limited to, heat shift, addition of an inducer such as IPTG, or infection by a virus or bacteriophage that causes expression of the expression cassette.

Alternatively, cells that carry an expression cassette having a constitutive promoter do not need to be induced as the promoter is always active. In such systems, the cells are allowed to grow until a desired quantity of tandem polypeptide is produced and then the cells are harvested.

Methods and materials for the growth and maintenance of many types of cells are well known and are available commercially. Examples of media that may be used include, but are not limited to: YEPD, LB, TB, 2xYT, GYT, M9, NZCYM, NZYM, NZN, SOB, SOC, Alsever's solution, CHO medium, Dulbecco's Modified Eagle's Medium, and HBSS. (Sigma, St. Louis, Mo.; Sambrook and Russell, Molecular Cloning: A Laboratory Manual, 3rd edition (Jan. 15, 2001) Cold Spring Harbor Laboratory Press, ISBN: 0879695765; Summers and Smith, Texas Agricultural Experiment Station Bulletin No. 1555, (1987)).

TABLES

Table I
Amino acid and nucleic acid seciuences of an
inclusion body fusion partner

| | | SEQ ID |
|---|---|---|
| Name | Sequence | NO: |
| IBFP | AEEEEILLEVSLVFKVKEFAPDAPLFTGPAY | 1 |
| IBFP | GCT GAA GAA GAA GAA ATT TTA TTA GAA | 3 |
| | GTT TCT TTA GTT TTT AAA GTT AAA GAA TTT | |
| | GCT CCT GAT GCT CCT TTA TTT ACT GGT CCT | |
| | GCT TAT | |

TABLE II

Amino acid sequences and modifications of
preselected polypeptide examples

| | | |
|---|---|---|
| | | SEQ ID |
| Name | Amino Acid Sequences | NO: |

| | | |
|---|---|---|
| GLP-1(7-36) | HAEGTFTSDVSSYLEGQAAKEFIA | 4 |
| | WLVKGR | |
| GLP-1(7-36) NH₂ | HAEGTFTSDVSSYLEGQAAKEFIA | 4 |
| | WLVKGR-NH2 | |
| GLP-1(7-37) | HAEGTFTSDVSSYLEGQAAKEFIA | 5 |
| | WLVKGRG | |
| GLP-1(7-37) NH₂ | HAEGTFTSDVSSYLEGQAAKEFIA | 5 |
| | WLVKGRG-NH₂ | |
| GLP-1(7-36) K26R | HAEGTFTSDVSSYLEGQAAREFIAW | 6 |
| | LVKGR | |
| GLP-1(7-36) K26R- | HAEGTFTSDVSSYLEGQAAREFIAW | 6 |
| NH₂ | LVKGR-NH₂ | |
| GLP-1(7-37) K26R | HAEGTFTSDVSSYLEGQAAREFIAW | 7 |
| LVKGRG | | |
| GLP-1(7-37) K26R- | HAEGTFTSDVSSYLEGQAAREFIAW | 7 |
| NH₂ | LVKGRG-NH₂ | |
| GLP-2(1-34) | HADGSFSDGMNTILDNLAARDFIN | 8 |
| | WLIQTKITDR | |
| GLP-2(1-34)-NH₂ | HADGSFSDGMNTILDNLAARDFIN | 8 |
| | WLIQTKITDR-NH₂ | |
| GLP-2(1-33) | HADGSFSDGMNTILDNLAARDFIN | 9 |
| | WLIQTKITD | |
| GLP-2(1-33)-NH₂ | HADGSFSDGMNTILDNLAARDFIN | 9 |
| | WLIQTKITD-NH₂ | |
| GLP-2(1-33) A2G | HGDGSFSDGMNTILDNLAARDFIN | 10 |
| | WLIQTKITD | |
| GLP-2(1-33) A2G- | HGDGSFSDGMNTILDNLAARDFIN | 10 |
| NH₂ | WLIQTKITD-NH₂ | |
| GLP-2(1-34) A2G | HGDGSFSDGMNTILDNLAARDFIN | 11 |
| | WLIQTKITDR | |
| GLP-2(1-34) A2G- | HGDGSFSDGMNTILDNLAARDFN | 11 |
| NH₂ | WLIQTKITDR-NH₂ | |
| GRF(1-44) | YADAIFTNSYRKVLGQLSARKLLQ | 12 |
| | DIMSRQQGESNQERGARARL | |
| PTH(1-34) | SVSEIQLMHNLGKHLNSMERVEWL | 13 |
| | RKKLQDVHNF | |
| PTH(1-37) | SVSEIQLMHNLGKHLNSMERVEWL | 14 |
| | RKKLQDVHNFVAL | |
| PTH(1-84) | SVSEIQLMHNLGKHLNSMERVEWL | 15 |
| | RKKLQDVHNFVALGAPLAPRDAGS | |
| | QRPRKKEDNVLVESHEKSLGEADK | |
| | ADVNVLTKAKSQ | |
| Amyloid P | H-Glu-Lys-Pro-Leu-Gln-Asn-Phe-Thr- | 16 |
| Component (27-38) | Leu-Cys-Phe-Arg-NH₂ | |
| Amide | | |
| (Tyr0)-Fibrinopeptide | H-Tyr-Ala-Asp-Ser-Gly-Glu-Gly-Asp- | 17 |
| A | Phe-Leu-Ala-Glu-Gly-Gly-Gly-Val- | |
| | Arg-OH | |
| Urechistachykinin II | H-Ala-Ala-Gly-Met-Gly-Phe-Phe-Gly- | 18 |
| | Ala-Arg-NH₂ | |
| Amyloid β-Protein | H-Val-His-His-Gln-Lys-Leu-Val-Phe- | 19 |
| (12-28) | Phe-Ala-Glu-Asp-Val-Gly-Ser-Asn- | |
| | Lys-OH | |
| Amyloid β-Protein | H-Glu-Asp-Val-Gly-Ser-Asn-Lys-Gly- | 20 |
| (22-35) | Ala-Ile-Ile-Gly-Leu-Met-OH | |
| β-Endorphin (camel) | H-Tyr-Gly-Phe-Met-Thr-Ser-Glu-Lys- | 21 |
| | Ser-Gln-Thr-Pro-Leu-Val-Thr-Leu-Phe- | |
| | Lys-Asn-Ala-Ile-Ile-Lys-Asn-Ala-His- | |
| | Lys-Gly-Gln-OH | |
| Valosin (porcine) | H-Val-Glu-Tyr-Pro-Val-Glu-His-Pro- | 22 |
| | Asp-Lys-Phe-Leu-Lys-Phe-Gly-Met | |
| | Thr-Pro-Ser-Lys-Gly-Val-Leu-Phe-Tyr- | |
| | OH | |
| Vasoactive Intestinal | H-Cys-Ser-Cys-Asn-Ser-Trp-Leu-Asp- | 23 |
| Contractor Peptide | Lys-Glu-Cys-Val-Tyr-Phe-Cys-His | |
| (mouse) | Leu-Asp-Ile-Ile-Trp-OH | |

TABLE III

Nucleic acid sequences of preselected
polypeptide examples

| | | SEQ |
|---|---|---|
| | | ID |
| Name | Nucleic Acid Sequences | NO; |
| GLP-1(7-36) | CAT GCT GAG GGT ACC TTC ACC | 24 |
| | TCC GAC GTT TCC TCC TAC CTG | |
| | GAA GGT CAG GCT GCT AAA | |
| | GAA TTC ATC GCT TGG CTG GTT | |
| | AAA GGT CGT | |
| GLP-1(7-36)-NH₂ | CAT GCT GAG GGT ACC TTC ACC | 24 |
| | TCC GAC GTT TCC TCC TAC CTG | |
| | GAA GGT CAG GCT GCT AAA | |
| | GAA TTC ATC GCT TGG CTG GTT | |
| | AAA GGT CGT | |
| GLP-1(7-37) | CAT GCT GAG GGT ACC TTC ACC | 25 |
| | TCC GAC GTT TCC TCC TAC CTG | |
| | GAA GGT CAG GCT GCT AAA | |
| | GAA TTC ATC GCT TGG CTG GTT | |
| | AAA GGT CGT GGT | |
| GLP-1(7-37)-NH₂ | CAT GCT GAG GGT ACC TTC ACC | 25 |
| | TCC GAC GTT TCC TCC TAC CTG | |
| | GAA GGT CAG GCT GCT AAA | |
| | GAA TTC ATC GCT TGG CTG GTT | |
| | AAA GGT CGT GGT | |
| GLP-1(7-36) K26R | CAT GCT GAG GGT ACC TTC ACC | 26 |
| | TCC GAC GTT TCC TCC TAC CTG | |
| | GAA GGT CAG GCT GCT CGT GAA | |
| | TTC ATC GCT TGG CTG GTT AAA | |
| | GGT CGT | |
| GLP-1(7-36)K26R- | CAT GCT GAG GGT ACC TTC ACC | 26 |
| NH₂ | TCC GAC GTT TCC TCC TAC CTG | |
| | GAA GGT CAG GCT GCT CGT GAA | |
| | TTC ATC GCT TGG CTG GTT AAA | |
| | GGT CGT | |
| GLP-1(7-37)K26R | CAT GCT GAG GGT ACC TTC ACC | 27 |
| | TCC GAC GTT TCC TCC TAC CTG | |
| | GAA GGT CAG GCT GCT CGT GAA | |
| | TTC ATC GCT TGG CTG GTT AAA | |
| | GGT CGT GGT | |
| GLP-1(7-37)K26R- | CAT GCT GAG GGT ACC TTC ACC | 27 |
| NH₂ | TCC GAC GTT TCC TCC TAC CTG | |
| | GAA GGT CAG GCT GCT CGT GAA | |
| | TTC ATC GCT TGG CTG GTT AAA | |
| | GGT CGT GGT | |
| GLP-2(1-34) | CAT CCT GAT GGT TCT TTC TCT | 28 |
| | GAT GAG ATG AAC ACC ATT CTT | |
| | GAT AAT CTT GCC GCC CGT GAC | |
| | TTT ATC AAC TGG TTG ATT CAG | |
| | ACC AAA ATC ACT GAC CGT | |
| GLP-2(1-34)-NH₂ | CAT GCT GAT GGT TCT TTC TCT | 28 |
| | GAT GAG ATG AAC ACC ATT CTT | |
| | GAT AAT CTT GCC GCC CGT GAC | |
| | TTT ATC AAC TGG TTG ATT CAG | |
| | ACC AAA ATC ACT GAC CGT | |
| GLP-2(1-33) | CAT GCT GAT GGT TCT TTC TCT | 29 |
| | GAT GAG ATG AAC ACC ATT CTT | |
| | GAT AAT CTT GCC GCC CGT GAC | |
| | TTT ATC AAC TGG TTG ATT CAG | |
| | ACC AAA ATC ACT GAC | |
| GLP-2(1-33)-NH₂ | CAT GCT GAT GGT TCT TTC TCT | 29 |
| | GAT GAG ATG AAC ACC ATT CTT | |
| | GAT AAT CTT GCC GCC CGT GAC | |
| | TTT ATC AAC TGG TTG ATT CAG | |
| | ACC AAA ATC ACT GAC | |
| GLP-2(1-33)A2G | CAT GGT GAT GGT TCT TTC TCT | 30 |
| | GAT GAG ATG AAC ACC ATT CTT | |
| | GAT AAT CTT GCC GCC CGT GAC | |
| | TTT ATC AAC TGG TTG ATT CAG | |
| | ACC AAA ATC ACT GAC | |
| GLP-2(1-33)A2G- | CAT GGT GAT GGT TCT TTC TCT | 30 |
| NH₂ | GAT GAG ATG AAC ACC ATT CTT | |
| | GAT AAT CTT GCC GCC CGT GAC | |
| | TTT ATC AAC TGG TTG ATT CAG | |
| | ACC AAA ATC ACT GAC | |
| GLP-2(1-34)A2G | CAT GGT GAT GGT TCT TTC TCT | 31 |
| | GAT GAG ATG AAC ACC ATT CTT | |
| | GAT AAT CTT GCC GCC CGT GAC | |
| | TTT ATC AAC TGG TTG ATT CAG | |
| | ACC AAA ATC ACT GAC CGT | |
| GLP-2(1-34)A2G- | CAT GGT GAT GGT TCT TTC TCT | 31 |
| NH₂ | GAT GAG ATG AAC ACC ATT CTT | |
| | GAT AAT CTT GCC GCC CGT GAC | |
| | TTT ATC AAC TCG TTG ATT CAG | |
| | ACC AAA ATC ACT GAC CGT | |
| GRF(1-44) | TAC GCT GAC GCT ATC TTC ACC | 32 |
| | AAC TCT TAC CGT AAA GTT CTG | |
| | GGT CAG CTG TCT GCT CGT AAA | |
| | CTG CTG CAG GAC ATC ATG TCC | |
| | CGT CAG CAG GGT GAA TCT AAC | |
| | CAG GAA CGT GGT GCT CGT GCT | |
| | CGT CTG | |
| PTH(1-34) | TCT GTT TCT GAA ATC CAG C TG | 33 |
| | ATG CAC AAC CTG GGT AAA CAC | |
| | CTG AAC T CT ATG GAA CGT | |
| | GTT GAA TGG CTG CGT AAA AAA | |
| | CTG CAG GA C GTT CAC AAC TTC | |
| PTH(1-37) | TCT GTT TCT GAA ATC CAG C TG | 34 |
| | ATG CAC AAC CTG GGT AAA CAC | |
| | CTG AAC TCT ATG GAA CG T GTT | |
| | GAA TGG CTG CGT AAA AAA CTG | |
| | CAG GAC GTT CAC AAC TTC GTT | |
| | GCT CTG | |
| PTH(1-84) | TCT GTT TCT GAA ATC CAG C TG | 35 |
| | ATG CAC AAC CTG GGT AAA CAC | |
| | CTG AAC T CT ATG GAA CG T | |
| | GTT GAA TGG CTG CGT AAA AAA | |
| | CTG CAG GA C GTT CAC AAC TTC | |
| | GTT GCT CTG GGT GCT CC G CTG | |
| | GCT CCG CGT GAC GCT G GT TCC | |
| | CAG CGT CCG CGT AAA AAA | |
| | GAA GAC A AC GTT CTG GTT | |
| | GAA TCC CAC GAA AAA TCC C | |
| | TG GGT GAA GC T GAC AAA GCT | |
| | GAC GTT AAC GTT CTG ACC AA A | |
| | GCT AAA TCC CAG | |
| Amyloid P | GAA AAA CCG CTG CAG AAC TTC | 36 |
| Component (27-38)- | ACC CTG TGC TTC CGT | |
| NH2 | | |
| (Tyr0)- | TAC GCT GAT TCC GGT GAA GGT | 37 |
| Fibrinopeptide A | GAT TTC CTG GCT GAA GGT GGT | |
| | GGT GTC CGT | |
| Urechistachykinin | GCT GCT GGT ATG GGT TTC TTC | 38 |
| II-NHphd 2 | GGT GCG CGT | |
| Amyloid β-Protein | GTC CAT CAT GAG AAA CTG GTC | 39 |
| (12-28) | TTC TTG CGT GAA GAT GTC GGT | |
| | TCC AAC AAA | |
| Amyloid β-Protein | GAA GAT GTC CGT TCC AAC AAA | 40 |
| (22-35) | GGT GCT ATT ATT GGT CTG ATG | |
| β-Endorphin (camel) | TAC GGT GGT TTC ATG ACC TCC | 41 |
| | GAA AAA TCC CAG ACC CCG CTG | |
| | GTC ACC GTG TTC AAA AAC GCT | |
| | ATT ATT AAA AAC GCT CAT AAA | |
| | AAA GGT CAG | |
| Valosin (porcine) | GTC CAG TAC CCG GTC GAA CAT | 42 |
| CCG GAT AAA TTC CTG AAA TTC | | |
| GGT ATG ACC CCG TCC AAA GGT | | |
| GTC CTG TTC TAC | | |
| Vasoactive | TGC TCC TGC AAC TCC TGG CTG | 43 |
| Intestinal | GAT AAA GAA TGC GTC TAG TTC | |
| Contractor Peptide | TGC CAT GTG GAT ATT ATT TGG | |
| mouse | | |

TABLE IV

Amino acid sequences of cleavable peptide
linkers (CPL)

| | | |
|---|---|---|
| Name | Amino Acid Sequences | SEQ ID NO: |

| | | |
|---|---|---|
| CPL1 | Ala-Phe-Leu-Gly-Pro-Gly-Asp-Arg | 44 |
| CPL2 | Val-Asp-Asp-Arg | 45 |
| CPL3 | Gly-Ser-Asp-Arg | 46 |
| CPL4 | Ile-Thr-Asp-Arg | 47 |
| CPL5 | Pro-Gly-Asp-Arg | 48 |

TABLE V

Nucleic acid sequences of cleavable peptide
linkers (GPL)

| | | SEQ ID |
|---|---|---|
| Name | Nucleic Acid Sequences | NO: |
| CPL1 | GCT TTC CTG GGG CCG GGT GAT CGT | 49 |
| CPL2 | GTC GAC GAT CGT | 50 |
| CPL3 | GGA TCT GAC CGT | 51 |
| CPL4 | ATC ACT GAC CGT | 52 |
| CPL5 | CCG GGT GAC CGT | 53 |

### EXAMPLES

### Example 1

### E. coli Expression Vector pBN121

Preferably, an E. Coli high yield expression vector is present within a cell in high copy number, has a strong promoter contained within the expression cassette, and is stabily maintained. A pBN121 plasmid vector was constructed with consideration to the above preferences. This plasmid uses the larger DNA fragment obtained from a FspI-SmaI digest of pGEX2T (Amersham Pharmacia Biotech, Piscataway, N.J.), a Tac promoter and a kanamycin selection marker. The fragment from pGEX2T contained the origin of replication from pMB1 for high copy number maintenance, the LacIq gene for promoter suppression, the GST terminator for transcription termination and the bla gene for ampicillin resistance. A strong promoter, Tac, was amplified from the pGEX2T plasmid with restriction enzyme sites at both ends using the following primers:

(SEQ ID NO: 54)

Primer 1: 5′ TGC ATT TCT AGA ATT GTG AAT TGT TAT
CCG CTC A 3′

(SEQ ID NO: 55)

Primer 2: 5′ TCA AAG ATC TTA TCG ACT GCA CGG 3′

PCR amplification produced the following product:

(SEQ ID NO: 56)

TCAAAGATCTTATCGACTGCACGGTGCACCAATGCTTCTGGCG
TCAGGCAGCCATCGGAAGCTGTGGTATGGCTGTGCAGGTCGTAAATC
ACTGCATAATTCGTGTCGCTCAAGGCGCACTCCCGTTCTGGATAATGT
TTTTTGCGCCGACATCATAACGGTTCTGGCAAATATTCTGAAATGAGC
TG [symbol] ATTAATCATCGGCTCG [symbol] GTGT[GGAATTGTGAGC
GGATAACAATTC[ACAATTCTAGAAATGCA

The −35 and −10 promoter consensus sequences are bolded and underlined with dots, and the downstream transcriptional start A residue (within the lac operator gene sequence) is bolded and underlined with a solid line. The lac operator sequence is enclosed within brackets. The PCR product of the Tac promoter fragment was ligated with the larger fragment obtained from digestion of pGEX2T with FspI and SmaI. The ligation mixture was transformed into high efficiency E. coli competent cells by heat shock at 42° C. for 45 seconds and streaked on LB +100 μg/ml Ampicillin +Agar plates. Plasmids were prepared from cultures of single colonies of transformed cells. Restriction enzyme digestion was used to confirm construction of a correct plasmid. The XbaI-XhoI fragment from the pET23a plasmid (Novagen, Madison, Wis.), which contained the T7 gene 10 ribosome binding site and the T7tag sequence (MASMT GGQQMGR) (SEQ ID NO: 2), was inserted into the plasmid identified above at the XbaI-SmaI sites. The resulting plasmid was named pBN115(Tac).

To introduce a kanamycin selection marker, the plasmid pBN115(Tac) was digested with AatII and FspI to remove the 0.7 kb ampicillin resistance gene. A 1.1 kb PCR product containing the aminophosphotransferase gene for kanamycin resistance, was cloned into the pBN115(Tac) plasmid at the AatII-FspI sites. The PCR reaction for amplifying the kanamycin resistance gene was performed using the pCR-Blunt plasmid as a template (Invitrogen, Carlsbad, Calif.) and the following primers:

(SEQ ID NO: 57)

KANXY1: 5′-CCT GAC GTC CCG GAT GAA TGT CAG CTA CTG
GGC-3′ (AatII site underlined).

(SEQ ID NO: 58)

KANXY2: 5′-GGC TGC GCA AAG GAG AAA ATA CCG CAT GAG
GAA-3′ (FspI site underlined).

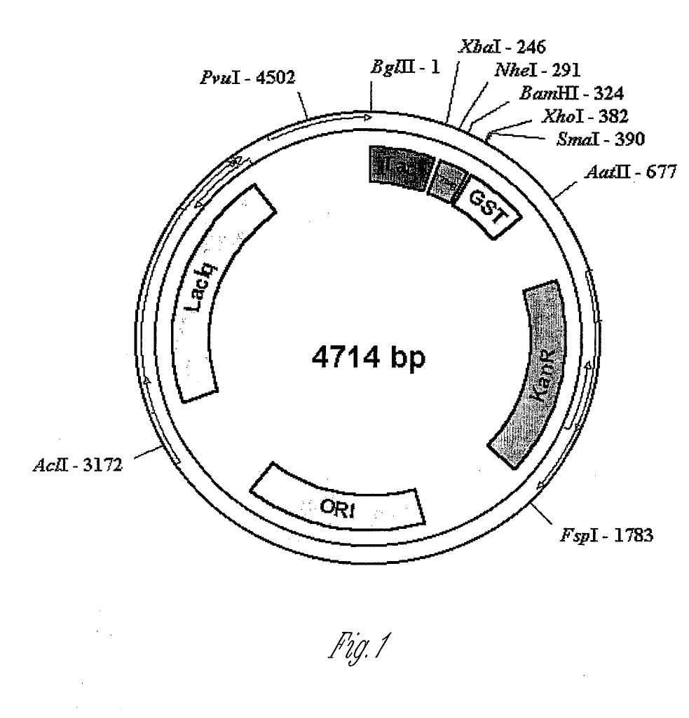

The resulting plasmid was designated as pBN121(Tac). E. coli were transformed with this plasmid and selected in LB+25 μg/ml kanamycin media. Like plasmid pBN115(Tac), pBN121 contains unique NheI and XhoI restriction sites for insertion of a foreign gene sequence that will be expressed. The map of vector pBN121 is shown in FIG. 1.

### Example 2

### Construction of pBN121-T7tagPh-CH-GRP(1-44)CH

Target peptides shorter than 60 amino acids have very low or even lack detectable expression levels in the cytoplasm of E. coli. To overcome this difficulty, methods have been developed to fuse a large polypeptide to a small peptide in order to increase the production of the small peptide. These methods involve the use of large polypeptides that cause the percentage of desired peptide to be small relative to the total protein that is expressed. Short polypeptides (less than 40 amino acids) that allow for the production of small target peptides would allow for the production of small target peptides in greater relative proportion to the amount of total protein expressed within a cell.

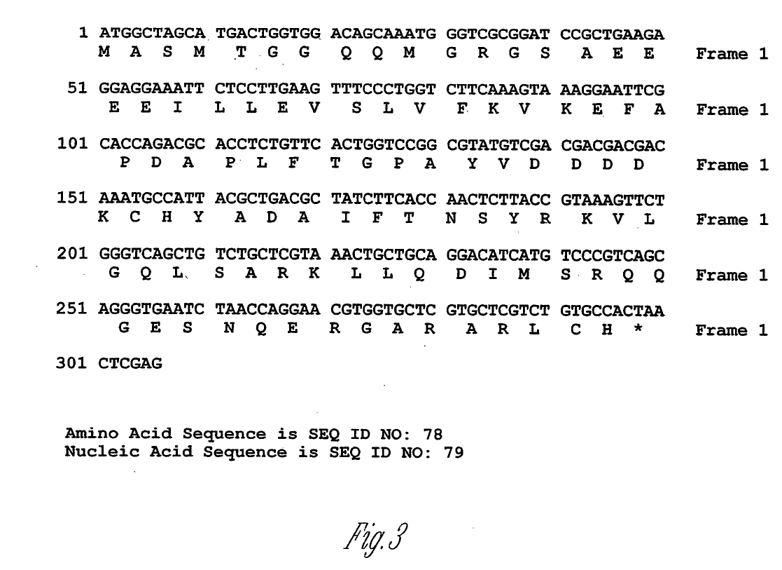

To provide a short (less than 40 amino acid) polypeptide that can be linked to a target peptide and allow the target peptide to be expressed, a short region (Ph) close to the C-terminus of the Autographa californica nucleopolyhedrovirus (AcMNPV) polyhedrin protein (Van Iddekinge et al., Virology 131: 561 (1983)) was isolated. Expression cassettes were designed to incorporate this polyhedrin amino acid sequence, target peptides and modification linkers to promote production of the tandem polypeptide in different E. coli strains (e.g. K strain or B strain). A typical tandem polypeptide expression cassette for a bioactive peptide, GRF(1-44)NH₂, contained the gene sequences for the following: (a) 12 amino acids of the T7tag (MASMTGGQQM GR) (SEQ ID NO: 2); (b) 31 amino acids of the Ph sequence (AEEEE ILLEVSLVFKVKEFAPDAPLFTGPAY) (SEQ ID NO: 1); (c) an amino acid linker (VDDDDKCH) (SEQ ID NO: 59); (d) the target peptide of GRF(1-44); and (e) the C-terminal post-translational modification signal (CH). The sequence of an expression cassette for T7tagPh-CH-GRF(1-44)CH is shown in FIG. 3.

The DNA encoding this Ph region was amplified by PCR extension using the following primers:

(SEQ ID NO: 60)

PH0011A (AGA GGA TCCGCT GAA GAG GAG GAA ATT CTC
CTT GAA GTT TCG CTG GTG TTC AAA),

(SEQ ID NO: 61)

PH0011B (CAG AGG TGC GTC TGG TGC GAA TTC CTT TAC
TTT GAA GAC CAG GGA AAC TTC AAG),

(SEQ ID NO: 62)

PH0011C (GAT GTC GACATA CGC CGG ACC AGT GAA CAG
AGG TGC GTC TGG TGC GAA TTC CTT),

Primer PH0011A and PH0011B anneal to each other. First PCR, using only primer PH0011A and PH0011B produce a short DNA fragment, which could be used as the template for second PCR with primer PH0011A and PH0011C annealed to the template. The second PCR product produced a DNA fragment which encodes the Polyhedrin (215-245) (Ph). The sequence for this DNA fragment is as follows, with BamHI and SalI sites underlined:

(SEQ ID NO: 63)

AGAGGATCCGCTGAAGAGGAGGAAATTCTCCTTGAAGTTTCCC
TGGTCTTCAAAGTAAAGGAATTCGCACCAGACGCACCTCTGTTCACT
GGTCCGGCGTATGTCGACATC.3

The DNA encoding the VDDDDKCH-GRF(1-44)CH was amplified using same PCR extension method as above using synthesized oligonucleotide primers. The PCR product contained the following sequence, with SalI at the N-terminus of VDDDDKCH-GRF(1-44)CH and XhoI sites immediately following the stop codon TAA:

(SEQ ID NO: 64)

GCTATGGTCGACGACGACGACAAATGCCACTACGCTGACGCT
ATCTTCACCAACTCTTACCGTAAAGTTCTGGGTCAGCTGTCTGCTCGT
AAACTGCTGCAGGACATCATGTCCCGTCAGCAGGGTGAATCTAACCA
GGAACGTGGTGCTCGTGCTCGTCTGTGCCACTAACTCGAGCCG

The above two fragments that were obtained from PCR amplification were subjected to SalI digestion, ligated together, and then subjected to BamHI-XhoI digestion. The BamHI-XhoI fragment encoding the Ph-VDDDDKCH-GRF(1-44)CH sequence was inserted into the pBN121plasmid (Example 1) at the sites immediately following the T7tag sequence. The resulted nucleic acid construct was designated as pBN121-T7tagPh-CH-GRF(1-44)CH, its expression cassette is illustrated in FIG. 3. This plasmid was transformed into E. coli HMS173 and BL21 cells. The correct construct was selected in LB+25 μg/ml kanamycin media, and confirmed by restriction enzyme mapping and DNA sequencing. Glycerol stocks of the correct construct were saved at −80° C. or below with 15% glycerol.

### Example 3

### Construction of pBN121-T7tagPh-CPGM-GLP-1(7-36)CHPG and pBN121-T7tagPh-GGGR-GLP- 1(7-36)AFA

The GLP-1(7-36) gene was initially amplified using the same PCR extension method as described in Example 1 using synthesized oligonucleotide primers. The DNA encoding GLP-1(7-36) was cloned into a plasmid vector to be used as a PCR template. Different linker and post-translational modification signals were incorporated with the GLP-1(7-36) sequence by PCR. The DNA encoding the VDCPGM-GLP-1(7-36)CHPG was amplified using this GLP-1(7-36)-containing plasmid as template and using the following synthesized oligonucleotide primers:

(SEQ ID NO: 65)

GLP0009A (GCT ATG GTC GACTGC CCA GGT ATG CAT GCT
GAA GGT ACC TTC ACC TCC),

(SEQ ID NO: 66)

GLP0009B (GTT CTC GAGTTA ACC CGG ATG GCA ACG ACC
TTT AAC CAG CCA AGC GAT)

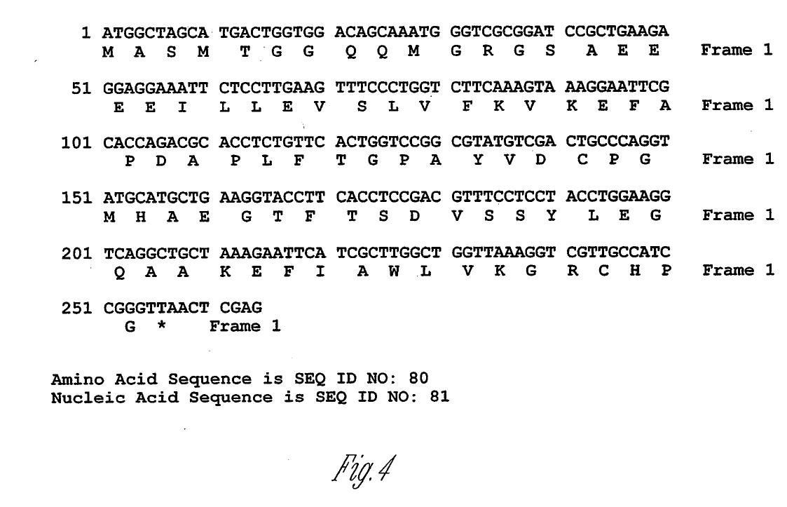

The PCR product had SalI site at the N-terminus of VDCPGM-GLP-1(7-36)CHPG (underlined in primer GLP0009A) and XhoI site immediately following the stop codon TAA (underlined in primer GLP0009B). The PCR product was digested with SalI-XhoI and then inserted into the pBN121-T7tagPh-CH-GRF(1-44)CH nucleic acid construct (Example 2) at the SalI-XhoI sites to replace the CH-GRF(1-44)CH gene. FIG. 4 shows the expression cassette of the resulting nucleic acid construct which was designated pBN121 -T7tagPh-CPGM-GLP-1(7-36)CHPG. This nucleic acid construct was transformed into E. coli HMS173 and BL21 cells. The correct nucleic acid construct was selected and saved as described in Example 2.

The DNA encoding the VDGGGR-GLP-1(7-36)AFA was amplified using the above GLP-1(7-36)-containing nucleic acid construct as template and using the following synthesized oligonucleotide primers:

(SEQ ID NO: 67)

GLP0101A (TAT GTC GACGGT GGT GGT CGT CAT GCT GAA
GGT ACC TTC ACC TCC GAC),

(SEQ ID NO: 68)

GLP0101C (TAA CTC GAGTTA AGC GAA AGC ACG ACC TTT
AAC CAG CCA AGC GAT).

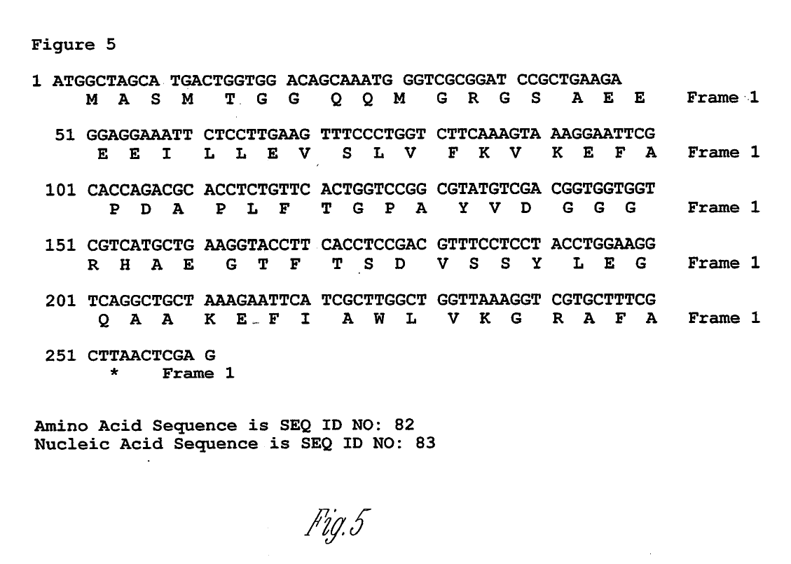

The PCR product had a SalI site at the N-terminus of VDGGGR-GLP-1(7-36)AFA (underlined in primer GLP0101A) and an XhoI site immediately following the stop codon TAA (underlined in primer GLP0101C). The PCR product was digested with SalI and XhoI, and then inserted into the pBN121-T7tagPh-CH-GRF(1-44)CH nucleic acid construct (Example 2) at the SalI and XhoI sites to replace the CH-GRF(1-44)CH gene. FIG. 5 shows the expression cassette of the resulting nucleic acid construct, designated as pBN121-T7tagPh-GGGR-GLP-1(7-36)AFA. This nucleic acid construct was transformed into E. coli HMS 173 and BL21 cells. The correct nucleic acid construct was selected and saved as in Example 2.

Example 4

### Construction of pBN122-M-GLP-1(7-36)-AFAGGGPG-T7tagPh

Other promoters can be easily inserted into the pBN121 plasmid at the BglII and XbaI sites. Several strong promoters were isolated from the Chlorella virus genome (U.S. Pat. No. 6,316,224). One of the Chlorella virus promoters, designated pYX15, was amplified by the PCR extension method as described in Example 2 using synthesized oligonucleotide primers. One PCR extension produced the following pYX15 promoter sequence with restriction enzyme sites flanking:

(SEQ ID NO: 69)

GTTCGAAGATCTAATTCCCGGGGATCAGGCCTCGCTTATAAAT
ATGGTATTGATGTACTTGCCGGTGTGATTGGCTCAGATTACAGAGGA
GAGTTGAAAGCAATCC [symbol] TTGACAATACTACAGAACGTGAC [symbol] TATAATAT
CAAAAAAGTCGACGA[GGAATTGTGAGCGGATAACAATTC]ACAATC
TAGAAAT

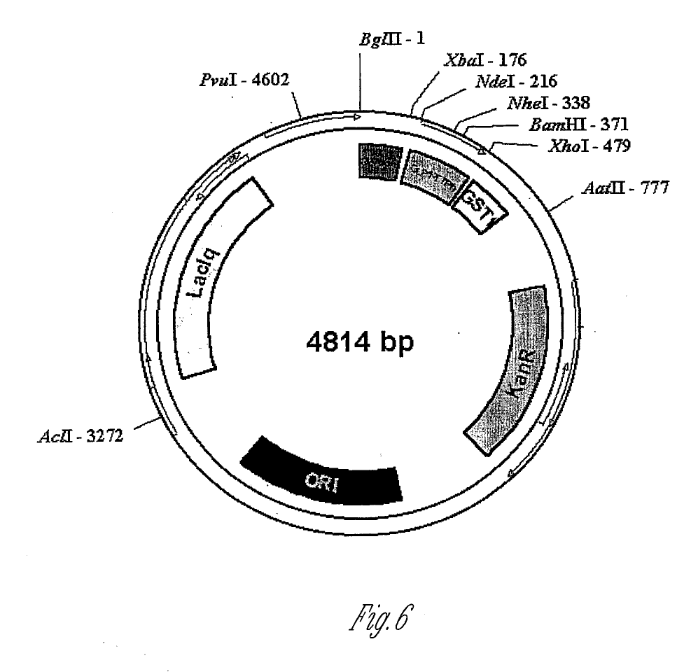

The upstream BglII site (A/GATCT) and the downstream XbaI (T/CTAGA) site sequence are underlined with a single line, the −35 and −10 promoter consensus sequences are indicated as bold and italic. The lac operator sequence is enclosed within brackets. The BglII-XbaI fragment of above PCR product was inserted into the pBN121 plasmid in replacement of the Tac promoter. The resulted plasmid was designated as pBN122, which differed from pBN121 only in the promoter region for the expression cassette. The restriction map and components of a pBN122 nucleic acid construct containing the M-GLP-1(7-36)AFAGGGPG-T7tagPh expression cassette are shown in FIG. 6.

The N-terminus and C-terminus of Ph was changed by PCR using the above Ph containing plasmid as template and the following primers:

(SEQ ID NO: 70)

PH0101A (ATG GCT AGCATG ACT GGT GGA CAG CAA ATG
GGT [symbol] AAA GGA TCC GCT GAA GAG GAG)

(SEQ ID NO: 71)

PH0101B (TAT CAC TCG AGA [symbol] TTA GTC GAC ATA CGC CGG
ACC AGT GAA CAG AGG)

The PCR product encoding T7tagPh-VD had an NheI site at the N-terminus, and the Arg residue in the T7tag was changed to a Lys (bold and italic in primer PH0101A). The Val-Asp at the C-terminus were encoded by DNA corresponding to the SalI site, and was immediately followed by the stop codon TAA (bold and italic in primer PH0101B) and the XhoI site (underlined in primer PH0101B). The PCR product was digested with NheI and XhoI and inserted into the pBN122 plasmid at the same sites. The resulting plasmid was designated as pBN122-T7tagPh.

The N-terminus and C-terminus of GLP-1(7-36) were changed by PCR using the above GLP-1(7-36) containing nucleic acid construct as template and the following primers:

(SEQ ID NO: 72)

GLP0101D (ATG GCT AGC CAT ATGCAT GCT GAA GGT ACC
TTC ACC TCC GAG GTT),

(SEQ ID NO: 73)

GLP0101E (CAT GCT AGCCAT ACC TGG ACC ACC ACC AGC
GAA AGC ACG ACC TTT AAC CAG CCA)

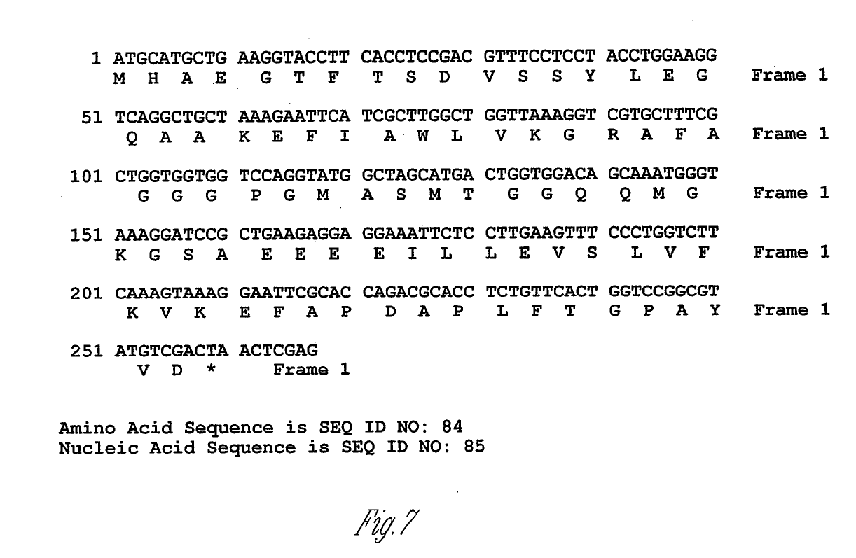

The PCR product encoded M-GLP-1(7-36)AFAGGGPG, where M was encoded by the initiation codon ATG. Cloning sites NdeI-NheI are underlined in the primer sequences above. The PCR product was digested with NdeI and NheI and inserted into the above pBN122-T7tagPh plasmid at the same sites. FIG. 7 illustrated the expression cassette of the resulting nucleic acid construct, designated as pBN122-M-GLP-1(7-36)AFAGGGPG-T7tagPh. This nucleic acid construct was transformed into E. coli BL21 cells. The correct nucleic acid construct was selected and saved as in Example 2.

Example 5

### Construction of pBN121-T7tagPh-VDDR-GLP-2(1-33)A2G

The DNA fragment encoding VDDR-GLP-2(1-33, A2G) was amplified by PCR extension method as in Example 2 using synthesized oligonucleotide primers. The following sequence was obtained:

(SEQ ID NO: 74)

ATGGTCGACGATCGTCATGGTGATGGTTCTTTCTCTGATGAGA
TGAACACCATTCTTGATAATCTTGCCGCCCGTGACTTTATCAACTGGT
TGATTCAGACCAAAATCACTGACTAATAACTCGAGGAA

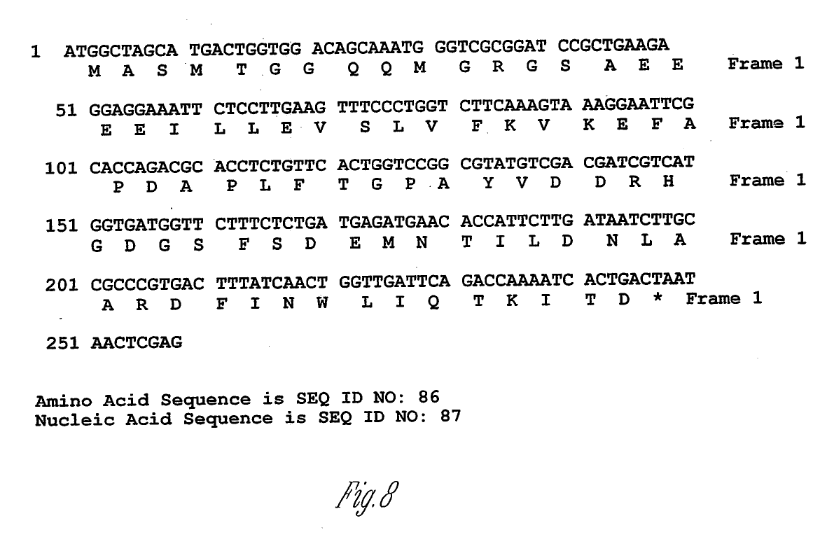

This PCR product had a SalI site (underlined) at the N-terminus and an XhoI site after the stop codon TAA. The fragment was digested with SalI and XhoI and inserted into the pBN121-T7tagPh-CH-GRF(1-44)CH nucleic acid construct (Example 2) at the SalI and XhoI sites to replace the CH-GRF(1-44)CH gene. The resulting nucleic acid construct was designated as pBN121-T7tagPh-GGGR-GLP-1(7-36)AFA, and its expression cassette sequence is illustrated in FIG. 8. This nucleic acid construct was transformed into E. coli BL21 cells. The correct nucleic acid construct was selected and saved as in Example 2.

Example 6

### E. coli Shaking Culture Expression

LBK media (LB+25 μg/ml kanamycin) were used when expressing the constructs using the pBN121 or pBN122 expression vectors. Shaking flask cultures of 5 ml LBK media were started from single colonies of the transformed cells. Shaking flask cultures in 5 ml to 500 ml LBK media (inoculated by 100 μto 10 ml overnight culture) were grown at 37° C. (or other temperatures) and 220 rpm to an A₆₀₀ of 0.5-1.0. Polypeptide expression was then induced by addition of IPTG (1-2 mM final concentration). Cultures were induced for 2 to 8 hours. Samples were taken from flasks containing pre-induced and post-induced cells. Following induction, cells were pelleted and then lysed in 10 mM Tris, 1 mM EDTA, pH 8 by sonication. The samples were centrifuged to separate insoluble and soluble proteins.

Samples of soluble and insoluble proteins were obtained for analysis by SDS-PAGE. The supernatant (soluble protein) from the cell lysate was mixed 1:1 with 2×SDS-PAGE sample buffer. The insoluble proteins were obtained from pellets that were resuspended directly in 1×SDS-PAGE sample. Alternatively, the lysate from induced cells without centrifugation was mixed 1:1 with 2×SDS-PAGE sample buffer. These samples were resolved on SDS-AGE PAGE (Invitrogen) according to manufacturer's instructions and stained with Coomassie Brilliant Blue.

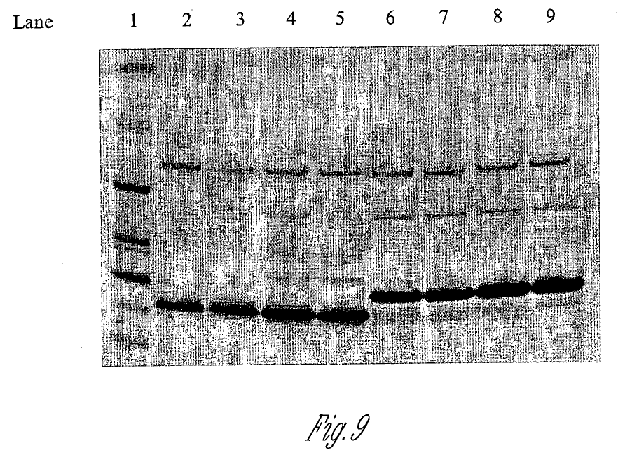

FIG. 9 illustrated that pBN121-T7tagPh-CH-GRF(1-44)CH in E. coli HMS174 and BL21 both produced high level of polypeptides inclusion bodies of T7tagPh-CH-GRF(1-44)CH. This figure also illustrates that pBN121-T7tagPh-CPGM-GLP-1(7-36)CPGM in E. coli HMS174 and BL21 both produced high levels of inclusion bodies containing T7tagPh-CPGM-GLP-1(7-36)CPGM. The T7tagPh-CH-GRF(1-44)CH polypeptide and the T7tagPh-CPGM-GLP-1(7-36) CPGM polypeptide were expressed as inclusion bodies inside E. coli and isolated by centrifugation. They could be solublized in 2 M urea which provides for convenient down stream processing that can be initiated through use of this method.

E. coli BL21 cells with the pBN121-T7tagPh-GGGR-GLP-1(7-36)AFA nucleic acid construct also produced a high level of inclusion bodies of T7tagPh-GGGR-GLP-1(7-36)AFA. This results indicates that linker changes did not affect the expression yield of the polypeptide. The polypeptide inclusion bodies were recovered by centrifugation of the induced cell lysate. The inclusion bodies were then solublized in 8 M urea, diluted 10 fold into buffer (1.5 M NH₃, pH10, 1 mM CaCl₂, 1 mM Cysteine), combined with 20 units of clostripain per mg of polypeptide, and incubated at 45° C. for about 40 minutes. GLP-1(7-36NH₂ was produced in this one pot reaction, which combined cleavage at the N-terminus of GLP-1(7-36) and amidation at the C-terminus.

Surprisingly, BL21 cells with the pBN122-M-GLP-1(7-36)AFAGGGPG-T7tagPh nucleic acid construct produced inclusion bodies of M-GLP-1(7-36)AFAGGGPG-T7tagPh in which Ph was fused to the C-terminus of the target peptide.

BL21 cells with the pBN121-T7tagPh-VDDR-GLP-2(1-33, A2G) nucleic acid construct expressed high level of inclusion bodies of T7tagPh-VDDR-GLP-2(1-33, A2G). The induced cells were able to be lysed in 8 M urea and the polypeptide inclusion bodies were solublized. The polypeptide solution was diluted 10 fold into buffer (50 mM HEPES, pH 6.9, 1 mM CaCl₂, 1 mM Cysteine), combined with about 0.5 unit clostripain per mg of polypeptide, and incubated at 25° C. for about 50 minutes. GLP-2(1-33, A2G) was produced efficiently with minimal degradation.

### Example 7

### E. coli Fermentation Production of Polypeptides

Fermentation expression of the E. coli BL21 cells transformed with pBN121 or pBN122 based nucleic acid constructs were evaluated for polypeptide expression in 5 L or larger fermentation. 100 μl of a glycerol stock of cells containing the nucleic acid construct were used to inoculate 100 ml LB +25 μg/ml Kanamycine media in a shaking flask. The shaking culture was grown in a rotary shake at 37° C. until the A₅₄₀ reached 1.5±0.5. The contents of the shaking flask culture were then used to inoculate a 5 L fermentation tank containing a defined minimal media (e.g. M9 media, Molecular Cloning, 2^{nd} edition, Sambrook et al). Glucose served as the carbon source and was maintained at a concentration below 4%. About 25 μg/ml kanamycin was used in the fermentation. Dissolved oxygen was controlled at 40% by cascading agitation and aeration with additional oxygen. Ammonium hydroxide solution was fed to control the pH at about 6.9 and to supply additional nitrogen. The cells were induced with a final concentration of 0.1-1 mM IPTG after the A₅₄₀ reached 50-75 for 2-6 hours. After the induction was complete, the cells were cooled and harvested by centrifugation. The cell sediments were saved below −20° C. until used or lysed immediately. Cells, after being thawed if they were frozen, are resuspended in distilled water, then lysed by sonication or homogenization. The lysate was centrifuged to pellet down inclusion bodies of the expressed polypeptide. The polypeptide sediments can be dissolved in 8M urea or other solvent for further treatment. More than 4 g of the desired polypeptide could be obtained from 1 liter of fermentation broth.

### Example 8

### Expression of pBN121-M-PTH(1-84), pBN121-T7tag-GSDDDDKCH-PTH(1-84) and pBN121-T7tagPh-VDDDDKCH-PTH( 1-84)

Some target peptides have more than 60 amino acids. It is possible for E. coli to express this kind of target peptides in the E. coli cytoplasm without the use of a fusion partner. However, because many of these peptides are not stable inside E. coli, only low level production could be achieved without the use of a fusion partner. A typical example in this category was PTH(1-84). To overcome the lack of production of PTH(1-84), M-PTH(1-84), T7tag-VDDDDKCH-PTH(1-84) and T7tagPh-VDDDDKCH-PTH(1-84) were cloned and demonstrated to increase the production of the PTH polypeptide by use of the Ph inclusion body fusion partner.

A DNA fragment encoding M-PTH(1-84) was amplified by the PCR extension method described in Example 2 using synthesized oligonucleotide primers. The following sequence was obtained:

(SEQ ID NO: 75)

ATACCACATATGTCTGTTTCTGAAATCCAGCTGATGCACAACC
TGGGTAAACACCTGAACTCTATGGAACGTGTTGAATGGCTGCGTAAA
AAACTGCAGGACGTTCACAACTTCGTTGCTCTGGGTGCTCCGTGGCT
CCGCGTGACGCTGGTTCCCAGCGTCCGCGTAAAAAAGAAGACAACGT
TCTGGTTGAATCCCACGAAAAATCCCTGGGTGAAGCTGACAAAGCTG
ACGTTAACGTTCTGACCAAAGCTAAATCCCAGTAACTCGAGTAT

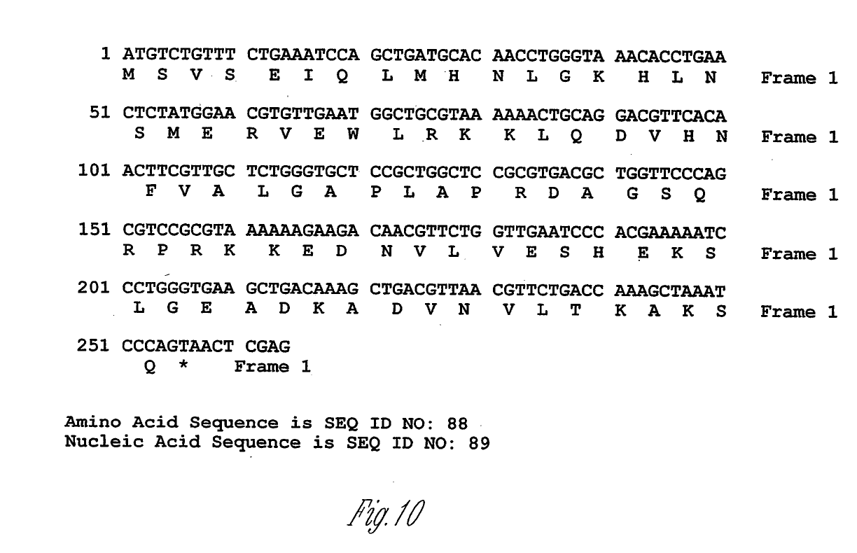

Indicated as underlined, this PCR product had an NdeI site before the starting codon ATG and an A7XhoI site after the stop codon TAA. The fragment was digested with NdeI and XhoI and inserted into the pBN121-T7tagPh-CH-GRF(1-44)CH nucleic acid construct (Example 2) at the NdeI and the XhoI sites to replace the T7tagPh-CH-GRF(1-44)CH gene. The resulting nucleic acid construct was designated pBN121-M-PTH(1-84), where M was encoded by the starting codon ATG. The expression cassette sequence was illustrated in FIG. 10.

The following DNA fragment encoding GSDDDDKCH-PTH(1-84) was amplified by PCR extension:

(SEQ ID NO: 76)

TATGGATTCGACGACGACAAATGCCACTCTGTTTCTGAAATCC
AGCTGATGCACAACCTGGGTAAACACCTGAACTCTATGGAACGTGTT
GAATGGCTGCGTAAAAAACTGCAGGACGTTCACAACTTCGTTGCTCT
GGGTGCTCCGCTGGCTCCGCGTGACGCTGGTTCCCAGCGTCCGCGTA
AAAAAGAAGACAACGTTCTGGTTGAATCCCACGAAAAATCCCTGGGT
GAAGCTGACAAAGCTGACGTTAACGTTCTGACCAAAGCTAAATCCCA
GTAACTCGAGTAT

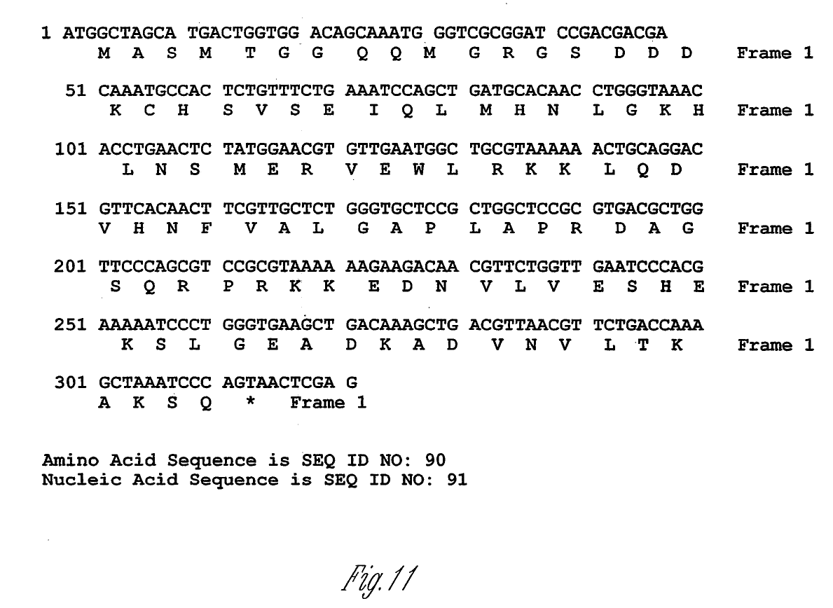

This PCR product had a BamHI site (underlined) at the N-terminus and an XhoI site after the stop codon TAA. The fragment was digested with BamHI and XhoI and inserted into the pBN121-T7tagPh-CH-GRF(1-44)CH nucleic acid construct (Example 2) at the BamHI and the XhoI sites to replace the Ph-CH-GRF(1-44)CH gene. The resulting nucleic acid construct was designated pBN121-T7tag-GSDDDDKCH-PTH(1-84), and its expression cassette sequence is illustrated in FIG. 11.

The DNA fragment encoding VDDDDKCH-PTH(1-84) was amplified by the PCR extension method described in Example 2 using synthesized oligonucleotide primers. The following sequence was obtained:

(SEQ ID NO: 77)

TATGTCGACGACGACGACAAATGCCACTCTGTTTCTGAAATCC
AGCTGATGCACAACCTGGGTAAACACCTGAACTCTATGGAACGTGTT
GAATGGCTGCGTAAAAAACTGCAGGACGTTCACAACTTCGTTGCTCT
GGGTGCTCCGCTGGCTCCGCGTGACGCTGGTTCCCAGCGTCCGCGTA
AAAAAGAAGACAACGTTCTGGTTGAATCCCACGAAAAATCCCTGGGT
GAAGCTGACAAAGCTGACGTTAACGTTCTGACCAAAGCTAAATCCCA
GTAACTCGAGTAT

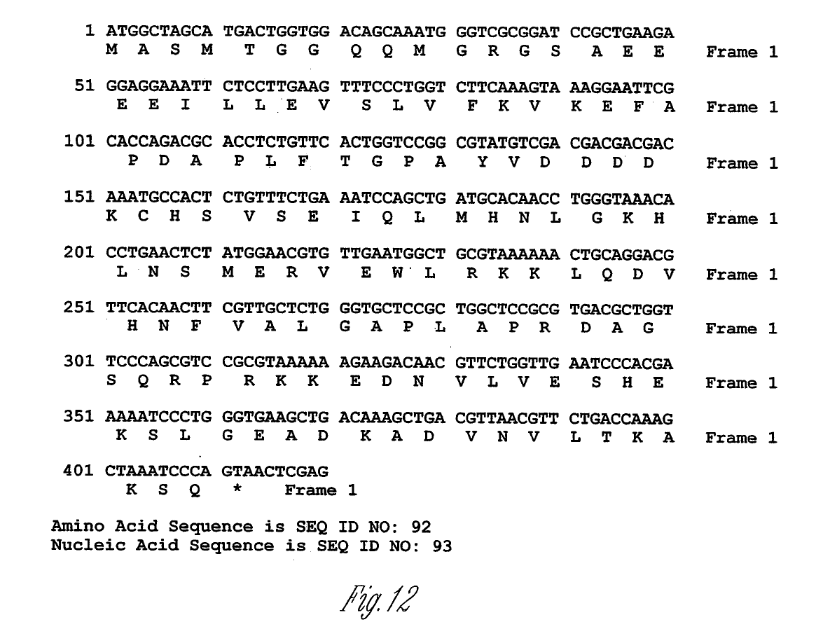

This PCR product had a SalI site (underlined) at the N-terminus and an XhoI site at the stop codon TAA. The fragment was digested with SalI and XhoI and inserted into the pBN121-T7tagPh-CH-GRF(1-44)CH nucleic acid construct (Example 2) at the SalI and XhoI sites to replace the CH-GRF(1-44)CH gene. The resulting nucleic acid construct was designated pBN121-T7tagPh-VDDDDKCH-PTH(1-84), and its expression cassette sequence is illustrated in FIG. 12.

All three nucleic acid constructs above were transformed into E. coli BL21 cells. The correct constructs were selected in LB+25 μg/ml kanamycin, the nucleic acid constructs were confirmed by restriction enzyme mapping and sequencing.

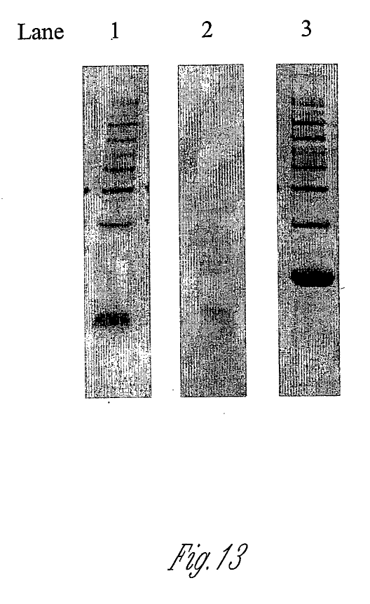

Expression of the above three nucleic acid constructs according to same procedure as described in Example 6 produced vary different yields, with pBN121-T7tagPh-VDDDDKCH-PTH(1-84)/BL21 producing much more polypeptide than the other two constructs given the same quantity of cells (FIG. 13).

References

Alberts et al., Molecular Biology of the Cell, 2 (1989).

Amann et al., Gene, 25:167 (1983).

Amann et al., Gene, 40:183 (1985).

Aubin et al., Methods Mol. Biol., 62:319 (1997).

Augustin et al., FEMS Microbiol. Lett., 66:203 (1990).

Ausubel et al., Current Protocols in Molecular Biology, Green Publishing Associates and Wiley Interscience, NY. (1989).

Barany et al., J. Bacteriol., 144:698 (1980).

Beach and Nurse, Nature, 300:706 (1981).

Beaucage and Caruthers, Tetra. Letts., 22:1859 (1981).

Birnstiel et al., Cell, 41:349 (1985).

Boshart et al., Cell, 41: 521 (1985).

Botstein, et al., Gene, 8:17 (1979).

Brake et al., Proc. Natl. Acad. Sci. USA, 81:4642 (1984).

Butt et al., Microbiol. Rev., 51:351 (1987).

Carbonell et al., Gene, 73: 409 (1988).

Carbonell et al., J. Virol., 56:153 (1985).

Chaney et al., Somat. Cell Mol. Genet., 12:237 (1986).

Chang et al., Nature, 198:1056 (1977).

Chassy et al., FEMS Microbiol. Left. 44:173 (1987).

Cohen et al., Proc. Natl. Acad. Sci. USA, 69:2110 (1973).

Cohen et al., Proc. Natl. Acad. Sci. USA, 77: 1078 (1980).

Coombs et al., Chem. Biol., 5:475 (1998).

Cregg et al., Mol. Cell. Biol. 5: 3376, (1985).

Das et al., J. Bacteriol., 158: 1165 (1984).

Davidow et al., Curr. Genet. 10:39 (1985).

Davies et al., Ann. Rev. Microbiol., 32: 469 (1978).

Dayhoff et al., Atlas of Protein Sequence and Structure (1978) (Natl. Biomed. Res. Found., Washington, D.C.).

de Boer et al., Proc. Natl. Acad. Sci. USA, 80: 21 (1983).

De Louvencourt et al., J. Bacteriol., 154:737 (1983).

De Louvencourt et al., J. Bacteriol., 754:737 (1983).

Dijkema et al., EMBO J., 4:761 (1985.

Dower et al., Nuc. Acids Res. 16:6127 (1988).

Feigner et al., J. Biol. Chem., 269:2550 (1994).

Felgner et al., Proc. Natl. Acad. Sci., 84:7413 (1987).

Fiedler et al., Anal. Biochem, 170:38 (1988).

Franke and Hruby, J. Gen. Virol., 66:2761 (1985).

Fraser et al., In Vitro Cell. Dev. Biol., 25:225 (1989).

Gaillardin et al., Curr. Genet., 10:49 (1985).

Ghrayeb et al., EMBO J., 3: 2437 (1984).

Gleeson et al., J. Gen. Microbiol., 132:3459 (1986).

Gluzman, Cell, 23:175 (1981).

Goeddel et al., N.A.R., 8: 4057 (1980).

Gorman et al., Proc. Natl. Acad. Sci. USA, 79:6777 (1982b).

Graham and van der Eb, Virology, 52:456 (1973).

Gregor and Proudfoot, EMBO J., 17:4771 (1998).

Guan et al., Gene, 67:21 (1997).

Harlander, “Transformation of Streptococcus lactis by electroporation”, in: Streptococcal Genetics (ed. J. Ferretti and R. Curtiss III) 1987.

Harlow and Lane, Antibodies: A Laboratory Manual, Cold Spring Harbor Laboratory Press, Cold Spring Harbor, N.Y. (1988).

Henikoff et al., Nature 283:835 (1981).

Hinnen et al., Proc. Natl. Acad, Sci. USA, 75:1929 (1978).

Hollenberg et al., “The Expression of Bacterial Antibiotic Resistance Genes in the Yeast Saccharomyces cerevisiae”, in: Plasmids of Medical, Environmental and Commercial Importance (eds. K. N. Timmis and A. Puhler), 1979.

Hollenberg et al., Curr. Tonics Microbiol. Immunol., 96: 119 (1981).

Ito et al., J. Bacteriol., 153:163 (1983).

JPO Publ. No. 62,096,086.

Kaufman et al., Mol. Cell. Biol., 9:946 (1989).

Kawai and Nishizawa, Mol. Cell. Biol., 4:1172 (1984).

Kohrer et al., Proc. Natl. Acad. Sci. USA, 98:14310 (2001).

Kowal et al., Proc. Natl. Acad. Sci. (USA), 98:2268 (2001).

Kunkel et al., Methods in Enzymol., 154:367 (1987).

Kunkel, Proc. Natl. Acad. Sci. USA, 82:488, (1985).

Kunze et al., J. Basic Microbiol., 25:141 (1985).

Kurtz et al., Mol. Cell. Biol., 6:142 (1986).

Lebacq-Verheyden et al., Mol. Cell. Biol., 8: 3129 (1988).

Lewin, Genes VII, Oxford University Press, New York, N.Y. (2000).

Lopez-Ferber et al., Methods Mol. Biol., 39:25 (1995).

Luckow and Summers, Virology, 17:31 (1989).

Maeda et al., Nature, 315:592 (1985).

Mandel et al., J. Mol. Biol., 53:159 (1970).

Maniatis et al., Science, 236:1237 (1987).

Martin et al., DNA, 7: 99 (1988).

Marumoto et al., J. Gen. Virol., 68:2599 (1987).

Masson et al., FEMS Microbiol. Lett., 60:273 (1989).

Masui et al., in: Experimental Manipulation of Gene Expression, (1983).

McCarroll and King, Curr. Opin. Biotechnol., 8:590 (1997).

Mercerau-Puigalon et al., Gene, 11:163 (1980).

Miller et al., Ann. Rev. Microbiol. 42:177 (1988).

Miller et al., Bioessays, 4:91 (1989).

Miller et al., Proc. Natl. Acad. Sci. USA, 8:856 (1988).

Miyajima et al., Gene. 58: 273 (1987).

Myanohara et al., Proc. Natl. Acad. Sci. USA, 80: 1 (1983).

Neuman et al., EMBO J., 1:841 (1982).

Oka et al., Proc. Natl. Acad. Sci. USA 82: 7212 (1985).

Orr-Weaver et al., Methods in Enzymol., 101:228 (1983).

Palva et al., Proc. Natl. Acad. Sci. USA, 79: 5582 (1982).

Panthier et al., Curr. Genet., 2:109 (1980).

PCT Publ. No. WO 84/04541.

Pearson, Genomics, 11:635 (1991).

Perry et al., Infec. Immun., 32:1295 (1981).

Powell et al., Appl. Environ. Microbiol., 54: 655 (1988).

Raibaud et al., Ann. Rev. Genet., 18:173 (1984).

Richardson, Crit. Rev. Biochem. Mol. Biol., 28:1 (1993).

Rine et al., Proc. Natl. Acad. Sci. USA, 80:6750 (1983).

Roggenkamp et al., Mol. Gen. Genet. 202:302 (1986).

Sambrook and Russell, Molecular Cloning: A Laboratory Manual, 3rd edition (Jan. 15, 2001) Cold Spring Harbor Laboratory Press, ISBN: 0879695765.

Sambrook et al., “Expression of Cloned Genes in Mammalian Cells”, in: Molecular Cloning: A Laboratory Manual, 2nd ed., 1989.

Sanford et al., Methods Enzymol. 217:483 (1993).

Sassone-Corsi and Borelli, Trends Genet., 2:215 (1986).

Shimatake et al., Nature, 292:128 (1981).

Shimizu et al., Mol. Cell. Biol., 6:1074 (1986).

Shine et al., Nature, 254: 34 (1975).

Smith & Waterman, J. Mol. Biol., 147:195 (1981).

Smith et al., Mol. Cell. Biol., 3: 2156 (1983).

Smith et al., Proc. Natl. Acad. Sci. USA, 82: 8404 (1985).

Somkuti et al., Proc. 4th Eur. Cong. Biotechnology, 1:412 (1987).

Steitz et al., “Genetic signals and nucleotide sequences in messenger RNA”, in: Biological Regulation and Development: Gene Expression (ed. R. F. Goldberger) (1979).

Stinchcomb et al., J. Mol. Biol., 158:157 (1982).

Studier et al., J. Mol. Biol., 189:113 (1986).

Tabor et al., Proc. Natl. Acad. Sci. USA, 82:1074 (1985).

Taketo, Biochim. Biophys. Acta, 949:318 (1988).

Vaheri and Pagano, Virology 27:434 (1965).

Van den Berg et al., Bio/Technology, 8:135 (1990).

Van den Berg et al., Bio/Technology 8:135 (1990).

Van Iddekinge et al., Virology, 131: 561 (1983).

VanDevanter et al., Nucleic Acids Res., 12:6159 (1984).

Vlak et al., J. Gen. Virol., 69: 765 (1988).

Walker and Gaastra, eds. (1983) Techniques in Molecular Biology (MacMillan Publishing Company, New York).

Walsh, Proteins Biochemistry and Biotechnology, John Wiley & Sons, LTD., West Sussex, England (2002).

Wang et al., J. Bacteriol., 172:949 (1990).

Waterman, Bulletin of Mathematical Biology, 46:473 (1984).

Watson, Molecular Biology of the Gene, 4th edition, Benjamin/Cummings Publishing Company, Inc., Menlo Park, Calif. (1987).

Weissmann, “The cloning of interferon and other mistakes”, in: Interferon 3 (ed. I. Gresser), 1981.

Wright, Nature, 321: 718 (1986).

Yelverton et al., N.A.R., 2: 731 (1981).

Zhao et al., Microbiol. Mol. Biol. Rev. 63:405 (1999).

Zimmerman, Biochem. Biophys. Acta., 694:227 (1982).

EPO Publ. No. 012 873.

EPO Publ. No. 060 057.

EPO Publ. No. 164 556.

EPO Publ. No.267 851.

EPO Publ. No. 284 044.

EPO Publ. No. 329 203.

EPO Publ. No. 036 259.

EPO Publ. No. 063 953.

EPO Publ. No. 036 776.

EPO Publ. No. 121 775.

U.S. Pat. No. 4,551,433.

U.S. Pat. No. 4,588,684.

U.S. Pat. No.4,689,406.

U.S. Pat. No. 4,738,921.

U.S. Pat. No. 4,837,148.

U.S. Pat. No.4,929,555.

U.S. Pat. No. 4,837,148.

U.S. Pat. No.4,929,555.

U.S. Pat. No. 4,876,197.

U.S. Pat. No. 4,880,734.

U.S. Pat. No. 4,336,336.

U.S. Pat. No. 6,316,224.

US Pat. No. 4,873,192.

All publications, patents and patent applications including priority patent application Ser. No. 60/383,212 filed on May 24, 2002 are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. An expression cassette comprising the following operably linked nucleic acid sequence:
5′ Pr-(TIS)_{D}-(IBFP1)_{E}-(CL1)_{G}-ORF-[CL2-ORF]_{L}-(CL3)_{M}-(IBFP2)_{Q}-(SSC)_{R}-(CL4)_{T}-(Ft)_{W}-(Tr)_{X}-3′
wherein
Pr is a promoter sequence,
TIS encodes a translation initiation sequence,
IBFP1 encodes a first inclusion body fusion partner comprising an amino acid sequence corresponding to SEQ ID NO: 1, or a variant thereof,
CL1 encodes a first cleavable peptide linker,
ORF encodes a preselected polypeptide,
CL2 encodes a second cleavable peptide linker,
CL3 encodes a third cleavable peptide linker,
IBFP2 encodes a second inclusion body fusion partner comprising an amino acid sequence corresponding to SEQ ID NO: 1, or a variant thereof,
SSC is a suppressable stop codon,
CL4 encodes a fourth cleavable peptide linker,
Ft encodes a fusion tag, and
Tr is a transcription terminator sequence,
wherein each of D or X is independently 0 or an integer of 1 to 4,
wherein R is 0 or an integer of 1 to 2,
wherein each of E, G, L, M, Q, T or W is independently 0 or an integer of 1 to 20,
wherein either one or both of IFP1 or IFP2 is present, and
wherein expression of the expression cassette produces a tandem polypeptide that forms an inclusion body when expressed in a cell.

2. The expression cassette of claim 1 further comprising a nucleic acid sequence that encodes a signal sequence that is operatively coupled at or proximal to the amino-terminus or the carboxyl-terminus of the tandem polypeptide.

3. The expression cassette of claim 2, wherein the signal sequence directs the operably associated tandem polypeptide to a periplasmic space, to an inner membrane, or to an outer membrane of the cell.

4. The expression cassette of claim 2, wherein the signal sequence is obtained from a protein selected from the group consisting of phage fd major coat protein, phage fd minor coat protein, alkaline phosphatase, maltose binding protein, leucine-specific binding protein, β-lactamase, lipoprotein, LamB and OmpA.

5. The expression cassette of claim 1, wherein the nucleic acid sequence of either or both of the IBFP1 or the IBFP2 encodes an inclusion body fusion partner that modulates isolation enhancement of an inclusion body formed from the tandem polypeptide.

6. The expression cassette of claim 6, wherein the isolation enhancement of the inclusion body is self-adhesion, solubility, purification stability, resistance to proteolysis, or altered isoelectric point.

7. The expression cassette of claim 1, wherein the promoter includes an operator selected from the group consisting of a lac operator, a lambda phage operator, a β-galactosidase operator, an arabinose operator, a lexA operator, and a trp operator.

8. The expression cassette of claim 1, wherein the promoter is a T7lac promoter, a tac promoter, a lac promoter, a lambda phage promoter, a heat shock promoter, or a chlorella virus promoter.

9. The expression cassette of claim 1, wherein the translation initiation sequence is obtained from a gene encoding a protein selected from the group consisting of phage T7 gene 10, phage Qβ A, phage Qβ coat, phage Qβ replicase, phage lambda Cro, phage f1 coat, phage φX174 A, phage φX174 B, phage φX174 E, lipoprotein, RecA, GalE, GalT, LacI, LacZ, Ribosomal L10, Ribosomal L7/L12, and RNA polymerase β subunit.

10. The expression cassette of claim 1, wherein each of the first cleavable peptide linker, the second cleavable peptide linker, the third cleavable peptide linker, or the fourth cleavable peptide linker can independently be cleaved by a cleavage agent selected from the group consisting of palladium, cyanogen bromide, clostripain, Thrombin, Trypsin, Trypsin-like protease, Carboxypeptidase, Enterokinase, Kex 2 protease, Omp T protease, Factor Xa protease, Subtilisin, HIV protease, Rhinovirus protease, Furilisin protease, IgA protease, Human Pace protease, Collagenase, Plum pos potyvirus Nia protease, Poliovirus 2Apro protease, Poliovirus 3C protease, Nia protease, Genenase, Furin, Chymotrypsin, Elastase, Subtilisin, Proteinase K, Pepsin, Rennin, microbial aspartic proteases, Papain, Ficin, Bromelain, Collagenase, Thermolysin, Endoprotease Arg-C, Endoprotease Glu-C, Endoprotease Lys-C, Kallikrein and Plasmin.

11. The expression cassette of claim 1, wherein the ORF encodes GLP-1, GLP-2, PTH, GRF, clostripain, or a variant thereof.

12. The expression cassette of claim 1, wherein the ORF contains a suppressible stop codon.

13. The expression cassette of claim 1, wherein the suppressible stop codon is an amber codon or an ochre codon.

14. The expression cassette of claim 13, wherein the suppressible stop codon creates a cleavable peptide linker.

15. The expression cassette of claim 14, wherein the cleavable peptide linker is cleaved by a tissue specific protease.

16. The expression cassette of claim 15, wherein the tissue specific protease is prostate specific antigen.

17. The expression cassette of claim 1, wherein the fusion tag is β-gal, GST, CAT, TrpE, SPA, SPG, MBP, SBD, CBD_{CenA}, CBD_{Cex}, Biotin-binding domain, recA, Flag, poly(Arg), Poly(Asp), Glutamine, poly(His), poly(Phe), poly(Cys), green fluorescent protein, red fluorescent protein, yellow fluorescent protein, cayenne fluorescent protein, biotin, avidin, streptavidin, or an antibody epitope.

18. The expression cassette of claim 1, wherein the termination sequence is a T7 terminator.

19. An RNA produced by transcription of the expression cassette of claim 1.

20. A tandem polypeptide produced by translation of the RNA of claim 19.

21. A nucleic acid construct comprising a vector and the expression cassette of claim 1.

22. The nucleic acid construct of claim 21, wherein the vector is a virus, a plasmid, a phagemid, a bacterial artificial chromosome, a yeast artificial chromosome, a bacteriophage, an f-factor, or a cosmid.

23. A cell comprising the nucleic acid construct of claim 21.

24. The cell of claim 23, wherein the cell is a prokaryotic cell or a eukaryotic cell.

25. The cell of claim 23, wherein the cell is a bacterium.

26. The cell of claim 25, wherein the bacterium is Escherichia coli.

27. The cell of claim 23, wherein the cell is a yeast cell, an insect cell or a mammalian cell.

28. A tandem polypeptide comprising:
a) a first region comprising an inclusion body fusion partner having an amino acid sequence corresponding to SEQ ID NO: 1, or a variant thereof; and
b) a second region not naturally associated with the first region comprising a preselected amino acid sequence.

29. A tandem polypeptide according to claim 28, wherein the first region is at or proximal to the N-terminus of the second region.

30. A tandem polypeptide according to claim 28, wherein the first region is at or proximal to the C-terminus of the second region.

31. A tandem polypeptide according to claim 28, wherein the preselected amino acid sequence corresponds to that of GLP-1, GLP-2, PTH, GRF, clostripain, or a variant thereof.

32. A tandem polypeptide according to claim 28, further comprising a cleavable peptide linker between the first region and the second region.

33. The tandem polypeptide of claim 32, wherein the cleavable peptide linker can be cleaved by a cleavage agent selected from the group consisting of palladium, cyanogen bromide, clostripain, Thrombin, Trypsin, Trypsin-like protease, Carboxypeptidase, Enterokinase, Kex 2 protease, Omp T protease, Factor Xa protease, Subtilisin, HIV protease, Rhinovirus protease, Furilisin protease, IgA protease, Human Pace protease, Collagenase, Plum pos potyvirus Nia protease, Poliovirus 2Apro protease, Poliovirus 3C protease, Nia protease, Genenase, Furin, Chymotrypsin, Elastase, Subtilisin, Proteinase K, Pepsin, Rennin, microbial aspartic proteases, Papain, Ficin, Bromelain, Collagenase, Thermolysin, Endoprotease Arg-C, Endoprotease Glu-C, Endoprotease Lys-C, Kallikrein and Plasmin.

34. The tandem polypeptide according to claim 32, wherein the cleavable peptide linker is cleaved by a tissue specific protease.

35. The tandem polypeptide according to claim 34, wherein the tissue specific protease is prostate specific antigen.

36. The tandem polypeptide according to claim 28, further comprising an operably linked fusion tag.

37. The tandem polypeptide according to claim 36, wherein the fusion tag is a ligand for a cellular receptor.

38. The tandem polypeptide according to claim 37, wherein the fusion tag is insulin.

39. The tandem polypeptide according to claim 36, wherein the fusion tag is β-gal, GST, CAT, TrpE, SPA, SPG, MBP, SBD, CBD_{CenA}, CBD_{Cex}, Biotin-binding domain, recA, Flag, poly(Arg), Poly(Asp), Glutamine, poly(His), poly(Phe), poly(Cys), green fluorescent protein, red fluorescent protein, yellow fluorescent protein, cayenne fluorescent protein, biotin, avidin, streptavidin, or an antibody epitope.

40. A tandem polypeptide according to claim 28, wherein the preselected amino acid sequence is from 2 to 1000 amino acids in length.

41. A tandem polypeptide according to claim 28, wherein the preselected amino acid sequence is from 2 to 100 amino acids in length.

42. A tandem polypeptide according to claim 28, wherein the preselected amino acid sequence is from 2 to 10 amino acids in length.

43. A tandem polypeptide according to claim 28, wherein at least one amino acid in the preselected amino acid sequence is replaced with an amino acid analog.

44. The tandem polypeptide according to claim 28, wherein at least one amino acid in the inclusion body fusion partner is replaced with another amino acid that is a conservative amino acid.

45. The tandem polypeptide of claim 28, wherein the amino acid sequence of the inclusion body fusion partner comprising SEQ ID NO: 1 is altered by replacing at least one amino acid with an acidic amino acid.

46. The tandem polypeptide of claim 28, wherein the amino acid sequence of the inclusion body fusion partner comprising SEQ ID NO: 1 is altered by replacing at least one amino acid with a basic amino acid.

47. The tandem polypeptide of claim 28, wherein the amino acid sequence of the inclusion body fusion partner comprising SEQ ID NO: 1 is altered by replacing at least one amino acid with an aliphatic amino acid.

48. The tandem polypeptide of claim 28, wherein the amino acid sequence of the inclusion body fusion partner comprising SEQ ID NO: 1 is altered by replacing at least one amino acid having a pK value between 4 and 12.

49. The tandem polypeptide of claim 28, wherein the amino acid sequence of the inclusion body fusion partner comprising SEQ ID NO: 1 is altered by replacing at least one amino acid having a pK value between 4 and 7.

50. The tandem polypeptide of claim 28, wherein the amino acid sequence of the inclusion body fusion partner comprising SEQ ID NO: 1 is altered by replacing at least one amino acid having a pK value between 7 and 12.

51. The tandem polypeptide of claim 28, wherein the amino acid sequence of the inclusion body fusion partner comprising SEQ ID NO: 1 is altered by replacing at least one amino acid having a pK value between 6 and 7.

52. The tandem polypeptide of claim 36, further comprising a cleavable peptide linker between the preselected amino acid sequence and the fusion tag.

53. The tandem polypeptide of claim 52, wherein the cleavable peptide linker can be cleaved by a cleavage agent selected from the group consisting of palladium, cyanogen bromide, clostripain, Thrombin, Trypsin, Trypsin-like protease, Carboxypeptidase, Enterokinase, Kex 2 protease, Omp T protease, Factor Xa protease, Subtilisin, HIV protease, Rhinovirus protease, Furilisin protease, IgA protease, Human Pace protease, Collagenase, Plum pos potyvirus Nia protease, Poliovirus 2Apro protease, Poliovirus 3C protease, Nia protease, Genenase, Furin, Chymotrypsin, Elastase, Subtilisin, Proteinase K, Pepsin, Rennin, microbial aspartic proteases, Papain, Ficin, Bromelain, Collagenase, Thermolysin, Endoprotease Arg-C, Endoprotease Glu-C, Endoprotease Lys-C, Kallikrein and Plasmin.

54. A tandem polypeptide of claim 28, wherein the first region causes the tandem polypeptide to form an inclusion body when expressed in a cell.

55. The tandem polypeptide of claim 54, wherein the cell is a bacterium.

56. The tandem polypeptide of claim 55, wherein the bacterium is Escherichia coli.

57. The tandem polypeptide of claim 54, wherein the cell is an insect cell, a yeast cell, or a mammalian cell.

58. A DNA sequence that encodes the tandem polypeptide of claim 28.

59. A method to select an amino acid sequence of an inclusion body fusion partner that confers isolation enhancement to an inclusion body comprising:
a) altering the amino acid sequence of an inclusion body fusion partner comprising SEQ ID NO: 1 that is operably linked to an amino acid sequence not naturally associated with the fusion partner to form a tandem polypeptide that forms the inclusion body, and
b) determining if the inclusion body exhibits enhanced self-adhesion, controllable solubility, purification stability, or resistance to proteolysis.

60. The method of claim 59, wherein the amino acid sequence of the inclusion body fusion partner comprising SEQ ID NO: 1 is altered by replacing at least one amino acid with a conservative amino acid.

61. The method of claim 59, wherein the amino acid sequence of the inclusion body fusion partner comprising SEQ ID NO: 1 is altered by replacing at least one amino acid with a hydrophobic amino acid.

62. The method of claim 59, wherein the amino acid sequence of the inclusion body fusion partner comprising SEQ ID NO: 1 is altered by replacing at least one amino acid with a hydrophilic amino acid.

63. The method of claim 59, wherein the amino acid sequence of the inclusion body fusion partner comprising SEQ ID NO: 1 is altered by replacing at least one amino acid with an uncharged amino acid.

64. The method of claim 59, wherein the amino acid sequence of the inclusion body fusion partner comprising SEQ ID NO: 1 is altered by replacing at least one amino acid having a pK value between 4 and 12.

65. The method of claim 59, wherein the amino acid sequence of the inclusion body fusion partner comprising SEQ ID NO: 1 is altered by replacing at least one amino acid having a pK value between 4 and 7.

66. The method of claim 59, wherein the amino acid sequence of the inclusion body fusion partner comprising SEQ ID NO: 1 is altered by replacing at least one amino acid having a pK value between 7 and 12.

67. The method of claim 59, wherein the amino acid sequence of the inclusion body fusion partner comprising SEQ ID NO: 1 is altered by replacing at least one amino acid having a pK value between 6 and 7.

68. An isolated inclusion body fusion partner having an amino acid sequence corresponding to SEQ ID. NO: 1, and variants thereof.

69. A method to produce a tandem polypeptide comprising:
a) expressing a tandem polypeptide comprising an inclusion body fusion partner that is operably linked to a preselected polypeptide in a cell, and
b) isolating the tandem polypeptide.

70. A nucleic acid sequence that encodes an amino acid sequence comprising SEQ ID NOs: 1 and 2.

71. An amino acid sequence comprising SEQ ID NOs: 1 and 2.

72. A nucleic acid sequence corresponding to SEQ ID NO: 1.

73. A nucleic acid sequence having at least 98% sequence identity to a nucleic acid sequence that encodes an amino acid sequence corresponding to SEQ ID NO: 1.

74. A nucleic acid sequence having at least 90% sequence identity to a nucleic acid sequence that encodes an amino acid sequence corresponding to SEQ ID NO: 1.

75. A nucleic acid sequence having at least 80% sequence identity to a nucleic acid sequence that encodes an amino acid sequence corresponding to SEQ ID NO: 1.

76. A nucleic acid sequence having at least 70% sequence identity to a nucleic acid sequence that encodes an amino acid sequence corresponding to SEQ ID NO: 1.

77. An amino acid sequence corresponding to SEQ ID NO: 1.

78. An amino acid sequence corresponding to SEQ ID NO: 1.

79. An amino acid sequence having at least 98% sequence identity to SEQ ID NO: 1.

80. An amino acid sequence having at least 90% sequence identity to SEQ ID NO: 1.

81. An amino acid sequence having at least 80% sequence identity to SEQ ID NO: 1.

82. An amino acid sequence having at least 70% sequence identity to SEQ ID NO: 1.

83. A tandem polypeptide comprising an inclusion body fusion partner having an amino acid sequence corresponding to SEQ ID NO: 1 operably linked to a preselected polypeptide.
